(19) United States
(12) Patent Application Publication
(10) Pub. No.: US 2024/0218342 A1
(43) Pub. Date: Jul. 4, 2024

(54) SITE-SPECIFIC GLYCAN REMODELING OF LYSOSOMAL ENZYMES AND APPLICATIONS THEREOF

(71) Applicants: Lai-Xi WANG, Ellicott City, MD (US); Xiao ZHANG, College Park, MD (US)

(72) Inventors: Lai-Xi WANG, Ellicott City, MD (US); Xiao ZHANG, College Park, MD (US)

(21) Appl. No.: 18/556,642

(22) PCT Filed: Apr. 25, 2022
(86) PCT No.: PCT/US2022/026225

§ 371 Date: Oct. 20, 2023

Related U.S. Application Data
(60) Provisional application No. 63/178,731, filed on Apr. 23, 2021.
(60) Provisional application No. 63/264,011, filed on Nov. 12, 2021.

(51) Int. Cl. C12N 9/40; A61K 38/00; C07H 17/00; C12N 9/24

(52) U.S. Cl. CPC C12N 9/2465; C07H 17/00; C12N 9/2402; C12Y 302/01022; C12Y 302/01096; A61K 38/00

(57) ABSTRACT

The present disclosure provides compounds useful for enzymatic glycan remodeling of a glycoprotein. Also provided is a method for remodeling a glycoprotein using M6P-glycan oxazolines in a one-pot deglycosylation/transglycosylation process, which may enable site selective M6P-glycan remodeling of glycoproteins to obtain homogeneous products. The remodeled glycoprotein (such as a recombinant human acid α-glucosidase) may have enhanced affinity for the CI-MPR, increased uptake by a cell, and improved therapeutic efficacy compared to the original glycoprotein. A method of treating Pompe disease using a glycan remodeled lysosomal enzyme is also provided.

### CROSS-REFERENCE TO RELATED APPLICATIONS

This application claims priority to U.S. Provisional Application No. 63/178,731, filed on Apr. 23, 2021 and U.S. Provisional Application No. 63/264,011, filed on Nov. 12, 2021, the contents of which hereby are incorporated herein by reference each in their respective entirety.

### STATEMENT REGARDING FEDERALLY SPONSORED RESEARCH

This invention was made with government support under Grant Number R01GM080374E awarded by the National Institutes of Health (NIH). The government has certain rights in the invention.

### SEQUENCE LISTING

A Sequence Listing accompanies this application and is submitted as an ASCII text file of the sequence listing named “171351_00039_ST25.txt” which is 140,992 bytes in size and was created on Apr. 25, 2022. The sequence listing is electronically submitted via EFS-Web with the application and is incorporated herein by reference in its entirety.

### BACKGROUND

Many diseases are associated with the altered or reduced expression of a protein, enzyme, or metabolite. For example, lysosomal storage diseases (LSDs) are a group of inherited metabolic disorders caused by deficiency of respective hydrolases that are responsible for the degradation of substrates stored in lysosomes.¹ The accumulation of undigested macromolecules leads to cell dysfunction and progressive clinical manifestations.² A variety of therapeutic approaches have been attempted for LSDs,³ with intravenous enzyme replacement therapy (ERT) being the most prevalent.⁴ However, the effectiveness of ERT varies among different LSDs. For example, in the case of Pompe disease that is caused by a deficiency of the lysosomal glycogen-degrading enzyme, the acid α-glucosidase (GAA), a very high dose of the recombinant human acid α-glucosidase (rhGAA, Lumizyme) needs to be administered due to its relatively low cellular uptake and poor drug targeting.⁵ The cation-independent mannose-6-phosphate (M6P) receptor (CI-MPR), which continuously traffics between plasma membrane, late endosomes and Trans-Golgi network (TGN), plays a critical role in cellular uptake and intracellular transport of enzymes to lysosomes by recognizing the M6P-containing N-glycans attached to the enzymes.⁶⁻⁹ Thus, enhancement of the CI-MPR-mediated endocytosis represents a major strategy to improve the overall efficiency of the ERT-based treatments.¹⁰⁻¹² Toward this end, several approaches for increasing M6P modification of lysosomal enzymes have been attempted, including chemical conjugation of synthetic M6P-containing glycans,¹³⁻²² construction of non-mammalian based platforms to improve the M6P content,²³⁻²⁶ gene engineering of the glycosylation pathways,^{27, 28} and a chemoenzymatic remodeling approach to introduce synthetic phosphorylated N-glycans.^{29, 30} There are examples that the resulting modified enzymes show increased binding to CI-MPR and enhanced uptake by cells compared with the unmodified enzymes. Despite these promising studies, however, these approaches can be tedious and difficult to control, resulting in mixtures of different glycoforms with varied stability and biological activities or with side effects.

Thus, there remains an unmet need to identify the essential structures of M6P-containing N-glycans that exhibit high affinity for the CI-MPR and to develop site-selective conjugation of the M6P glycan ligands to be able to deliver a therapeutic agent, for example, therapeutic lysosomal enzymes to achieve structurally well-defined products.

### SUMMARY

The present disclosure relates to phosphorylated N-glycan compounds that can be used for one-pot glycan remodeling of glycoproteins, in particular therapeutic lysosomal enzymes, to provide enhanced receptor binding, cellular uptake, and overall therapeutic efficacy.

In one aspect, the disclosure provides a compound of Formula (I), or a salt thereof,

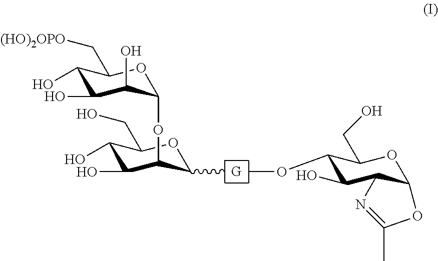

wherein G is sugar moiety or linker.

In another aspect, the disclosure provides a method for remodeling a glycoprotein. The method comprises (a) contacting the glycoprotein with an endoglycosidase selected from the group consisting of wild type Endo A, wild type Endo F3, wild type Endo-CC, and a combination of, thereby producing a deglycosylated intermediate comprising a N-acetylglucosamine (GlcNAc) or core-fucosylated N-acetylglucosamine (Fuca1,6GlcNAc) acceptor from the glycoprotein by a deglycosylation activity of the endoglycosidase to produce a deglycosylated intermediate; and (b) contacting a glycan oxazoline comprising a mannose-6-phosphate (M6P) moiety with the deglycosylated intermediate in the presence of the endoglycosidase, thereby attaching the glycan oxazoline to the N-acetylglucosamine (GlcNAc) or core-fucosylated N-acetylglucosamine (Fuca1,6GlcNAc) acceptor by a transglycosylation activity of the endoglycosidase, thereby producing a remodeled glycoprotein, wherein (a) and (b) are carried out in a one-pot reaction.

In yet another aspect, the disclosure provides a method of enhancing binding affinity of a glycoprotein to a cation-independent M6P receptor (CI-MPR), comprising remodeling the glycoprotein according to the method described herein and contacting the glycoprotein with a cell comprising CI-MPR receptor, thereby enhancing binding affinity of the glycoprotein to the CI-MPR.

In another aspect, the disclosure provides a method of enhancing or increasing uptake of a glycoprotein in a cell. The method comprises (a) remodeling the glycoprotein according to the method described herein, and (b) contacting the cell with the remodeled glycoprotein, thereby enhancing uptake of the glycoprotein in the cell.

In a further aspect, the disclosure provides a glycan-remodeled glycoprotein produced by the method described herein. In some aspect, the glycan-remodeled glycoprotein is a glycan-remodeled lysosomal enzyme.

In yet a further aspect, the disclosure provides a method of treating Pompe disease in a subject in need thereof. The method comprises administering to the subject a pharmaceutically effective amount of the glycan-remodeled lysosomal enzyme described herein.

The foregoing and other aspects and advantages of the embodiments of the present disclosure will appear in from the following description. In the description, reference is made to the accompanying drawings which form a part hereof, and in which there is shown by way of illustration preferred embodiments of the disclosure. Such embodiments are illustrative only, are not intended to be limited, and do not necessarily represent the full scope of the present disclosure, however, and reference is made therefore to the claims herein for interpreting the scope of the teachings of the present disclosure. As such, features of the presently disclosed subject matter will be apparent from the following detailed description and the appended claims that follow.

### List of Abbreviations

The following list of abbreviations are used in the understanding of the present disclosure: lysosomal storage disease (LSD), enzyme replacement therapy (ERT), acid α-glucosidase (GAA), recombinant human acid α-glucosidase (rhGAA), mannose (Man), mannose-6-phosphate (M6P), cation-independent mannose-6-phosphate receptor (CI-MPR), Trans-Golgi network (TGN), High Performance Liquid Chromatography (HPLC), Electrospray Ionization Mass Spectrometry (ESI-MS).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Structures of synthetic M6P-containing N-glycan oxazolines used for structure-activity relationship studies of enzymatic transglycosylation and M6P receptor binding.

FIGS. 2A-2F Glycan analysis (negative mode) and representative sensorgrams of three independent SPR binding experiments of native rhGAA and M6P glycan-remodeled rhGAA (69 and 70). Average K_{D} values for native rhGAA, 69 and 70 were 14.0±3.7 nM, 2.3±0.2 nM and 0.63±0.07 nM, respectively.

FIG. 3 Comparison of the enzyme activity of commercial rhGAA and the M6P glycan-remodeled rhGAA (69 and 70).

FIGS. 4A-4D show the effect of the therapeutic enzymes in GAA KO (KO) myotubes. FIG. 4A shows that GAA activity was measured in cell lysates from untreated-(KO) and treated KO myotubes following incubation with Lumizyme (KO-Lumizyme), Endo-F3 remodeled rhGAA (KO-Endo-F3), and Endo-A remodeled rhGAA (KO-Endo-A). GAA activity in cell lysates of wild type (WT) myotubes was measured for comparison. The KO cells were treated for 24 hours with 5 μM of each of the recombinant enzymes; n=5 for each condition. Statistical significance was determined by one-way ANOVA; the graph represents mean+SD. * p<0.05; **** p<0.0001; ns—statistically insignificant. FIG. 4B shows Western blot analysis (with anti-human GAA antibodies) of muscle cell lysates treated with the recombinant enzymes; GAPDH was used as a loading control. FIG. 4C shows Western blot analysis of the recombinant enzymes with anti-human GAA antibodies. FIG. 4D shows Western blot analysis of WT, untreated- and treated muscle cell lysates with lysosomal (anti-LAMP1) and endosomal (anti-Rab5) antibodies. A decrease in the levels of both markers is observed in Endo-F3- and Endo-A treated cells.

FIGS. 5A-5B show the effect of the therapeutic enzymes on lysosomal swelling and glycogen content in GAA KO (KO) myotubes. FIG. 5A shows confocal images of WT, untreated-(KO) and treated KO myotubes following incubation with Lumizyme (KO-Lumizyme), Endo F3 remodeled rhGAA (KO-Endo-F3), and Endo A remodeled rhGAA (KO-Endo-A). Enlarged LAMP1-positive lysosomes (red) can be detected in untreated-(KO) and in Lumizyme-treated cells but not in WT or KO myotubes treated with the remodeled enzymes. Nuclei are stained with DAPI (blue). Bar: 10 μm. FIG. 5B shows a graph illustrates glycogen levels measured in cell lysates from WT, untreated- and treated KO myotubes. Endo F3 remodeled enzymes restored normal levels of glycogen in KO myotubes; n=5 for each condition. Statistical significance was determined by one-way ANOVA; the graph represents mean±SD. ** p<0.01; **** p<0.0001; ns—statistically insignificant.

FIG. 6. ESI-MS profiles of Glycopeptides 59-66

FIG. 7. ESI-MS profile of Glycoprotein 68

FIG. 8 Representative SPR binding sensorgrams of CI-MPR with M6P-containing glycopeptides. K_{D} values obtained from three independent experiments for compounds 62, 65 and 66 were 70.1±2.1 nM, 82.4±14.1 nM and 53.7±5.6 nM, respectively.

FIG. 9 Representative SPR binding sensorgrams of CI-MPR with remodeled RNase B. K_{D} value obtained from three independent experiments was 15.8±1.9 nM.

FIGS. 10A-10D show the results of glycan analysis of different stages of rhGAA: native rhGAA (FIG. 10A), rhGAA treated with Endo-A (FIG. 10B), Endo-A remodeled rhGAA (FIG. 10C), and Endo-F3 remodeled rhGAA (FIG. 10D). the structures marked with dotted boxes were the residual glycans that not completely removed by buffer exchange

FIG. 11. Transglycosylation of α1,6FucGlcNAc-CD52 with wild-type Endo F3. The reaction was complete within 30 min, and no hydrolysis of the product was observed after 5 h, indicating that Endo F3 would not cleave the newly formed glycosidic bond.

FIG. 12. Representative SPR binding sensorgrams of different stages of rhGAA. Average K_{D} values for native rhGAA, Endo-A remodeled rhGAA (69) and Endo-F3 remodeled rhGAA (70) were 14.0±3.7 nM, 2.3±0.2 nM and 0.63±0.07 nM, respectively.

FIG. 13. MALDI-TOF MS analysis of different glycoforms of rhGAA

FIG. 14. Effect of the therapeutic enzymes on lysosomal swelling in GAA-deficient (KO) myotubes. Confocal images of WT, untreated-(KO) and treated KO myotubes exposed to Lumizyme (KO-Lumizyme), Endo-F3 remodeled rhGAA (KO-Endo-F3), and Endo-A remodeled rhGAA (KO-Endo-A). Both untreated and Lumizyme-treated KO cells contain enlarged LAMP1-positive lysosomes (red); the structures of this size are not seen in WT and KO myotubes treated with the remodeled enzymes (KO-Endo-F3 and KO-Endo-A). Nuclei are stained with DAPI (blue). Bar: 10 μm for all images.

FIG. 15 shows ESI-MS spectrum of glycoprotein 68.

FIG. 16 shows an embodiment of the one pot reaction method of the present disclosure.

FIG. 17 shows scheme 6, Stepwise and one-pot strategy to modify RNase B with the minimal tetrasaccharide oxazoline.

FIG. 18 shows scheme 7, M6P glycan remodeling of rhGAA.

### DETAILED DESCRIPTION

In various embodiments, the present disclosure provides phosphorylated di-, tri-, tetra- and pentasaccharide oxazolines and their donor substrate specificity for endoglycosidases for use in glycan remodeling of peptides and proteins and methods of using such remodeled peptides and proteins as therapeutic agents. Specific phosphorylated oligosaccharide oxazolines (e.g., tetrasaccharide oxazolines) disclosed herein are used as an enzyme substrate in glycan remodeling to prepare high-affinity ligands (e.g., glycoproteins) for cation-independent mannose-6-phosphate receptor (CI-MPR) uptake by cells and the resulting glycan remodeled glycoproteins can be used as improved therapeutics. Remarkably, the glycoengineered peptides and proteins carrying the phosphorylated oligosaccharide are resistant to hydrolysis by the endoglycosidases and their mutants, which enables a simple, one-pot glycan remodeling method that combines the deglycosylation and transglycosylation reactions in one reactor without the need to separate the deglycosylated intermediate and the final protein product (for example, final enzyme product).

The methods described herein can selectively convert high-mannose type or complex type N-glycans in a multiply glycosylated protein into high-affinity M6P-oligosaccharide moieties. The method provides a general approach for a simple, one-pot glycan remodeling approach for improving any glycoprotein for enhanced cellular uptake and improved therapeutic efficacy, for example, for replacement lysosomal enzymes used in enzyme replacement therapy (ERT).

### Definitions

Examples of the present disclosure have been described in terms of one or more preferred embodiments, and it should be appreciated that many equivalents, alternatives, variations, and modifications, aside from those expressly stated, are possible and within the scope of the present disclosure. Before the present disclosure is described, it is understood that embodiments provided in the present disclosure is not limited to the particular methodology, protocols, and reagents described, as these may vary. It is also to be understood that the terminology used herein is for the purpose of describing particular embodiments only, and is not intended to limit the scope of the disclosure.

Unless defined otherwise, all technical and scientific terms used herein have the same meanings as commonly understood by one skilled in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The terms“comprise(s)”, “include(s)”, “having”, “has”, “can”, “contain(s)”, and variants thereof, as used herein, are intended to be open-ended transitional phrases, terms, or words that do not preclude the possibility of additional acts or structures. The singular forms “a”, “an”, and “the” include plural references unless the context clearly dictates otherwise. The present disclosure also contemplates other embodiments “comprising”, “consisting of”, and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Sugar” refers to carbohydrate-containing molecules, including, but not limited to, a monosaccharide, disaccharide, trisaccharide, tetrasaccharide, oligosaccharide, or polysaccharide. A sugar molecule may be substituted, for example, by one or more phosphate groups (e.g., (HO)₂OPO—).

A “sugar moiety” as used herein refers to a monovalent or divalent sugar residue derived from a parent sugar molecule.

### Oxazolines Compounds

Functionalization of therapeutic glycoproteins with mannose-6-phosphate (M6P) glycan ligands represents a major strategy for enhancing the cation-independent M6P receptor (CI-MPR)-mediated cellular uptake, thus improving the overall therapeutic efficacy of the glycoproteins including when being administered to a subject. The present disclosure provides synthetic phosphorylated oxazolines, for example, M6P-containing N-glycan oxazolines, that can be used as donor substrates for transglycosylation to a therapeutic moiety (e.g., glycoprotein) to a make an M6P-containing glycoprotein that can use to target cellular uptake (via CI-MPR) in a cell. The synthetic phosphorylated oxazolines described herein can be used in a one-step method to remodel the glycans on the glycoprotein, and thus provides a “one step” method of conjugating glycoprotein therapeutic molecules.

In the present disclosure, the inventors demonstrate a minimal high-affinity M6P-containing N-glycan ligand for efficient and site-selective conjugation to therapeutic glycoproteins. The chemical synthesis of truncated M6P-glycan oxazolines and their use for enzymatic glycan remodeling of recombinant proteins (for example, human acid α-glucosidase (rhGAA), an enzyme used for treatment of Pompe disease). Structure-activity relationship studies identified an M6P tetrasaccharide oxazoline as the minimal substrate for enzymatic transglycosylation yielding high-affinity M6P glycan ligands for the CI-MPR. Taking advantage of the substrate specificity of endoglycosidases Endo-A and Endo-F3, we found that Endo-A and Endo-F3 could efficiently deglycosylate the respective high-mannose and complex type N-glycans in the glycoprotein and site-selectively transfer the synthetic M6P N-glycan to the deglycosylated glycoprotein without product hydrolysis. This discovery enabled a highly efficient one-pot deglycosylation/transglycosylation strategy for site-selective M6P-glycan remodeling of glycoproteins to obtain a more homogeneous product. The Endo-A and Endo-F3 remodeled glycoproteins maintained full enzyme activity and demonstrated 6- and 20-fold enhanced binding affinities for CI-MPR receptor, respectively.

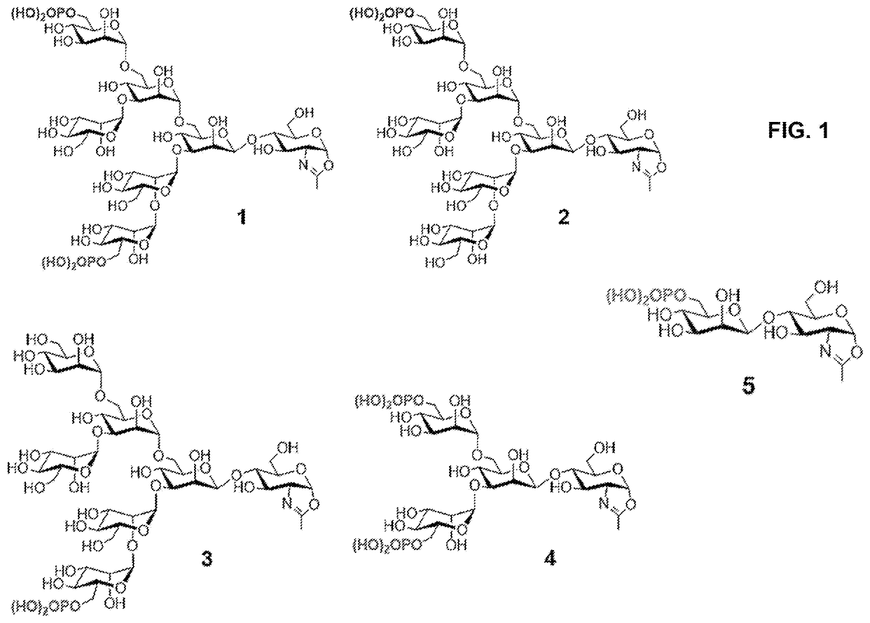

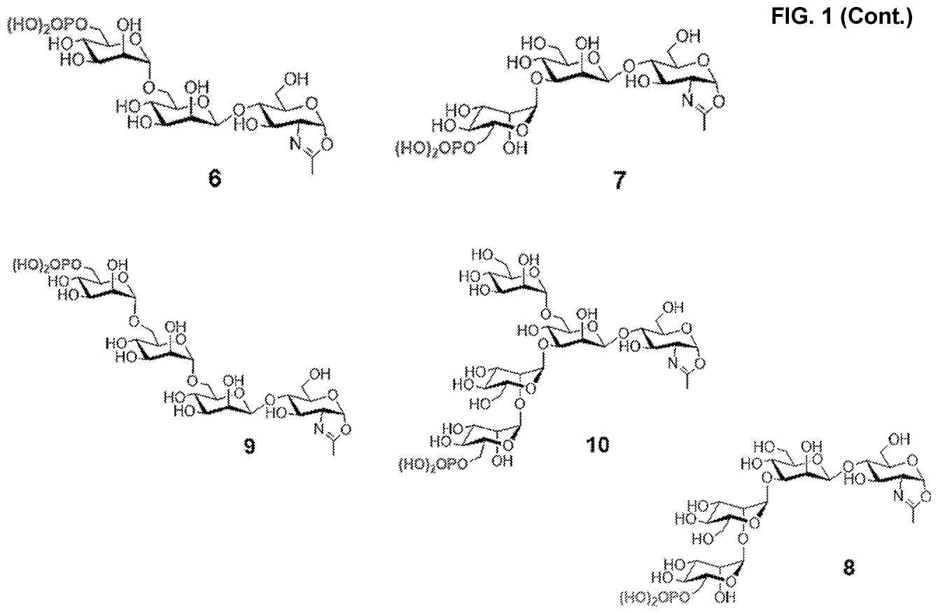

The synthesis of several M6P-containing N-glycan oxazolines (compounds 1-4, FIG. 1) was previously reported and the synthesized compounds were used as donor substrates for endoglycosidase-catalyzed chemoenzymatic synthesis of M6P-containing glycoproteins, using ribonuclease B as a model glycoprotein.³⁰ Binding studies have revealed that a single M6P moiety located at the low α-1,3-branch of the N-glycan context, derived from glycan oxazoline 3, is sufficient for a high-affinity binding to CI-MPR, while the presence of an M6P moiety at the α-1,6-branch, as shown in glycan oxazolines 1 and 2, appears dispensable.³⁰ Despite these findings, the substrate specificity of the M6P-glycan oxazolines in enzymatic transglycosylation and the detailed structure-activity relationship of the M6P glycan ligands for receptor binding remained to be elucidated. In addition, it was not clear what constitutes the minimal M6P-glycan structures for high-affinity CI-MPR binding feasible for a facile enzymatic glycan remodeling of therapeutic lysosomal enzymes. To address these problems, the present disclosure provides the chemical synthesis and evaluation of a series of truncated M6P-containing N-glycan oxazolines, including for example compounds 5-10 (FIG. 1), which may be used as donor substrates for enzymatic transglycosylation and glycan remodeling of glycoproteins, such as therapeutic lysosomal enzymes (e.g., commercial rhGAA, Lumizyme).³¹

In one aspect, the present disclosure provides a compound of Formula (I), or a salt thereof,

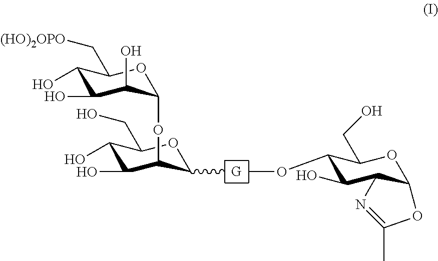

wherein G is a sugar moiety or a linker.

In some embodiments, G is a linker, such as a single bond, a sugar moiety, PEG linker, or a triazole linker. For example, the linker may be a triazole linker formed by azide-alkyne cycloaddition (“click chemistry”). In some embodiments, the linker is not a sugar moiety (or a non-sugar linker). The sugar moiety may, for example, be a linear sugar moiety or a branched sugar moiety. In some preferred embodiments, the sugar moiety is a linear moiety. In general, G may be any structure that does not interfere with or block the activity of an endoglycosidase as disclosed herein. For example, the mannose-6-phosphate-α-1,2-mannose (Man6Pα1,2Man) moiety may provide a binding motif for the endoglycosidase, G may have a structure that does not block or hinder the binding of a compound of Formula (I) to the endoglycosidase, and the endoglycosidase may effectively use the compound of Formula (I) as a substrate.

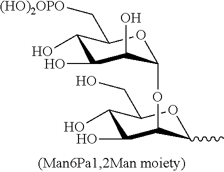

In some embodiments, G is a sugar moiety, such as a monosaccharide moiety, a disaccharide moiety, a trisaccharide moiety, or a tetrasaccharide moiety. In some embodiments, G is a linear sugar moiety. Suitable linear sugar moieties include, for example, a monosaccharide, linear disaccharide, linear trisaccharide or linear tetrasaccharide moiety. In some embodiments, G is not a branched sugar moiety (e.g., compounds of Formula I do not include compound 1 or 3 found in FIG. 1). In some embodiments, G is a monosaccharide moiety, such as a mannose moiety or a glucose moiety. The monosaccharide moiety may be attached to the remainder of the parent molecule through a 1,2-divalent linkage, a 1,3-divalent linkage, a 1,4-divalent linkage, a 1,6-divalent linkage, a 2,3-divalent linkage, a 2,4-divalent linkage, a 2,6-divalent linkage, a 3,4-divalent linkage, a 3,6-divalent linkage, or a 4,6-divalent linkage. In some embodiments, the monosaccharide moiety is a 1,3-mannose moiety or a 1,3-glucose moiety (shown below). The bond at C1 of monosaccharide moiety may be a 1α or 1β configuration.

Linkers include other natural glycosidic bonds, for example, but not limited to, PEG linker, triazole click linker, etc. An alternative approach to that described herein includes the transfer of an azide-tagged sugar, then click a high-affinity M6P-glycan ligand.

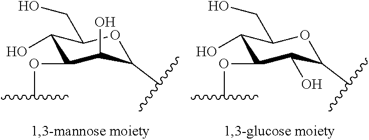

In some embodiments, the compound of Formula (I) has a structure of Formula (I-a), or a salt thereof

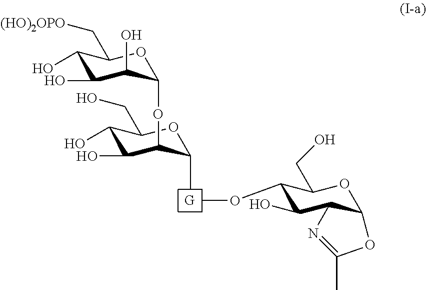

wherein G is as defined in Formula (I).

Suitable compound of Formula (I) may be selected from the group consisting of

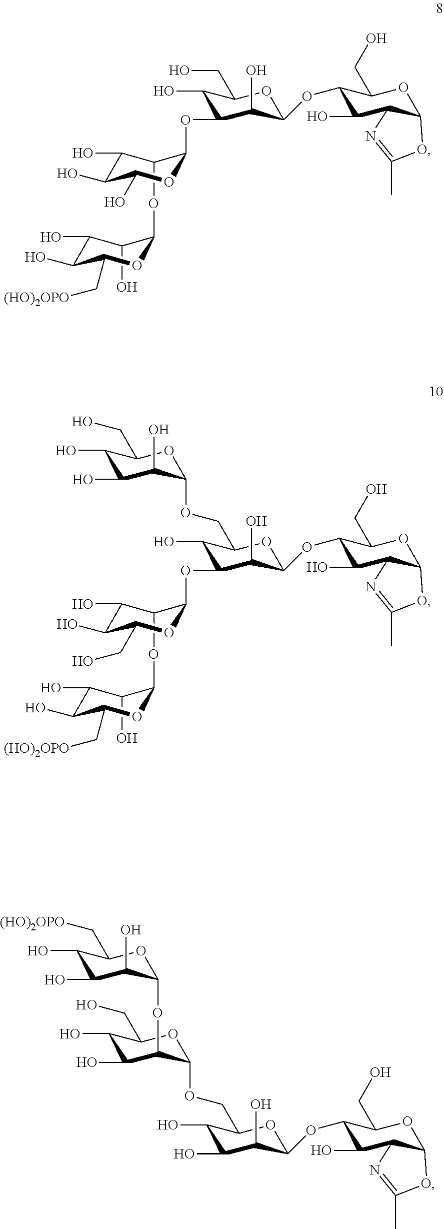

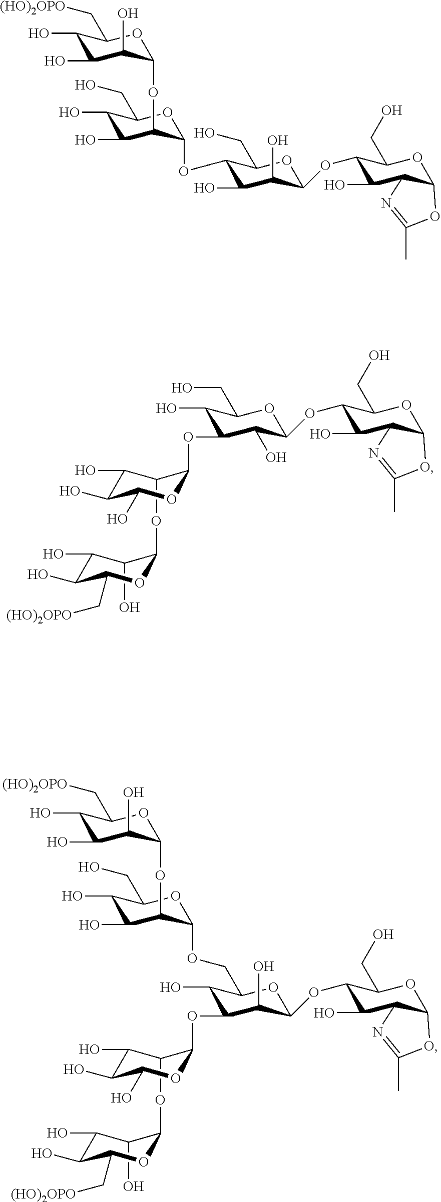

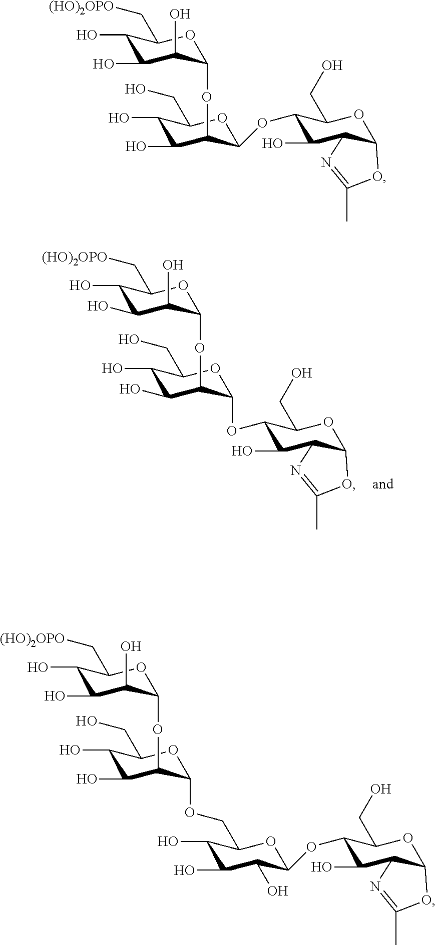

or a salt thereof.

In some embodiments, the compound is

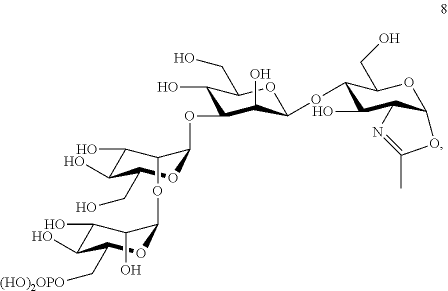

or a salt thereof.

The salt may be, for example, a pharmaceutically acceptable salt. The term “pharmaceutically acceptable salt” refers to salts derived from a variety of organic and inorganic counter ions known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, and phosphoric acid. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, and salicylic acid. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, and aluminum. Organic bases from which salts can be derived include, for example, primary, secondary, and tertiary amines, substituted amines including naturally occurring substituted amines, cyclic amines, and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The present disclosure further provides remodeled glycoproteins. The remodeled glycoproteins comprise one or more of the compounds (e.g., glycan oxazolines) described herein. The glycoprotein may be any protein or peptide that comprises a modifiable sugar (e.g., mannose or N-glycans) that is able to bind endoglycosidase that is capable of deglycosylation and transglycosylation in the same reaction mixture, and without a purification of an intermediate. In some embodiments, the remodeled glycoprotein is remodeled to include a mannose-6-phosphate group, which is capable of specific binding to its receptor on cell surfaces.

### Method of Remodeling Glycoprotein

The present disclosure also relates to a method for selectively converting high-mannose type or complex type N-glycans in a multiply glycosylated protein into high-affinity M6P-oligosaccharide moieties. The method provides a general approach for a simple, one-pot glycan remodeling approach for glycoproteins, such as therapeutic lysosomal enzymes, thereby enhancing their cellular uptake and overall therapeutic efficacy. These methods provide a one pot process to increase the yield, reduce purification steps and provide a purified product that, in some embodiments, has increased therapeutic efficacy. Further, this procedure allows for the ability to target the therapeutic to the lysosymal compartments in a cell.

In particular, the method may employ high-affinity tetrasaccharide oxazoline and wild type endoglycosidases, such as Endo-A from Arthrobacter protophormia or Endo F3 from Elizabethkingia meningoseptica to perform transglycosylation on multiply glycosylated proteins without hydrolysis of the resulting products. In specific embodiments, the remarkable difference in hydrolytic activity of the wild-type Endo-A/Endo-F3 toward the parent enzyme and the resulting transglycosylation product enables a simple, one-pot process that combines the protein deglycosylation and transglycosylation without the need to separate the deglycosylation intermediate and the enzyme. This method can selectively convert high-mannose type or complex type N-glycans in a multiply glycosylated protein into high-affinity M6P-oligosaccharide moieties, thus can be used to enhance cellular uptake and overall therapeutic efficacy of any glycoprotein, for example, lysosomal enzymes used in enzyme replacement therapy (ERT).

In one aspect, the present disclosure provides a method for remodeling a glycoprotein, comprising: a) contacting the glycoprotein with an endoglycosidase thereby producing a deglycosylated intermediate comprising a N-acetylglucosamine (GlcNAc) or core-fucosylated N-acetylglucosamine (Fuca1,6GlcNAc) acceptor from the glycoprotein by a deglycosylation activity of the endoglycosidase to produce a deglycosylated intermediate; and (b) contacting a glycan oxazoline comprising a mannose-6-phosphate (M6P) moiety with the deglycosylated intermediate in the presence of the endoglycosidase, thereby attaching the glycan oxazoline to the N-acetylglucosamine (GlcNAc) or core-fucosylated N-acetylglucosamine (Fuca1,6GlcNAc) acceptor by a transglycosylation activity of the endoglycosidase, thereby producing a remodeled glycoprotein, wherein (a) and (b) are carried out in a one-pot reaction (e.g., in a single reaction mixture without any purification steps to remove the endoglycosidase). In some embodiments, the endoglycosidase is selected from the group consisting of wild type Endo A, wild type Endo F3, wild type Endo-CC, and a combination of. Further, the resultant remodeled glycoprotein, specifically, enzymatic glycoproteins and especially lysosomal enzymes are capable of being targeted to the lysozyme through the specific and increased binding to the CI-MPR receptor.

The term “one-pot reaction” refers to a reaction which is performed without isolating or separating any intermediate product. In other words, the intermediate product is produced and then used in situ, as the term is understood in the art, for the next step of the reaction. Routine experimental procedures that do not remove the intermediate product from the reaction mixture (e.g., evaporation of solvent, dialysis) may be included in a one-pot reaction. The order in which reagents for a one-pot reaction (e.g., starting materials, enzymes, substrates) are added are not limited. For example, the reagents may be added sequentially as the reaction progresses or the reagents may be added all together at the beginning of the reaction.

The one pot method described herein can be used for any glycoprotein (e.g., protein that contains one or more modifiable sugar moieties). The glycoproteins only requirement is to have one or more modifiable sugar moieties, as demonstrated in the examples and described herein. For example, the one or more sugar moieties may be any N-glycans, including high-mannose type, complex type, hybrid type, or their truncated forms.

The glycoproteins may be proteins or peptides that are used as therapeutics and targeted to the lysosyme. In some embodiments, the glycoproteins are enzymatic proteins. In other embodiments, the glycoproteins may be any protein that is to be targeted to lysozymes (e.g., enzymes).

In some embodiments, the glycoprotein is a lysosomal enzyme. For example, the lysosomal enzyme may be a therapeutic enzyme for enzymatic replacement therapy (ERT). In some embodiments, the present method remodels of the lysosomal enzyme, resulting in site-selective introduction of a high-affinity ligand (e.g., an M6P ligand), which improves the therapeutic efficacy of the therapeutic enzyme.

Suitable lysosomal enzymes include, but are not limited to, α-galactosidase A, acid ceramidase, acid α-L-fucosidase, acid β-glucosidase, acid β-galactosidase, iduronate-2-sulfatase, α-L-iduronidase, galactocerebrosidase, acid α-mannosidase, acid β-mannosidase, arylsulfatase B, arylsulfatase A, N-acetylgalactosamine-6-sulfate sulfatase (N-acetylgalactosamine-6-sulfatase, or galactose-6-sulfatase), acid β-galactosidase, acid sphingomyelinase, acid α-glucosidase (α-glucosidase), β-hexosaminidase B, heparan N-sulfatase, α-N-acetylglucosaminidase, acetyl-CoA: α-glucosaminide N-acetyltransferase, N-acetylglucosaminide-6-sulfate sulfatase, α-N-acetylgalactosaminidase, sialidase, β-glucuronidase, β-hexosaminidase A, and a combination thereof. In some embodiments, the lysosomal enzyme comprises at least one asparagine (N)-linked glycan. In some embodiments, the lysosomal enzyme is an acid α-glucosidase (α-glucosidase).

For example, Lumizyme® (alglucosidase alfa, Genzyme Corporation) is a therapeutic lysosomal enzyme that carries 7 different N-glycans and currently is used for the treatment of Pompe disease. The structure-activity relationship study revealed a Man6P-α1,2-Man disaccharide moiety in the synthetic N-glycans as a structural motif for high-affinity binding to the CI-MPR and identified a tetrasaccharide oxazoline as a donor substrate for enzymatic transglycosylation to provide high-affinity M6P glycan ligands for the CI-MPR. In specific embodiments, the present disclosure provides a method for one-pot fand site-selective glycan remodeling of the multiply glycosylated rhGAA to produce a more homogeneous glycoengineered enzyme that showed up to 20-fold enhanced binding affinities for the CI-MPR over the commercial Lumizyme. The present disclosure also demonstrates significantly enhanced cellular uptake of M6P-glycan remodeled rhGAA in a cell model system for Pompe disease, leading to much more efficient degradation of glycogen in lysosomes than the commercial Lumizyme under the same conditions.

The endoglycosidase as disclosed herein may have a deglycosylation activity, transglycosylation activity, or both. In some embodiments, the endoglycosidase is wild type Endo A. The wild type Endo A may comprise a sequence of SEQ ID NO:1. In some embodiments, the wild type Endo A removes high-mannose and hybrid type glycans from a glycoprotein (e.g., a lysosomal enzyme) without affecting complex-type glycans.

In some embodiments, the endoglycosidase is wild type Endo F3. The wild type Endo F3 may comprise a sequence of SEQ ID NO:2. In some embodiments, the wild type Endo F3 removes core-fucosylated complex-type glycans from the glycoprotein (e.g., lysosomal enzyme) without affecting high-mannose or hybrid type glycans.

In some embodiments, the endoglycosidase is wild type Endo-CC. The wild type Endo-CC may comprise a sequence of SEQ ID NO:3. In some embodiments, the wild type Endo-CC removes high-mannose type and biantennary complex type glycans from the glycoprotein (e.g., lysosomal enzyme) without affecting core-fucosylated complex-type glycans or higher branched complex type glycans.

In some embodiments, the endoglycosidase is a combination of the wild type Endo A and the wild type Endo F3. The ratio of the activity of the wild type Endo A (Unit) to that of the wild type Endo F3 in the combination may be about 0.1:99.9 to about 99.9:0.1, such as about 1:99, about 10:90, about 20:80, about 30:70, about 40:60, about 50:50, about 60:40, about 70:30, about 80:20, about 90:10, or about 99:1.

The glycan oxazoline may be any suitable compound comprising a mannose-6-phosphate (M6P) moiety that can be used as a glycan donner substrate in the transglycosylation reaction mediated by the endoglycosidase. In some embodiments, the glycan oxazoline comprises at least one Man6Pa1,2Man moiety, as disclosed herein.

In some embodiments, the glycan oxazoline has a structure of formula (I), or a salt thereof,

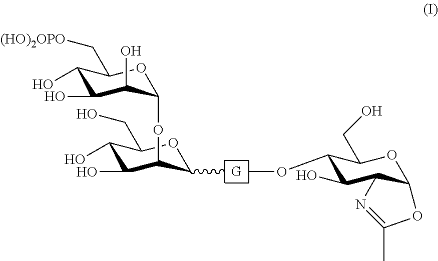

wherein G is sugar moiety or a linker, as disclosed herein.

In some embodiments, G is a sugar moiety, such as a monosaccharide moiety, a disaccharide moiety, a trisaccharide moiety, or a tetrasaccharide moiety. In some embodiments, G is a monosaccharide moiety, such as a mannose moiety or a glucose moiety as disclosed herein. In some embodiments, G is a linker that links the two sugar moieties via a bond.

Suitable glycan oxazoline compounds include, for example, one selected from the group consisting of:

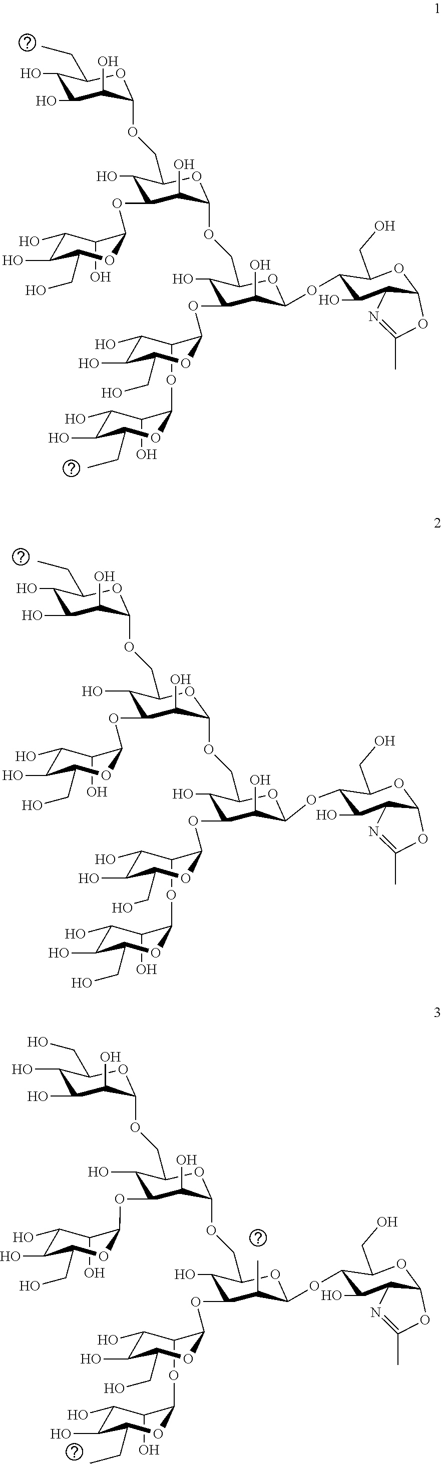

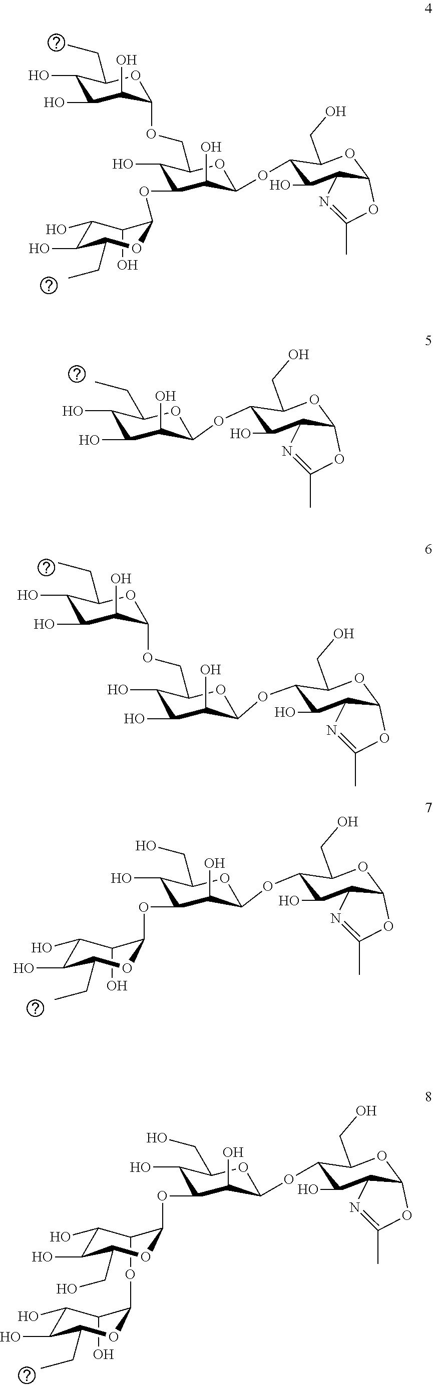

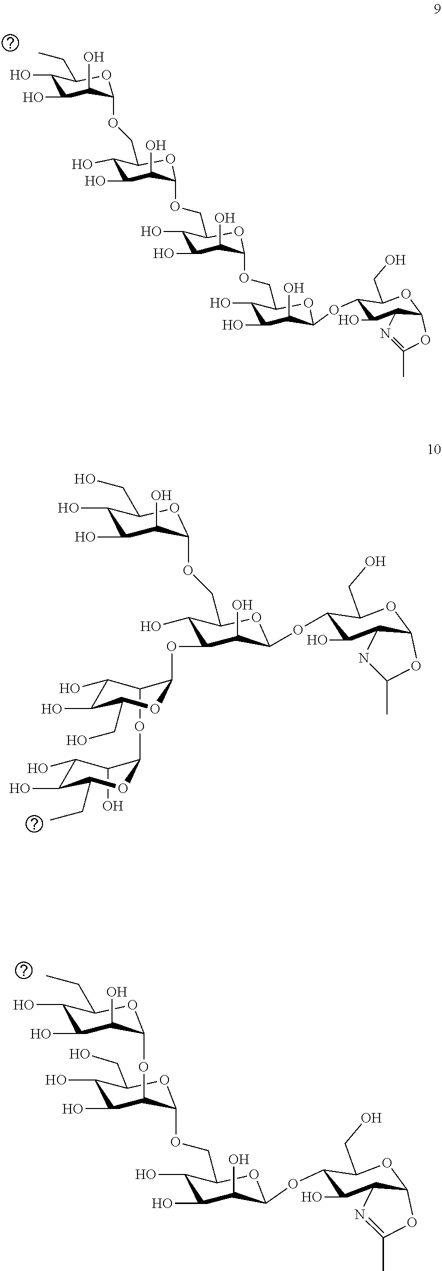

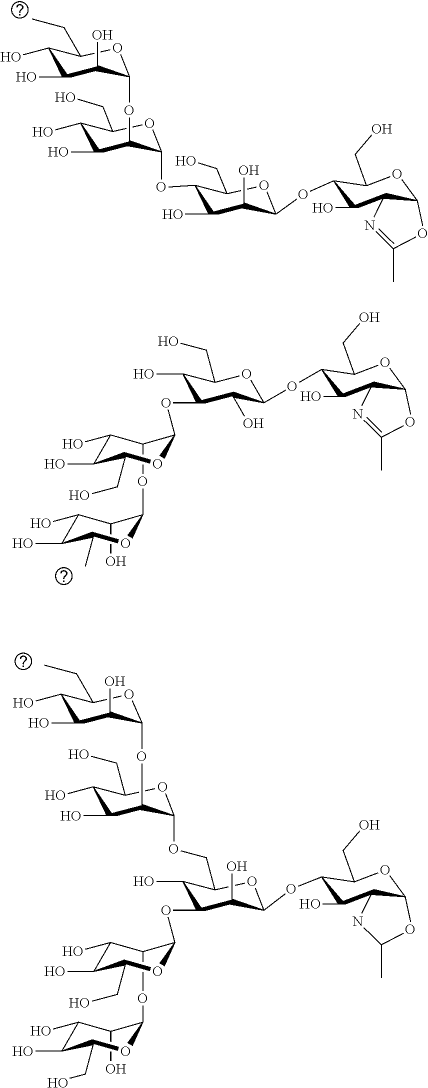

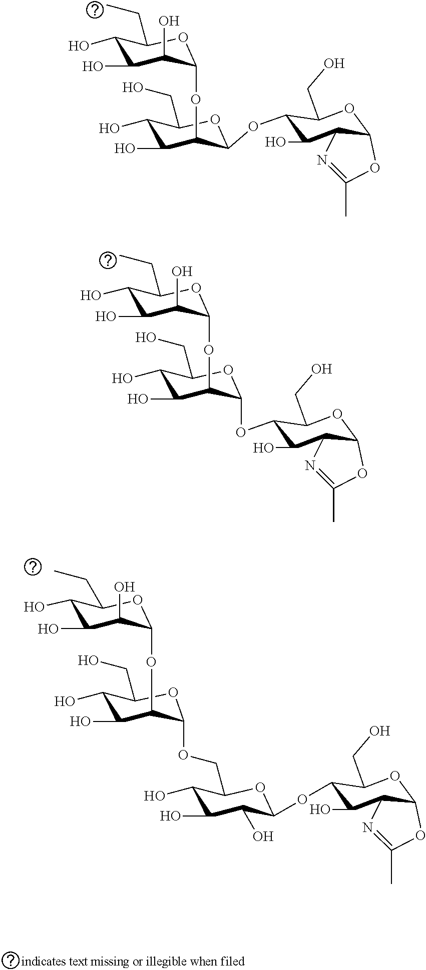

or a salt thereof.

In some embodiments, the glycan oxazoline is

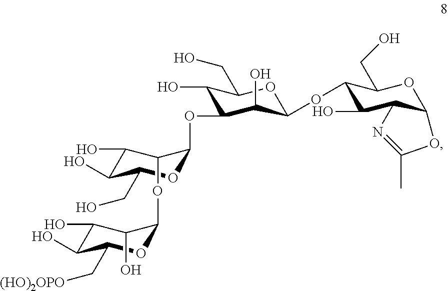

or a salt thereof.

The present disclosure also provides remodeled glycoproteins comprising one or more of the glycan oxazoline linked to a mannose-6 phosphate (M6P) moiety as described herein. In one embodiment, provided is a method of enhancing binding affinity of a glycoprotein to a cation-independent M6P receptor (CI-MPR), comprising remodeling the glycoprotein according to the method described herein; and contacting the remodeled glycoprotein with a cell comprising CI-MPR receptor, thereby enhancing binding affinity of the glycoprotein to the CI-MPR.

The remodeled glycoprotein may have a binding affinity to CI-MPR that is at least 25% higher than that of the original glycoprotein (i.e. without glycan remodeling). For example, the binding affinity of the remodeled glycoprotein may be at least 50%, at least 100%, at least 200%, at least 500%, at least 1000%, at least 5000%, or at least 10000% higher than that of the original glycoprotein.

In another embodiment, provided is a method of enhancing or increasing uptake of a glycoprotein in a cell, comprising remodeling the glycoprotein according to the method described herein, and contacting the cell with the remodeled glycoprotein, thereby enhancing uptake of the glycoprotein in the cell.

The uptake of the remodeled glycoprotein by the cell may be at least 25% higher than that of the original glycoprotein (i.e. without glycan remodeling). For example, the uptake of the remodeled glycoprotein by the cell may be at least 50%, at least 100%, at least 200%, at least 500%, at least 1000%, at least 5000%, or at least 10000% higher than that of the original glycoprotein.

Suitable glycoproteins for these methods may include, but are not limited to lysosomal enzymes, such as acid «-glucosidase (α-glucosidase). For example, a lysosomal enzyme remodeled by the present method may have an increased binding affinity to CI-MPR and/or an uptake by the cell that is at least 100% higher (such as about 500%, about 1000%, about 2000%, or about 5000% higher) than the corresponding value of the original lysosomal enzyme.

The cell may be in vitro or in vivo. The cell may be derived from a tissue or a cell line, such as cultured cells. In some embodiments, the cell is from a mammalian cell line. For example, the cell may be a heart cell, a brain cell, a muscle cell, a liver cell, or an endothelium cell. In some embodiments, the cell is a diseased cell, such as a cell model for Pompe disease, heart disease, or cancer. In some embodiments, the lysosomal enzyme is acid α-glucosidase (α-glucosidase), and the cell is a muscle cell.

### Remodeled Glycoproteins

In another aspect, provided is glycan-remodeled glycoprotein produced by the remodeling method as described herein. The glycan-remodeled glycoproteins include, but are not limited to glycan-remodeled enzymes, including, for example, lysosomal enzymes. In some embodiments, the glycan-remodeled lysosomal enzyme is a glycan-remodeled acid α-glucosidase (α-glucosidase). The glycan-remodeled glycoproteins may be targeted to the lysosomal compartments of cells.

The glycan-remodeled glycoproteins (e.g., the glycan-remodeled lysosomal enzyme) as described herein may be used as a therapeutic enzyme. In one aspect, the present disclosure also provides a pharmaceutical composition comprising a therapeutically effective amounts of a glycan-remodeled glycoproteins (e.g., a glycan-remodeled lysosomal enzyme) as described herein and a pharmaceutically acceptable carrier.

A “therapeutically effective amount” refers to an amount effective, at dosages and for periods of time necessary, to achieve the desired therapeutic result, such as beneficial or desirable biological and/or clinical results. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the present pharmaceutical composition are outweighed by the therapeutically beneficial effects.

For example, a therapeutically effective amount of a glycan-remodeled glycoproteins (e.g., a glycan-remodeled lysosomal enzyme) of the present disclosure, may be about 1 mg/kg to about 1000 mg/kg, such as about 5 mg/kg to about 1000 mg/kg, about 10 mg/kg to about 1000 mg/kg, about 10 mg/kg to about 800 mg/kg, about 10 mg/kg to about 400 mg/kg, about 10 mg/kg to about 200 mg/kg, about 10 mg/kg to about 100 mg/kg, about 20 mg/kg to about 800 mg/kg, about 20 mg/kg to about 400 mg/kg, about 20 mg/kg to about 200 mg/kg, about 30 mg/kg to about 600 mg/kg, or about 30 mg/kg to about 300 mg/kg. The therapeutically effective amount may be, for example, about 1 mg/kg, about 10 mg/kg, about 20 mg/kg, about 50 mg/kg, about 100 mg/kg, about 200 mg/kg, about 500 mg/kg, or about 800 mg/kg. In some embodiments, the therapeutically effective amount is about 20 mg/kg.

The term “pharmaceutically acceptable carrier,” as used herein, means a non-toxic, inert solid, semi-solid or liquid filler, diluent, encapsulating material or formulation auxiliary of any type. Suitable pharmaceutically acceptable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutically acceptable carriers are well known to those skilled in the art and include, but are not limited to, 0.01 to 0.1 M and preferably 0.05M phosphate buffer or 0.9% saline. Additionally, such pharmaceutically acceptable carriers may be aqueous or non-aqueous solutions, suspensions, and emulsions. Examples of nonaqueous solvents are propylene glycol, polyethylene glycol, vegetable oils such as olive oil, and injectable organic esters such as ethyl oleate. Aqueous carriers include isotonic solutions, alcoholic/aqueous solutions, emulsions or suspensions, including saline and buffered media.

Compositions of the present disclosure may include liquids, lyophilized, or otherwise dried formulations and may include diluents of various buffer content (e.g., Tris-HCl, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the polypeptide, complexation with metal ions, or incorporation of the material into or onto particulate preparations of polymeric compounds such as polylactic acid, polyglycolic acid, hydrogels, etc., or onto liposomes, microemulsions, micelles, milamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils).

The compositions can be sterilized by conventional, well-known sterilization techniques. The compositions may contain pharmaceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like.

The disclosed glycan-remodeled glycoproteins (e.g., glycan-remodeled lysosomal enzyme) may be formulated for administration by, for example, solid dosing, eyedrop, in a topical oil-based formulation, injection, inhalation (either through the mouth or the nose), implants, oral, buccal, parenteral, or rectal administration. Techniques and formulations may generally be found in “Remington's Pharmaceutical Sciences”, (Meade Publishing Co., Easton, Pa.). Therapeutic compositions typically are sterile and stable under the conditions of manufacture and storage.

The route by which the present composition is administered and the form of the composition may dictate the type of carrier to be used. The composition may be administered, for example, by oral, rectal, sublingual, parenteral, or topical administration. Parenteral administration may include, for example, intramuscular, intraperitoneal, intravenous, and transdermal administration. The composition may be in a variety of forms, suitable, for example, for systemic administration (e.g., oral, rectal, sublingual, buccal, implants, or parenteral) or topical administration (e.g., dermal, pulmonary, nasal, aural, ocular, liposome delivery systems, or iontophoresis).

Carriers for systemic administration may include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil, and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmellose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active agents and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound, and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not critical for the purposes of this invention.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methylcellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

The disclosed composition can be topically administered. Topical compositions that can be applied locally to the skin may be in any form including solids, solutions, oils, creams, ointments, gels, lotions, shampoos, leave-on and rinse-out hair conditioners, milks, cleansers, moisturizers, sprays, skin patches, and the like. Topical compositions may include: a disclosed glycan-remodeled glycoproteins (e.g., a glycan-remodeled lysosomal enzyme) and a carrier. The carrier of the topical composition preferably aids penetration of the compounds into the skin. The carrier may further include one or more optional components.

The amount of the carrier employed in conjunction with a disclosed glycan-remodeled glycoproteins (e.g., a glycan-remodeled lysosomal enzyme) is sufficient to provide a practical quantity of composition for administration per unit dose of the medicament. Techniques and compositions for making dosage forms useful in the present methods are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2nd Ed., (1976).

A carrier may include a single ingredient or a combination of two or more ingredients. In the topical compositions, the carrier includes a topical carrier. Suitable topical carriers include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, carriers for skin applications include propylene glycol, dimethyl isosorbide, and water, and even more particularly, phosphate buffered saline, isotonic water, deionized water, monofunctional alcohols, and symmetrical alcohols.

The carrier of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%.

The amount of thickener(s) in a topical composition is typically about 0% to about 95%.

Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%.

Suitable pH adjusting additives include HCl or NaOH in amounts sufficient to adjust the pH of a topical pharmaceutical composition.

In some embodiments, the present pharmaceutical composition is suitable for intravenous administration. For example, the composition may be supplied (e.g., in a vial) as a sterile, nonpyrogenic, lyophilized cake or powder for reconstitution with sterile water for injection. The composition may include the glycan-remodeled glycoproteins (e.g., glycan-remodeled lysosomal enzyme) (e.g., 10-50 mg), mannitol (e.g., 100-500 mg), polysorbate 80 (e.g., 0.1-1 mg), sodium phosphate dibasic heptahydrate (e.g., 5-20 mg), sodium phosphate monobasic monohydrate (e.g., 10-50 mg). The composition may be reconstituted into an injectable solution.

### Method of Pharmaceutical Use

The glycan-remodeled glycoproteins (e.g., glycan-remodeled lysosomal enzyme) may be used for treating a disease. The disease may be associated with, or caused by, a deficiency of the glycoprotein, for example, an enzyme deficiency.

Specifically, diseases which are caused by the deficiency in a single protein, e.g., single glycoprotein. For example, most lysosomal storage diseases are caused by a deficiency or dysregulation of a single lysosomal enzyme. Thus, in one embodiment, the present disclosure provides methods of treating a lysosomal storage disease comprising administering a glycan-remodeled lysosomal enzyme as described herein. The glycan-remodeled lysosomal enzyme comprises an M6P binding moiety that is capable of binding to cation-independent mannose 6-phosphate receptor (CI-MPR) on the surface of cells. This allows for the targeting and delivery of the glycan-remodeled lysosomal enzymes to the lysosome of the cell via the CI-MPR.

In some embodiment, the disclosure provides a method of treating a lysosomal storage disease, the method comprising administering an effective amount of a glycan-remodeled lysosomal enzyme comprising an M6P moiety capable of binding to CI-MPR and treating the lysosomal storage disease.

Suitable lysosomal storage diseases are listed in Table 1, along with the enzyme glycoprotein that is able to be remodeled by the methods described herein for use in treating the lysosomal storage disease.

In one example, and as described in the Examples below, the lysosomal storage disease is Pompe disease, and the methods described herein are used to remodel α-glucosidase (rhGAA). rhGAA is used as a model therapeutic enzyme to demonstrate the efficiency of the glycan remodeling method. But this method should be applicable for the glycan remodeling of all the lysosomal enzymes for the site-selective introduction of the high-affinity M6P ligands for improving the therapeutic efficacy of those therapeutic enzymes used in enzymatic replacement therapy (ERT), as shown in Table 2.42

TABLE 2
Examples of LSDs and Corresponding Lysosomal Hydrolases

| Lysosomal Storage | | |
|---|---|---|
| Disorder | Defective Enzyme | Protein Sequence |
| Fabry | α-Galactosidase A | UniProtKB - P06280 (SEQ |
| | | ID NO: 4) |
| Farber | Acid ceramidase | UniProtKB - Q13510 (SEQ |
| | | ID NO: 5) |
| Fucosidosis | Acid α-L-fucosidase | UniProtKB - P04066 (SEQ |
| | | ID NO: 6) |
| Gaucher types 1, 2, and 3 | Acid β-glucosidase | UniProtKB - P04062 (SEQ |
| | | ID NO: 7) |
| GM1 gangliosidosis | Acid β-galactosidase | UniProtKB - P16278 (SEQ |
| | | ID NO: 8) |
| Hunter | Iduronate-2-sulfatase | UniProtKB - P22304 (SEQ |
| (Mucopolysaccharidosis | | ID NO: 9) |
| (MPS) II) | | |
| Hurler-Schele, Hurler, | α-L-Iduronidase | UniProtKB - P35475 (SEQ |
| Schele (MPS I) | | ID NO: 10) |
| Krabbe | Galactocerebrosidase | UniProtKB - P54803 (SEQ |
| | | ID NO: 11) |
| α-Mannosidosis | Acid α-mannosidase | UniProtKB - O00754 (SEQ |
| | | ID NO: 12) |
| β-Mannosidosis | Acid β-mannosidase | UniProtKB - O00462 (SEQ |
| | | ID NO: 13) |
| Maroteaux-Larny (MPS | Arylsulfatase B | UniProtKB - P15289 (SEQ |
| VI) | | ID NO: 14) |
| Metachromatic | Arylsulfatase A | UniProtKB - P15289 (SEQ |
| leukodystrophy | | ID NO: 15) |
| Morquio A (MPS IV) | N-Acetylgalactosamine-6- | UniProtKB - P34059 (SEQ |
| | sulfate sulfatase | ID NO: 16) |
| | (N-acetylgalactosamine-6- | |
| | sulfatase) | |
| | (galactose-6-sulfatase) | |
| Morquio B (MPS IV) | Acid β-galactosidase | UniProtKB - P16278 (SEQ |
| | | ID NO: 8) |
| Niemann-Pick A and B | Acid sphingomyelinase | UniProtKB - P17405 (SEQ |
| | | ID NO: 17) |
| Pompe | Acid α-glucosidase (α- | UniProtKB - P10253 (SEQ |
| | glucosidase) | ID NO: 18) |
| Sandhoff | β-Hexosaminidase B | UniProtKB - P07686 (SEQ |
| | | ID NO: 19) |
| Sanfilippo A (MPS III) | Heparan N-sulfatase | UniProtKB - Q68CP4 (SEQ |
| | | ID NO: 20) |
| Sanfilippo B (MPS III) | α-N-Acetylglucosaminidase | UniProtKB - P54802 (SEQ |
| | | ID NO: 21) |
| Sanfilippo C (MPS III) | Acetyl-CoA: α- | UniProtKB - Q68CP4 (SEQ |
| | glucosaminide N- | ID NO: 22) |
| | acetyltransferase | |
| Sanfilippo D (MPS III) | N-Acetylglucosaminide-6- | UniProtKB - P15586 (SEQ |
| | sulfate sulfatase | ID NO: 23) |
| Schindler-Kanzaki | α-N-acetylgalactosaminidase | UniProtKB - P17050 (SEQ |
| | | ID NO: 24) |
| Sialidosis | Sialidase | UniProtKB - Q99519 (SEQ |
| | | ID NO: 25) |
| Sly (MPS VII) | β-Glucuronidase | UniProtKB - P08236 (SEQ |
| | | ID NO: 26) |
| Tay-Sachs | β-Hexosaminidase A | UniProtKB - P07686 (SEQ |
| | | ID NO: 27) |

Protein sequences are incorporated by reference in their entirety.

For example, the disease may be Pompe disease, which is caused by a deficiency of a lysosomal enzyme, such as acid α-glucosidase (GAA). Pompe disease is an inherited disorder resulting from the buildup of a complex sugar called glycogen in the body's cells resulting in the accumulation of glycogen in certain organs and tissues, especially muscles, which impairs their ability to function normally. Mutations within the GAA gene cause Pompe disease as the GAA gene provides instructions for producing an enzyme called acid α-glucosidase (also known as acid maltase). This enzyme is active in lysosomes which serve as recycling centers within cells. The enzyme normally breaks down glycogen in lysosomes into a simpler sugar called glucose, which is the main energy source for most cells.

In one aspect, the present disclosure provides a method of treating Pompe disease in a subject in need thereof, comprising administering to the subject a pharmaceutically effective amount of the glycan-remodeled lysosomal enzyme as described herein.

The subject may be a mammal, in particular a human.

For purposes of the present invention, “treating” or “treatment” describes the management and care of a subject for combating a disease, condition, or disorder. Treating includes the administration of an active agent (such as a therapeutic enzyme or a pharmaceutical composition as disclosed herein) for preventing the onset of the symptoms or complications, alleviating the symptoms or complications, or eliminating the disease, condition, or disorder. As used herein, and as well understood in the art, “treatment” or “treating” is also an approach for obtaining beneficial or desired results, including clinical results. Beneficial or desired clinical results can include, but are not limited to, alleviation or amelioration of one or more symptoms or conditions, diminishment of the extent of disease, stabilized (i.e., not worsening) state of disease, preventing the spread of disease, delay or slowing of disease progression, amelioration or palliation of the disease state, and remission (whether partial or total), whether detectable or undetectable. “Treatment” can also mean prolonging survival as compared to expected survival if not receiving treatment. Further, any of the treatment methods or uses described herein can be formulated alone or for contemporaneous administration with other agents or therapies.

As used herein, the phrase “effective amount” or “therapeutically effective amount” or a “sufficient amount” of a glycoprotein or composition of the present application is a quantity sufficient to, when administered to the subject, effect beneficial or desired results, including clinical results, and, as such, an “effective amount” or synonym thereto depends upon the context in which it is being applied. The amount given should be varied depending upon various factors, such as the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of the present disclosure is an amount, which results in a beneficial or desired result in a subject as compared to a control. As defined herein, a therapeutically effective amount of a compound of the present disclosure may be readily determined by one skilled in the art by routine methods known in the art. Dosage regime may be adjusted to provide the optimum therapeutic response.

The glycan-remodeled lysosomal enzyme may be administered, for example, in a pharmaceutical composition as described herein. In some embodiments, the glycan-remodeled lysosomal enzyme is administered intravenously. In some embodiments, the glycan-remodeled lysosomal enzyme is administered orally.

In particular embodiments, the glycan-remodeled lysosomal enzyme is a glycan-remodeled acid-glucosidase (α-glucosidase). The glycan-remodeled lysosomal enzyme may be administered, for example, by intravenous injection or intravenous infusion. The administration may be daily, weekly, biweekly, or monthly. The administration dosage may be about 1 mg/kg to about 100 mg/kg body weight, such as about 1 mg/kg to about 50 mg/kg or about 1 mg/kg to about 25 mg/kg body weight. The dosage may be, for example, about 1 mg/kg, about 5 mg/kg, about 10 mg/kg, about 15 mg/kg, about 20 mg/kg, about 25 mg/kg, about 50 mg/kg, or about 75 mg/kg body weight. For example, the glycan-remodeled lysosomal enzyme may be administered by intravenous infusion every two weeks at a dosage of about 20 mg/kg body weight.

As used herein, “subject” or “patient” refers to mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex. In one specific embodiment, a subject is a mammal, preferably a human. In some embodiments, the subject is a human suffering from a disease or disorder needing therapeutic treatment. Suitable subjects may have a disease or disorder, specifically a disease or disorder that can be treated by a protein or enzyme of interest, as described herein.

As used herein, the terms “administering” and “administration” refer to any method of providing a pharmaceutical preparation or composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration and subcutaneous administration. Administration can be continuous or intermittent. In various aspects, a preparation can be administered therapeutically; that is, administered to treat an existing disease or condition. In some embodiments, the administration is intravenous administration.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. In places where ranges of values are given, this disclosure explicitly contemplates other combinations of the lower and upper limits of those ranges that are not explicitly recited. For example, recitation of a value between 1 and 10 or between 2 and 9 also contemplates a value between 1 and 9 or between 2 and 10. Ranges identified as being “between” two values are inclusive of the end-point values. For example, recitation of a value between 1 and 10 includes the values 1 and 10.

Aspects of the present disclosure that are described with respect to methods can be utilized in the context of the compositions of matter or kits discussed in this disclosure. Similarly, aspects of the present disclosure that are described with respect to compositions of matter can be utilized in the context of the methods and kits, and aspects of the present disclosure that are described with respect to kits can be utilized in the context of the methods and compositions of matter.

The scope of the disclosure will be more fully understood upon consideration of the following non-limiting examples.

### EXAMPLES

### Example 1: Chemical Synthesis of Truncated High-Mannose Type Phosphorylated N-Glycan Oxazolines

Following the previously reported synthesis of phosphorylated N-glycans,^{18, 29, 30} selectively protected core disaccharide 15 was synthesized. Starting from D-glucosamine hydrochloride, 11 was prepared in two steps following the reported procedure.³² Compound 11 was then treated with Ag₂CO₃ and BnBr to introduce a benzyl group to the anomeric position, followed by 3-O-benzylation with NaH and BnBr to yield 12 in 75% overall yield. The β-configuration was confirmed by the coupling constant of J_{1,2}=7.8 Hz. Upon regioselective ring-opening of the benzylidene group, 13 was obtained in 81% yield, which was coupled with the known compound 1433 to give the core disaccharide 15 in 69% yield. Next, regioselective ring-opening reaction furnished 16 with a free OH at C6 position, and after the conversion of the 2-azido group into the 2-acetamido group with AcSH,³⁰ the free OH was phosphorylated with dibenzyl N,N-diisopropylphosphoramidite, followed by oxidation with mCPBA¹⁸ to give 18 in 90% yield. Global deprotection of the Bn and PMB groups via a two-step catalytic hydrogenolysis³⁰ gave the free disaccharide 19 in excellent yield. Finally, oxazoline formation was achieved in a single step by treatment with an excess amount of 2-chloro-1,3-dimethylimidazolinium chloride (DMC)³⁴ in water in the presence of Et₃N to afford the phosphorylated core disaccharide oxazoline 5 in 97% yield (Scheme 1).

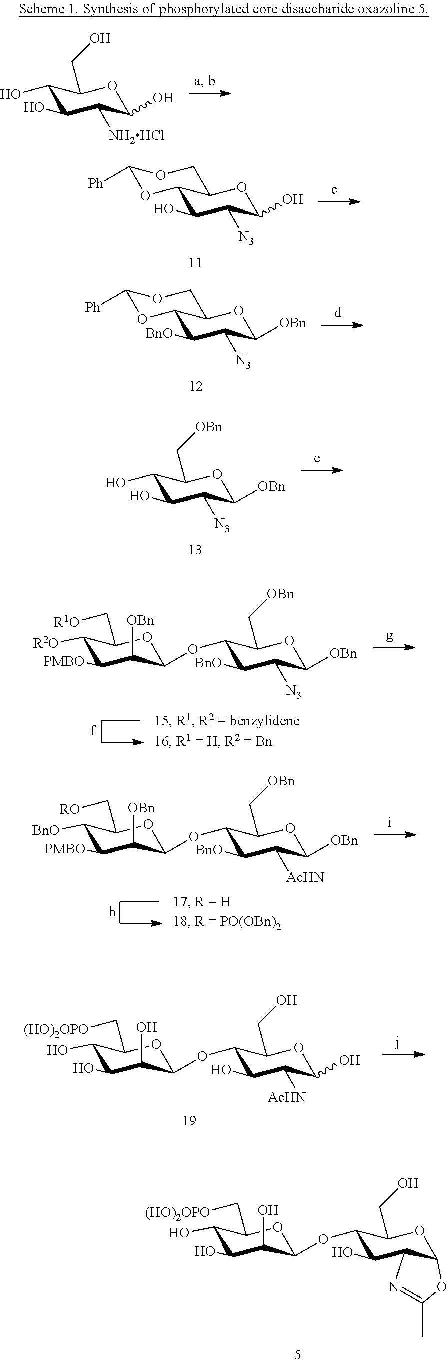

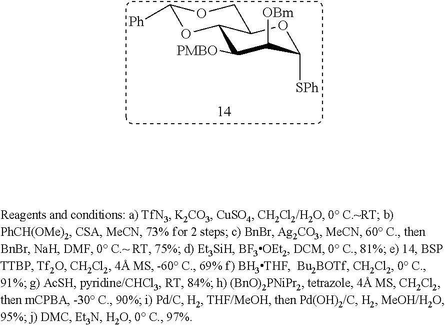

To determine the role of the mannosyl branches, trisaccharide oxazolines 6 and 7 were synthesized with a phosphate group at the α-1,6 or α-1,3-branch, respectively (Scheme 2). Deprotection of the 3-O-PMB in 15 gave 20 as an acceptor, then 16 and 20 were glycosylated with glycosyl donor 21³⁵ to give trisaccharides 23 and 24, respectively. A two-step manipulation was conducted to convert the benzoyl group to permanent benzyl group, giving 26 in 90% yield. However, the benzylation step failed to afford 25 even under harsh conditions probably due to high steric hindrance, thus the 2-O-Bn imidate 22³⁶ was used as the donor, which furnished 25 in 72% yield along with 15% of the β isomer. Next, upon the reduction of the azido group to the acetamido group followed by the selective deprotection of the TIPS group with TBAF, 29 and 30 were obtained in 85% and 87% yield, respectively, which were ready for phosphorylation. Finally, the phosphate group was introduced at the C6 position to give the fully protected derivatives 31 and 32 in 75% and 90% yield, respectively. Global deprotection via hydrogenolysis removed all the Bn, PMB and benzylidene groups simultaneously, giving free trisaccharides 33 and 34, which were converted into glycan oxazolines 6 and 7 respectively by reaction with DMC in a single step.

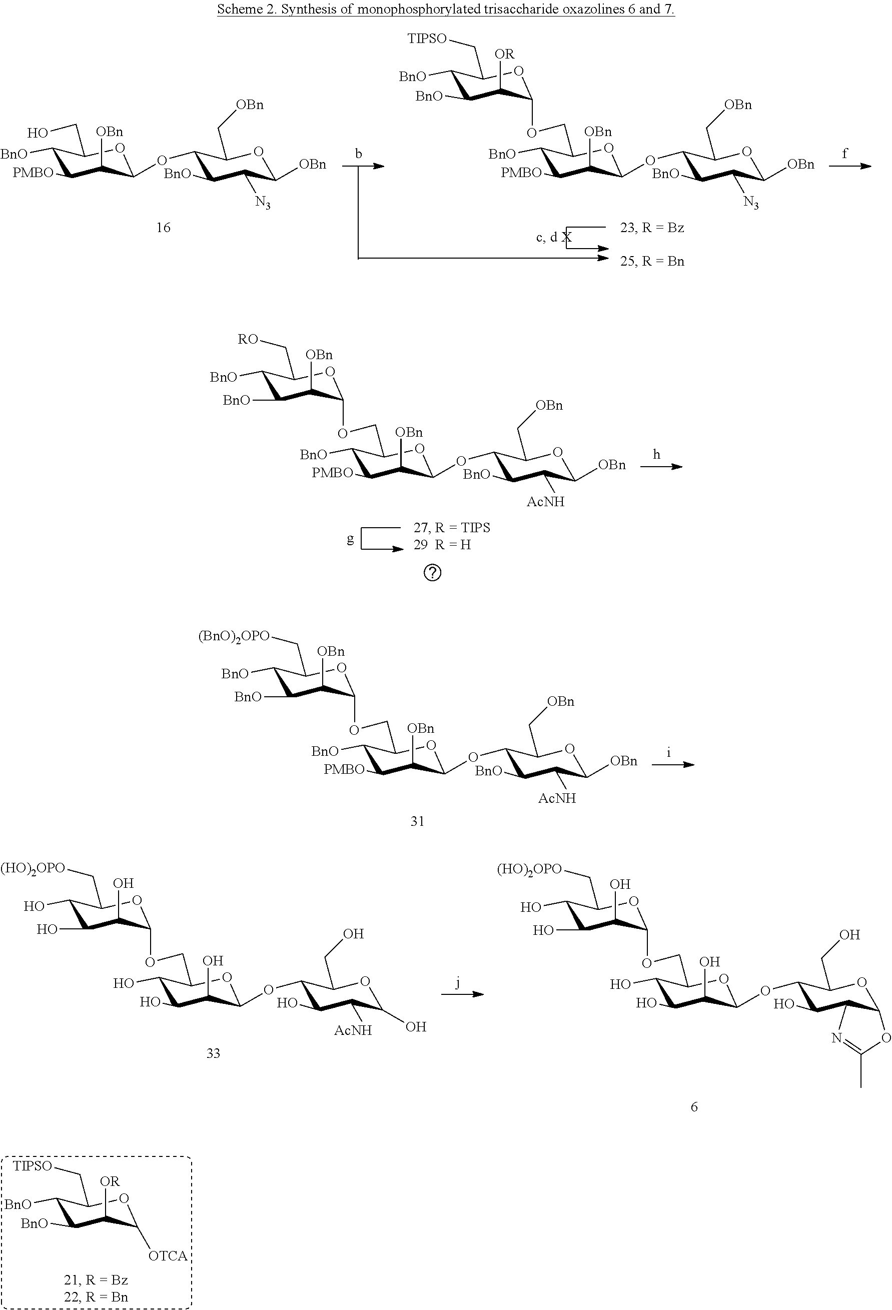

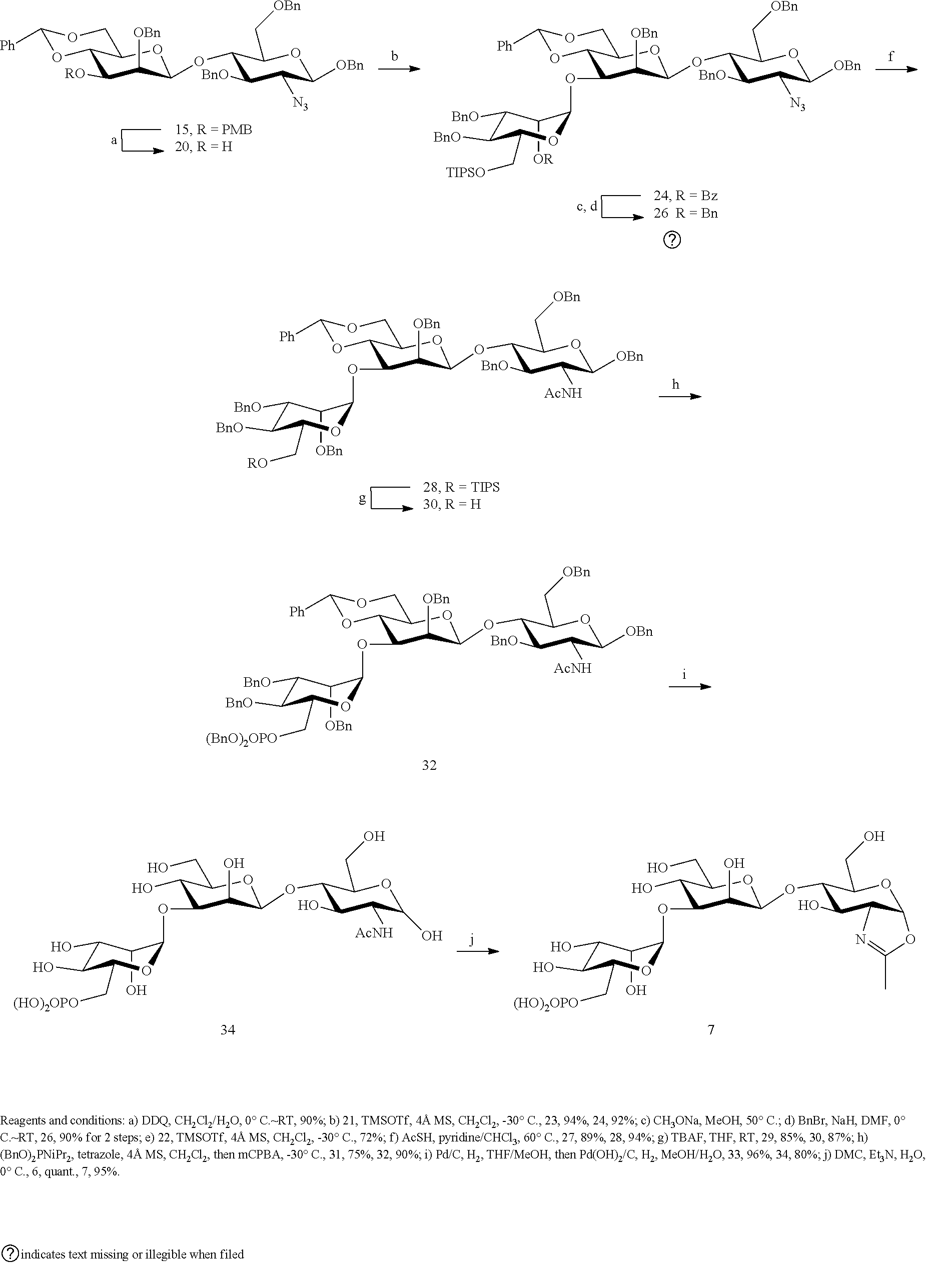

For the synthesis of tetrasaccharides with only α-1,3 or α-1,6-branch, the disaccharide 37 was first synthesized by coupling of donor 21 with the known acceptor 36.37 After the conversion of the benzoyl group to permanent benzyl group, the resulting disaccharide 38 was then used as the glycosyl donor for coupling with 20 under the promotion of NIS and TfOH to provide 39 in 74% yield, which was confirmed as the sole a isomer. After reduction of the azido group to acetamido group, followed by the selective deprotection of TIPS group, the C6 free OH was phosphorylated to give 42. Finally, the global deprotection and oxazoline formation provided the oxazoline 8 in good yield (Scheme 3). In parallel, the 1,6-linked disaccharide 45 was prepared by the coupling of donor 21 with 44,³⁸ which furnished the glycosyl donor 46 upon the conversion of the benzoyl group to benzyl group. The [2+2] coupling of 46 and 16 afforded 47 in 72% yield along with 17% of β isomer. Conversion of the azido group to acetamido group, followed by deprotection of TIPS and standard phosphorylation gave 50 in good yield. Finally, global deprotection of 50 followed by DMC treatment gave the oxazoline 9 (Scheme 3).

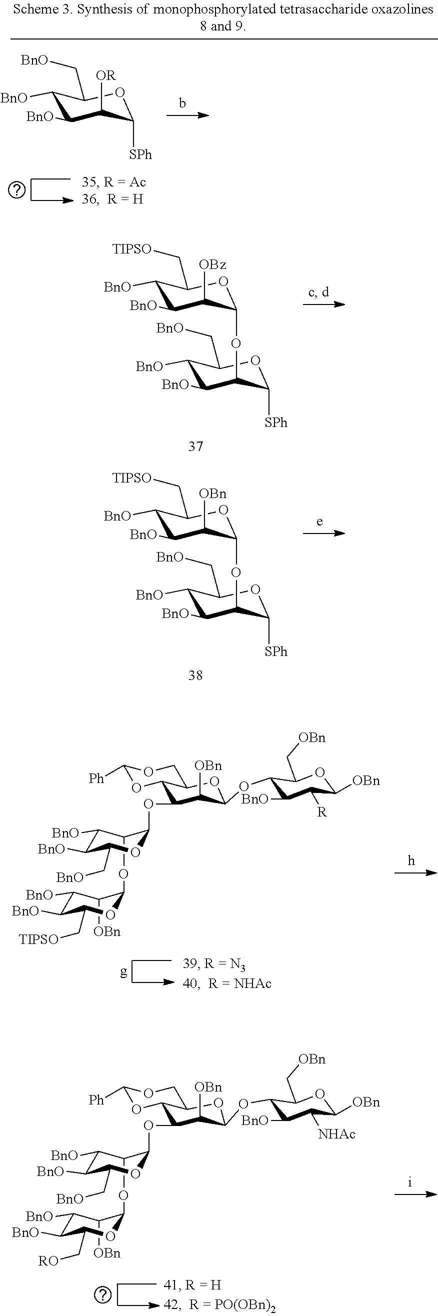

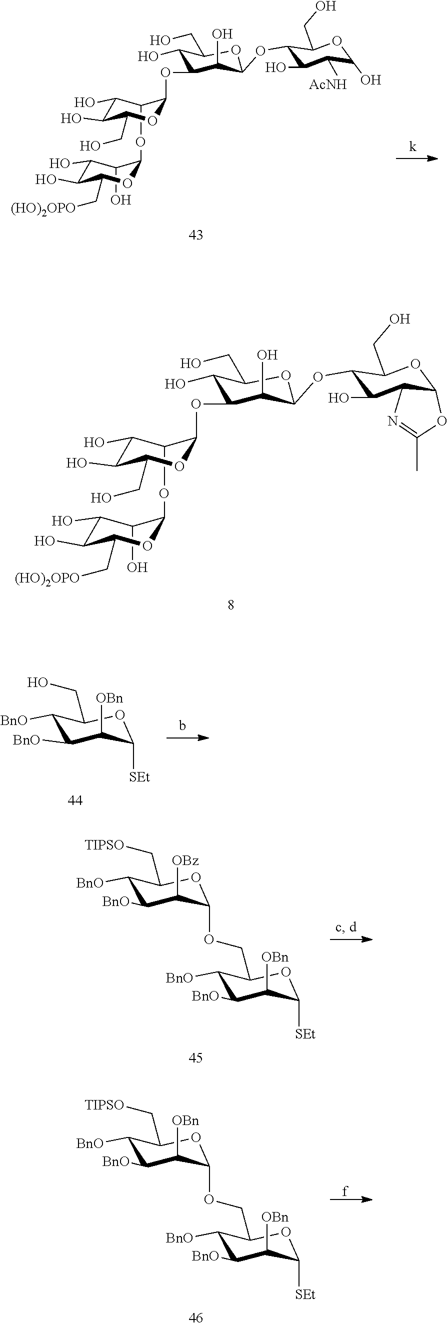

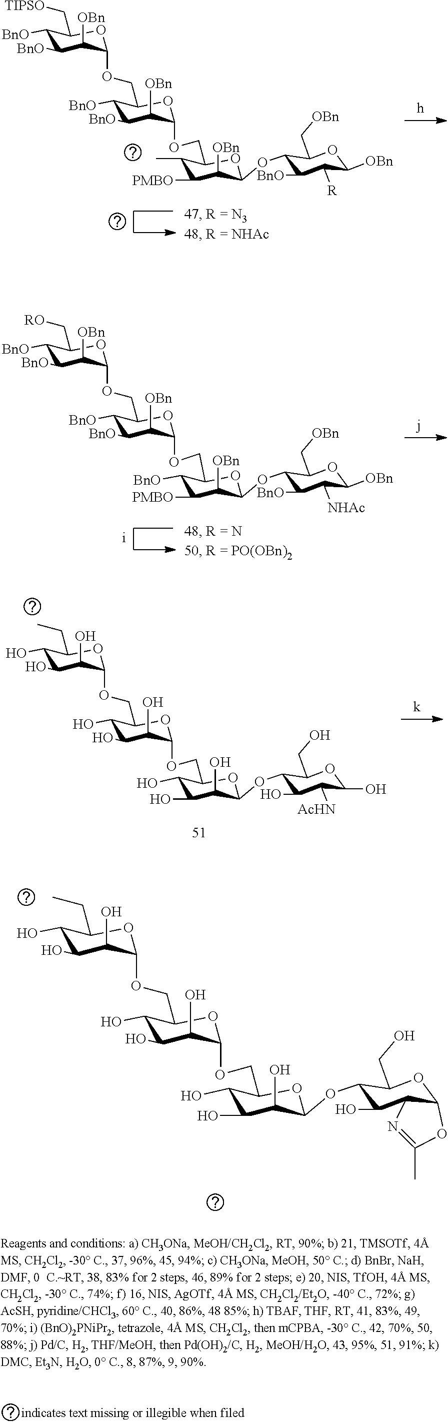

Previous studies have shown that not only the glycan structure determinants but also its optimal orientation is important for high-affinity binding with CI-MPR.^{17, 39, 40} Considering the critical role of the Man3 core for retaining the glycan conformation, the pentasaccharide oxazoline 10 was designed and synthesized. Starting with tetrasaccharide 39, regioselective ring-opening reaction afforded 52 with a C6 free OH at the core mannosyl residue, then another mannosyl residue was installed at this position using glycosyl donor 3537 to give 53 in 80% yield. After the acetyl group was converted into benzyl group, oxazoline 10 was readily obtained as described for the synthesis of 5˜9 by sequential reduction of azido group, deprotection of TIPS, phosphorylation, global deprotection and final oxazoline formation (Scheme 4).

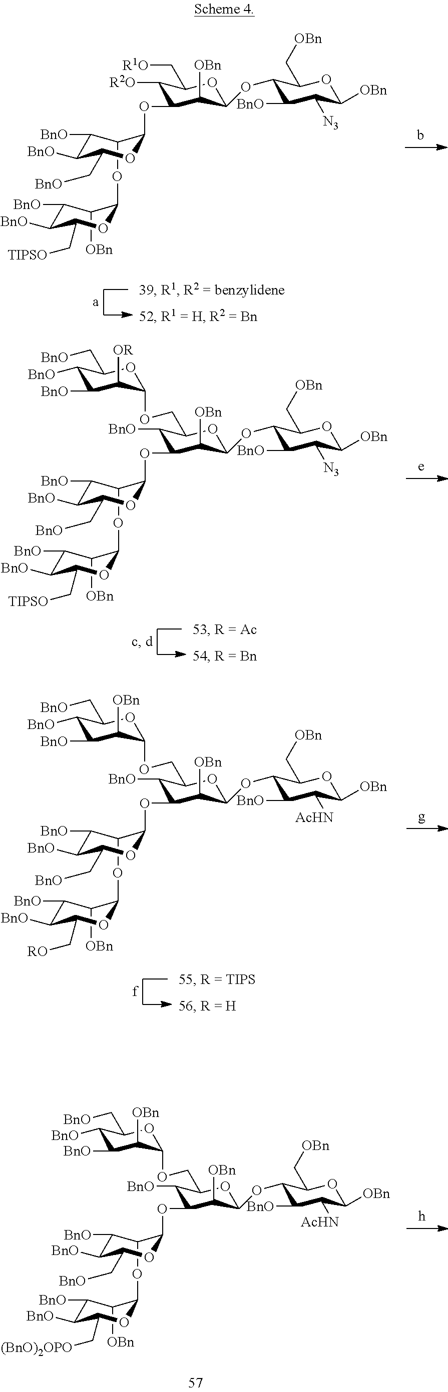

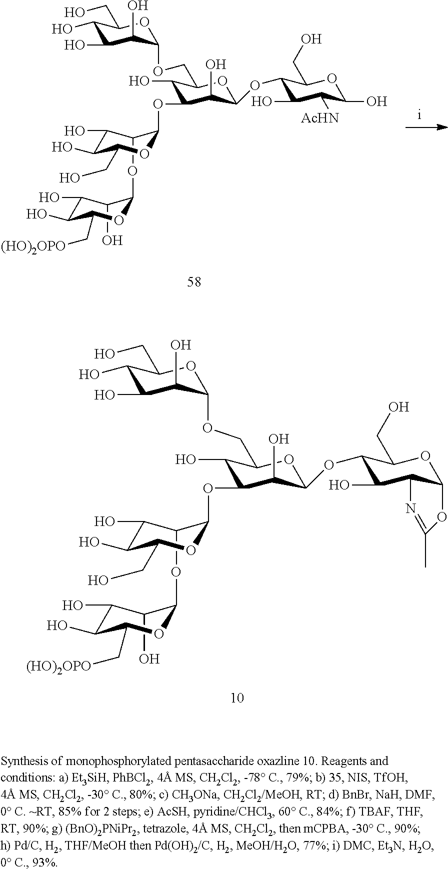

### Example 2: Evaluation of the Synthetic M6P-Glycan Oxazolines as Donor Substrates for Enzymatic Transglycosylation

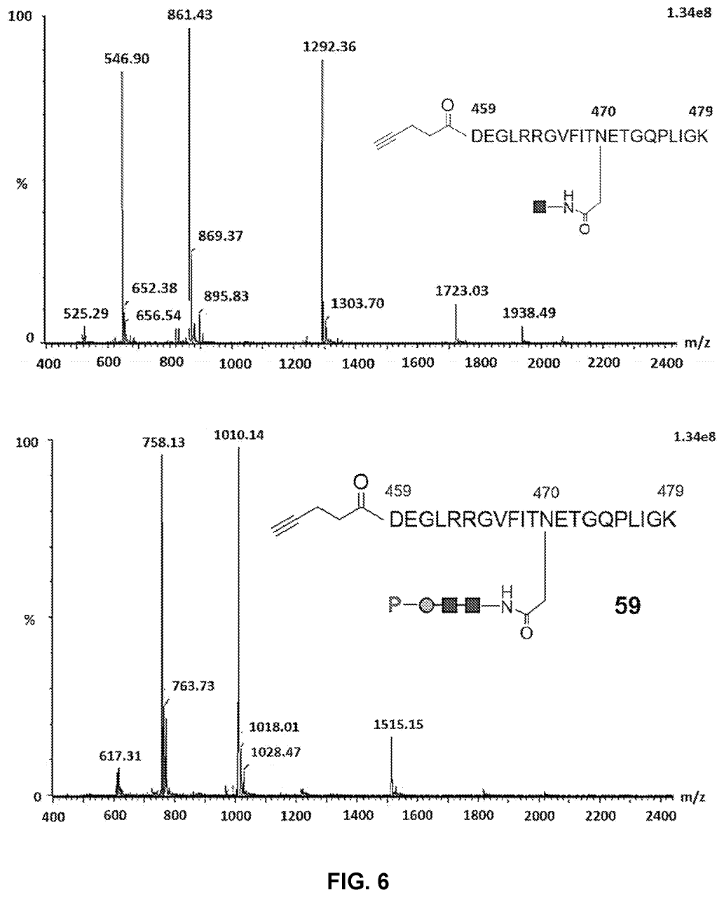

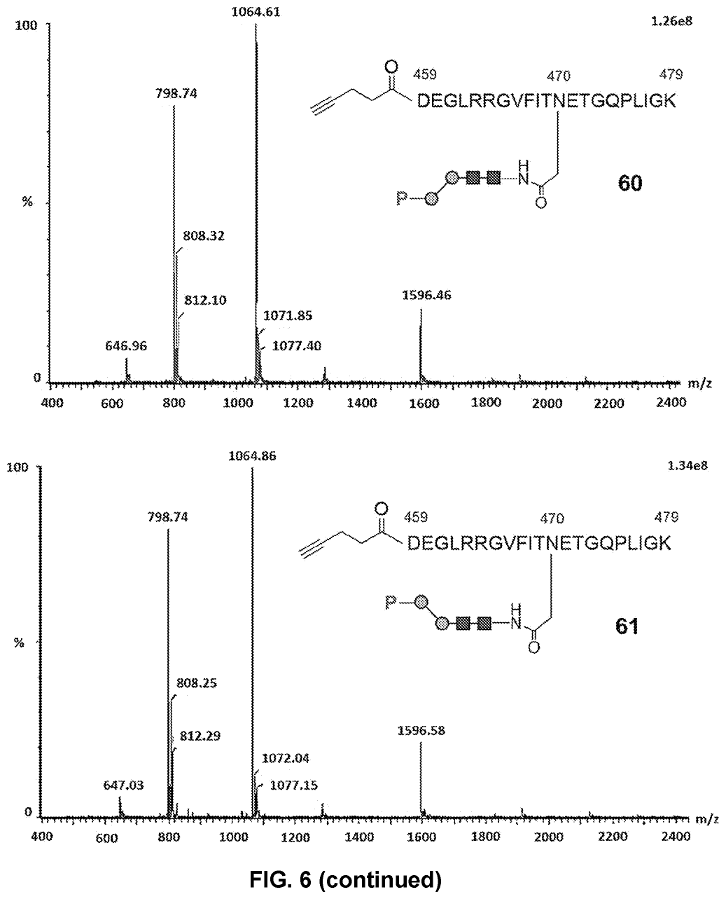

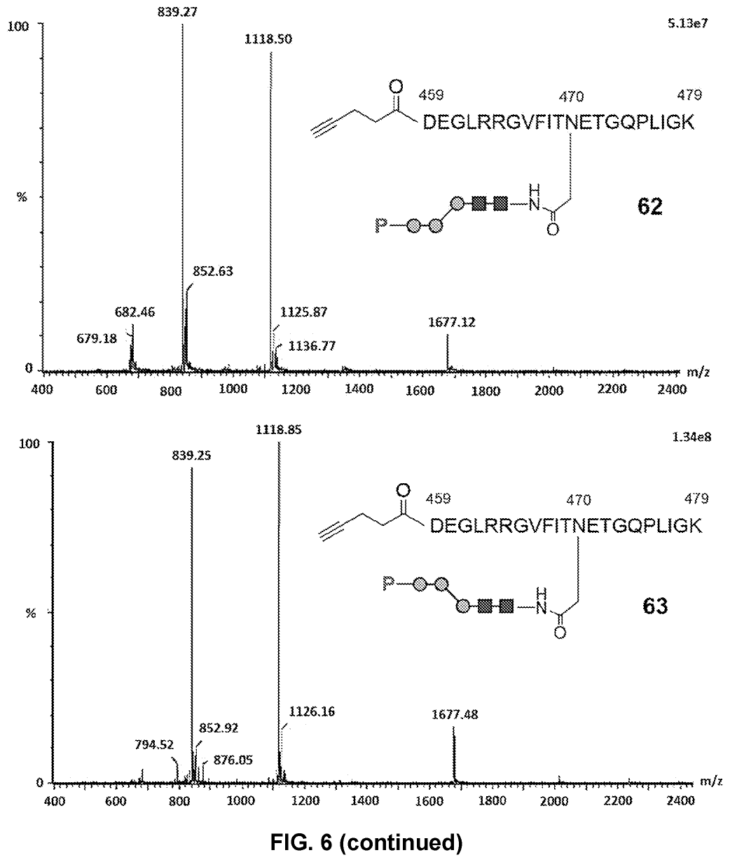

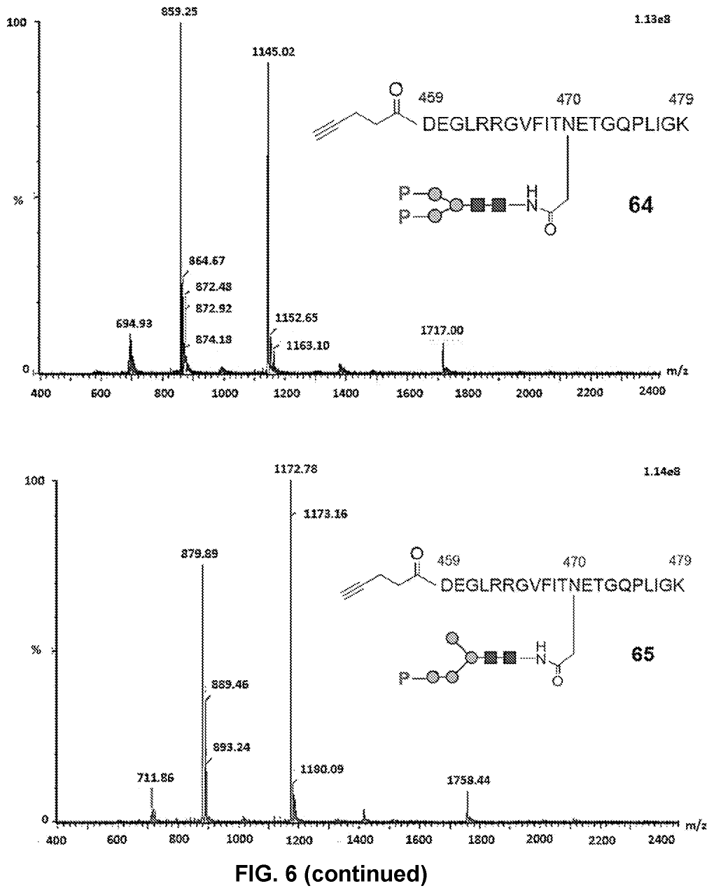

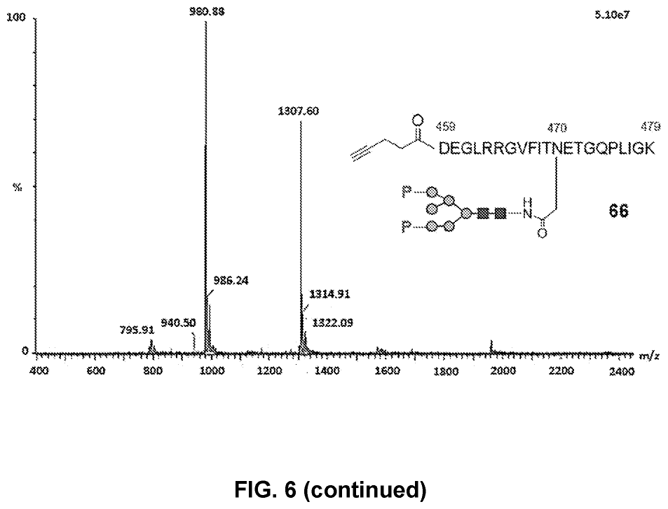

The activities of the synthetic phosphorylated oxazolines as donor substrates for enzymatic transglycosylation were tested. For that purpose, a 21-mer glycopeptide (aa 459-479) derived from rhGAA was selected as a model sequence, in which the N470 residue was selected to install an M6P-glycan,¹⁶ and the precursor GlcNAc-peptide was synthesized via automated solid-phase peptide synthesis (SPPS). Previous studies have shown that wild-type Endo-A, an endoglycosidase from Arthrobacter protophormiae,⁴¹ is efficient for the transglycosylation of truncated phosphorylated Man3GlcNAc oxazoline to GlcNAc-peptides,^{29, 30} but not suitable for transferring large natural M6P high-mannose N-glycan oxazolines, due to its rapid hydrolysis of both the oxazoline donors and the resulting transglycosylation products.³⁰ In this study, the truncated structures (4˜10) acted as good substrates of wild-type Endo-A, and the resulting products, once formed, were barely hydrolyzed by the enzyme, affording the desired glycopeptides (59˜65) in good isolated yields. The newly formed phosphorylated products were eluted later than the GlcNAc-peptide under the reverse-phase HPLC condition, and the identities of the products were confirmed with ESI-MS (FIG. 6). Notably, the transglycosylation yields could be driven to 80%˜90% if additional sugar oxazolines were added to the reaction. Finally, for the bis-phosphorylated ManGlcNAc oxazoline 1, Endo-A-N171A was used and the glycopeptide 66 was obtained in 53% yield after purification by HPLC (Scheme 5).

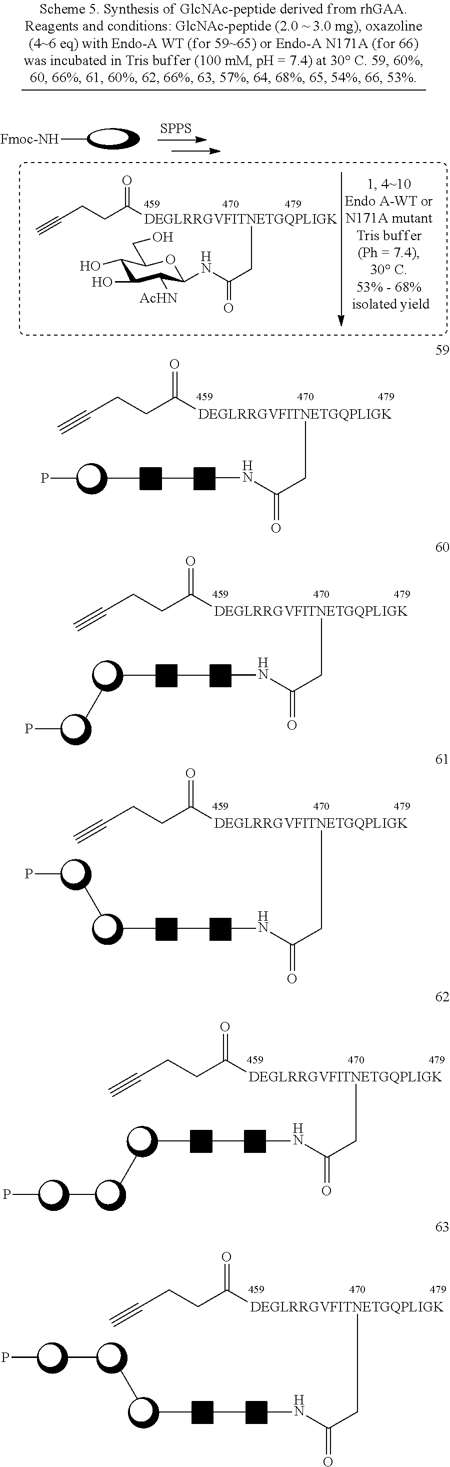

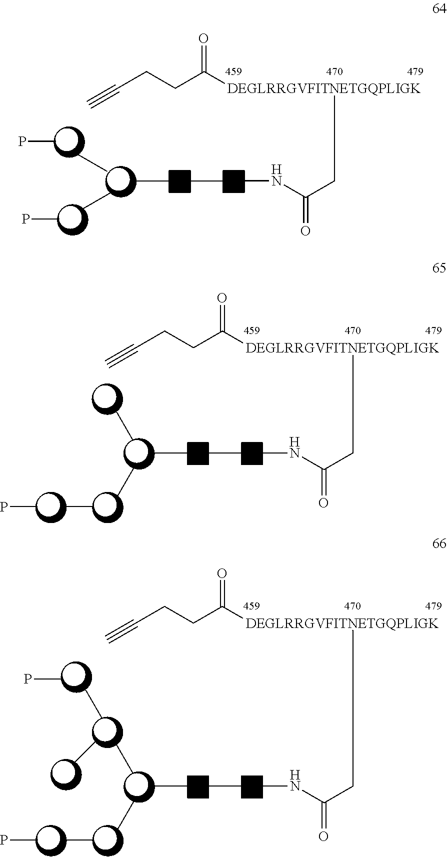

### Example 3: Evaluation of the Affinity of the Synthetic M6P-Glycopeptides for the M6P Receptor (CI-MPR)

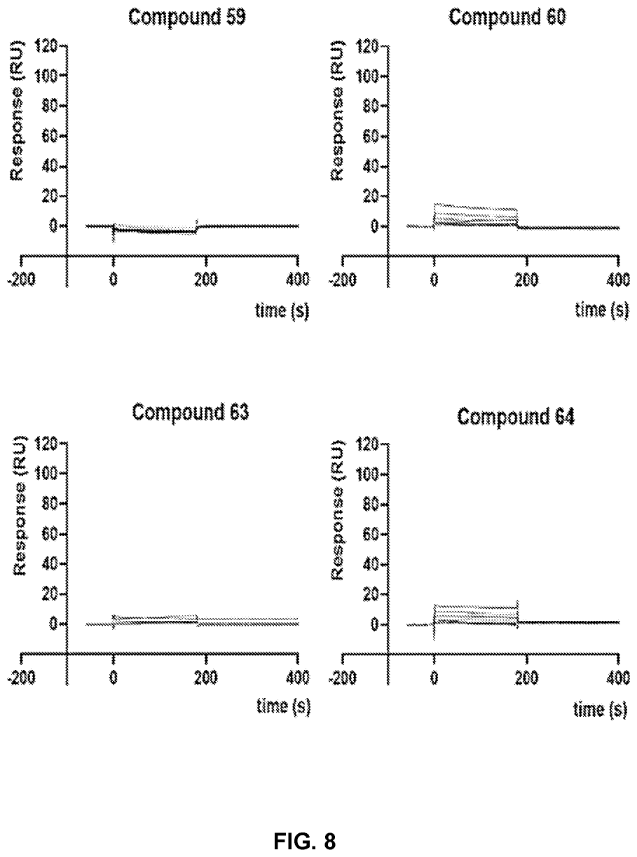

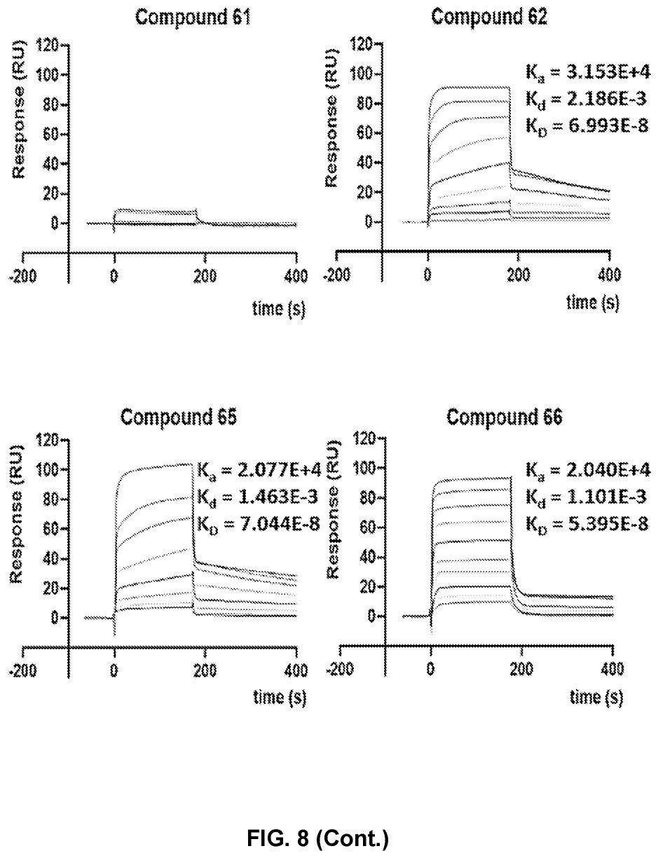

To determine the minimal M6P glycan structures that can still provide high-affinity for CI-MPR, SPR binding studies were performed with immobilized CI-MPR (FIG. 8 and Table 1). The results revealed that the glycopeptide 62 derived from tetrasaccharide oxazoline 8 with a Man6P-α1,2-Man disaccharide moiety at α1,3-arm of the core β-mannose residue showed strong binding affinity (70 nM) with CI-MPR, which was comparable to that of glycopeptide 66 with a bis-phosphorylated Man₆GlcNAc₂ ligand (54 nM). The glycopeptide 65 obtained from the pentasaccharide oxazoline 10 showed similar affinity (82 nM), suggesting that the presence of an additional α1,6-linked mannose moiety did not affect the affinity. Interestingly, when the M6P moiety was α1,3- or α1,6-linked to the next mannosyl residue in the N-glycan, the resulting glycopeptides (60, 61 and 63) showed dramatically decreased affinity for CI-MPR. Taken together, the experimental data suggest that tetrasaccharide oxazoline 8 with a Man6P-α1,2-Man disaccharide moiety at α1,3-arm of the core β-mannose residue appears to be the minimal truncated N-glycan oxazoline derived from the natural structure to provide a high-affinity ligand for CI-MPR. The results further suggest that a Man6P-α1,2-Man disaccharide moiety constitutes an essential structural motif that retains strong binding affinity for the receptor.^{39, 40}

TABLE 1
SPR analysis of the binding of M6P-containing glycopeptides
with the immobilized CI-MPR receptor. ^{[a]}

| Compounds | 59 | 60 | 61 | 62 | 63 | 64 | 65 | 66 |
|---|---|---|---|---|---|---|---|---|
| K_{D} (μM) | >50 ^{[b]} | 1.8 ^{[c]} | >50 ^{[b]} | 0.070 ± | >50 ^{[b]} | 0.84 ^{[c]} | 0.082 ± | 0.054 ± |
| | | | | 0.002 | | | 0.014 | 0.006 |

^{[a]} Serial 2-fold dilution of concentrations (7.8-4000 nM) were performed for the SPR analysis.
^{[b]} No obvious binding was detected up to 50 μM.
^{[c]} Estimated by steady state fitting because the kinetic fitting did not give reliable data. The standard deviations were obtained from three independent experiments.

### Example 4: Glycan Remodeling of RNase B and the Affinity of the Glycan Remodeled Protein for CI-MPR

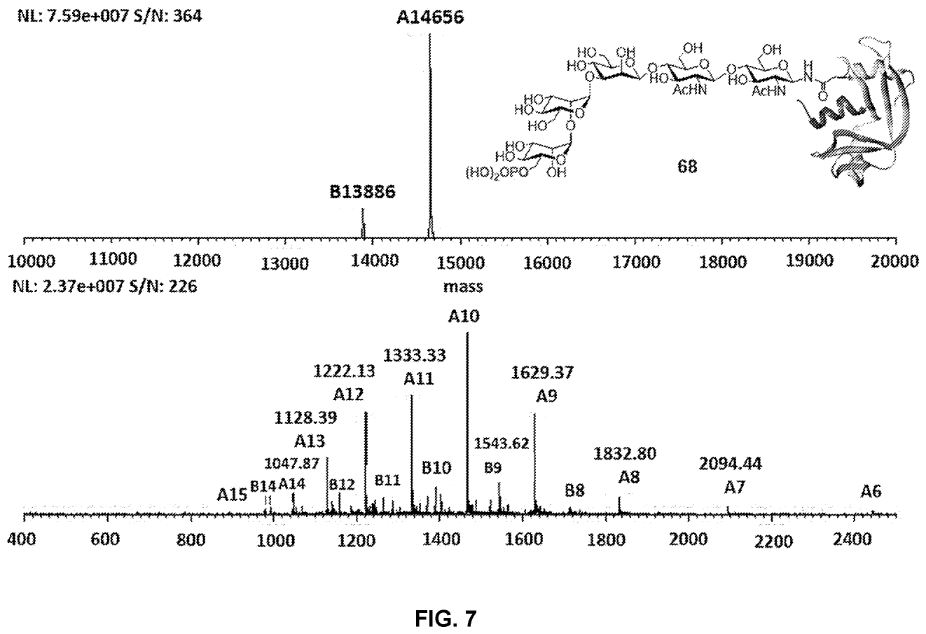

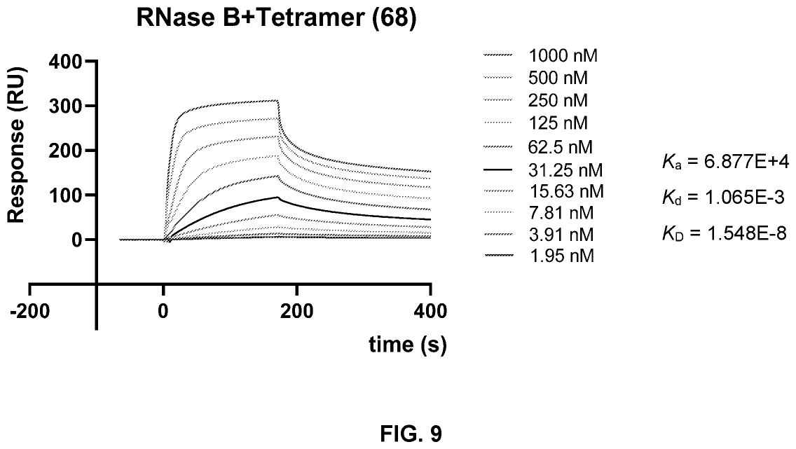

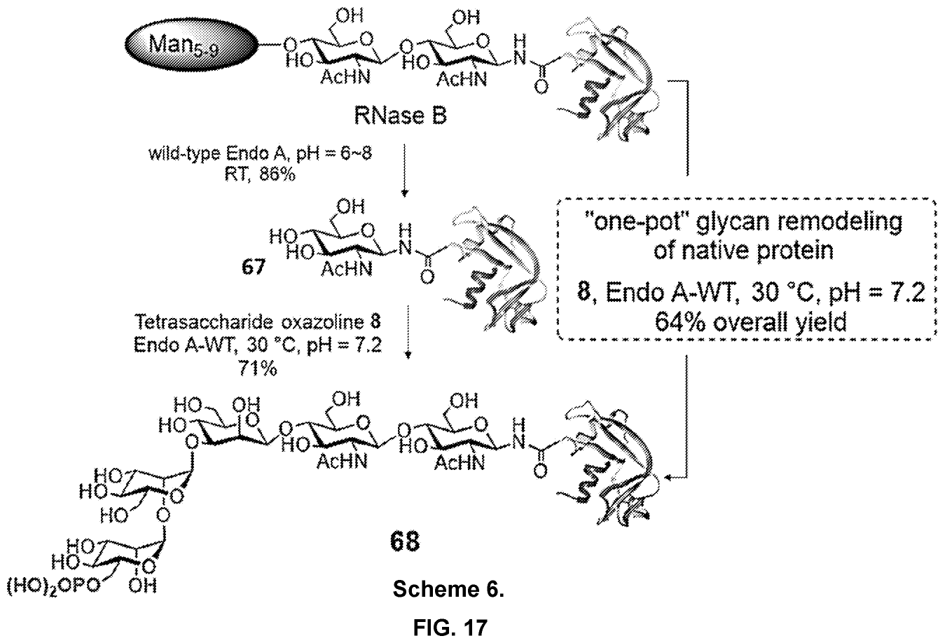

With the identification of the minimal high-affinity ligand (8), the suitability of this M6P glycan oxazoline was next investigated for glycan remodeling of glycoproteins, first using bovine RNase B as a model substrate, which carries a high-mannose type N-glycan at a single N-glycosylation site (Asn-34) (Scheme 6). RNase B was deglycosylated with wild-type Endo-A to give the homogeneous GlcNAc-RNase B (67). Then the enzymatic reaction between oxazoline 8 and GlcNAc-RNase B under the catalysis of the same enzyme smoothly afforded the phosphorylated glycoprotein 68 in 71% yield, the identity of which was confirmed by ESI-MS (calculated, M=14655; found, M=14656, deconvolution data, FIG. 7). Although wild-type Endo-A was quite active for hydrolyzing natural Man5-Man9 N-glycan structures, it was found that the M6P glycan oxazoline (8) was only very slowly hydrolyzed by this enzyme, and the resulting glycoprotein was resistant to hydrolysis under the reaction conditions, probably due to the truncation and phosphorylation of the N-glycan. The huge difference in the hydrolytic activities of Endo-A toward the starting glycoprotein and the resulting product, together with its excellent transglycosylation activity toward the truncated M6P-glycan oxazoline, prompted us to devise a one-pot strategy for enzymatic glycan remodeling. Thus, RNase B was incubated with wild-type Endo-A at 30 ºC for 30 min before the addition of oxazoline 8 and the reaction mixture was incubated until the transglycosylation was complete. It was found that the deglycosylation of RNase B proceeded quickly, followed by the formation of the desired phosphorylated glycoprotein 68 as a result of transglycosylation. The one-pot protocol simplified the procedure by omitting the purification of the intermediate after deglycosylation, thus improving the overall efficiency of the glycan remodeling approach. SPR binding experiments revealed that the M6P-containing RNase B (68) showed strong affinity for CI-MPR (K_{D}=15.8 nM, FIG. 9), which, again, was comparable to that of the glycoprotein with a large bis-M6P-Man₆GlcNAc₂ glycan.³⁰ The results suggest that a simple M6P tetrasaccharide oxazoline is sufficient to enable the glycan remodeling of glycoprotein with high-affinity binding to CI-MPR in a one-pot manner. See FIG. 17, scheme 6.

### Example 5: One-Pot and Glycan-Selective Remodeling of the Recombinant Human Acid α-Glucosidase (rhGAA, Lumizyme) Using Wild-Type Endo-A and Endo-F3

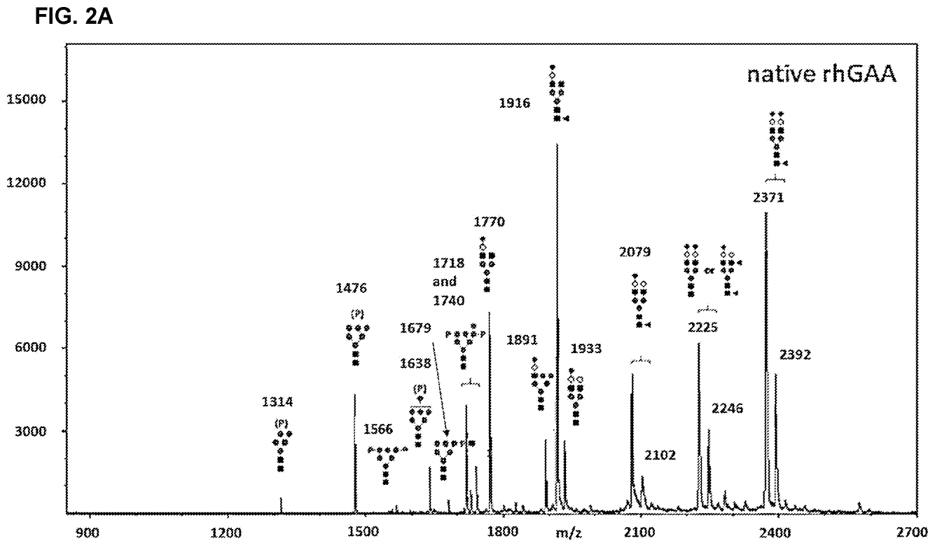

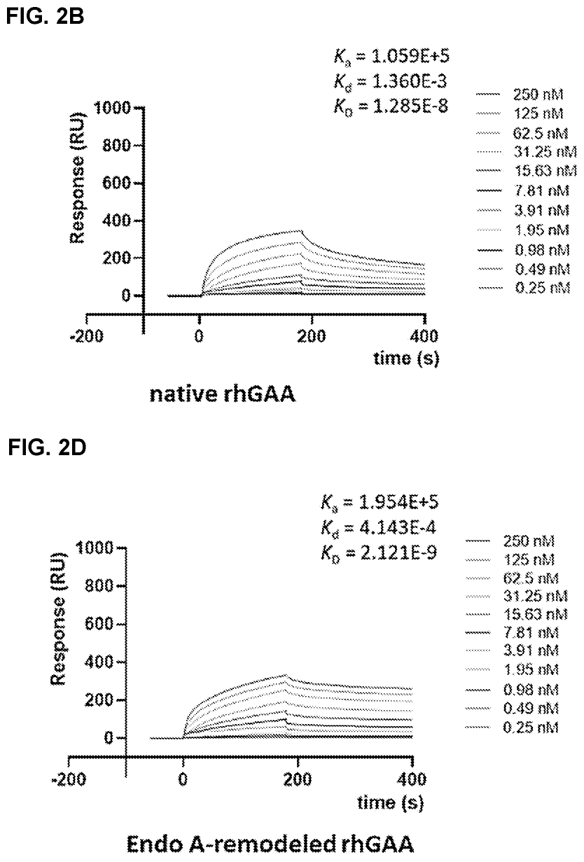

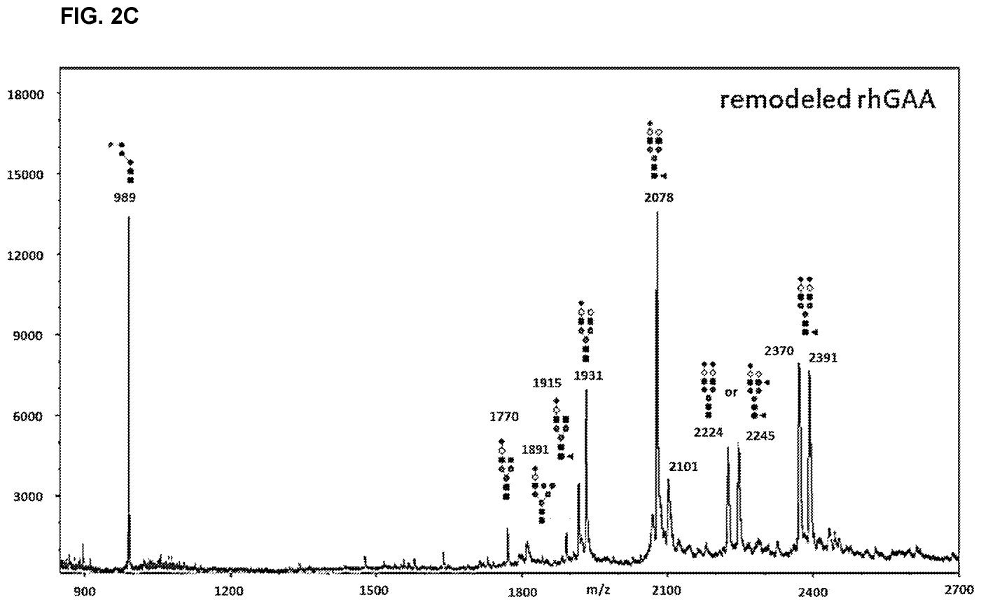

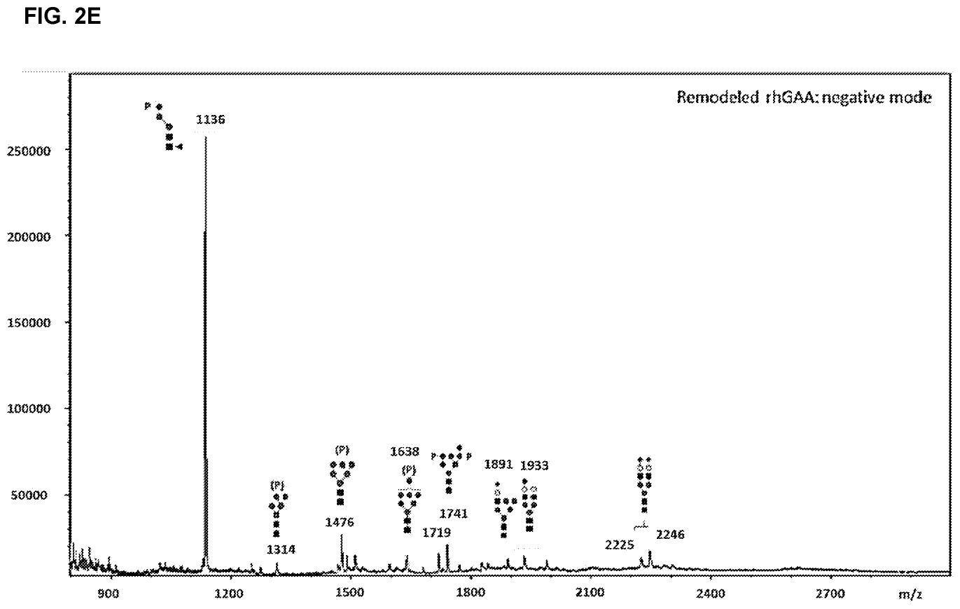

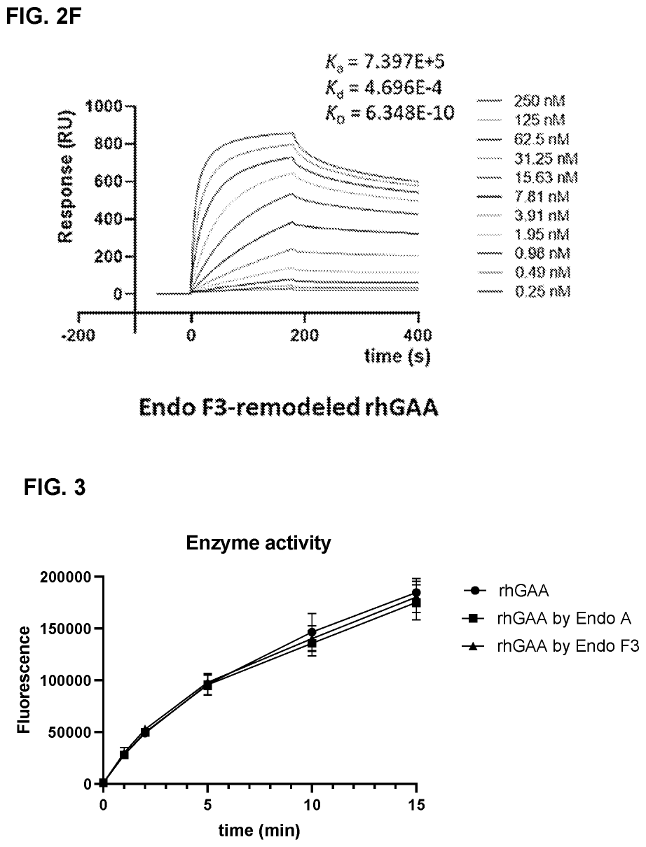

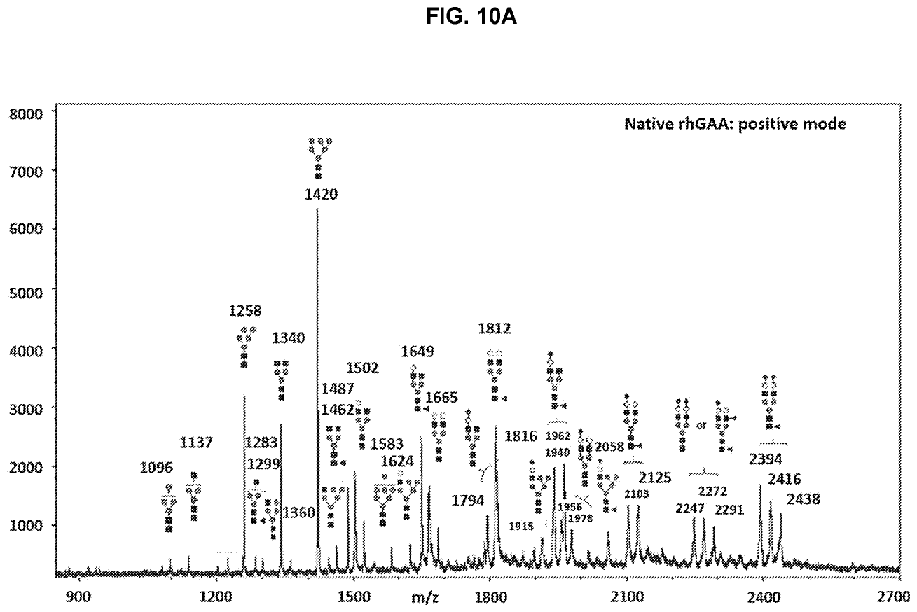

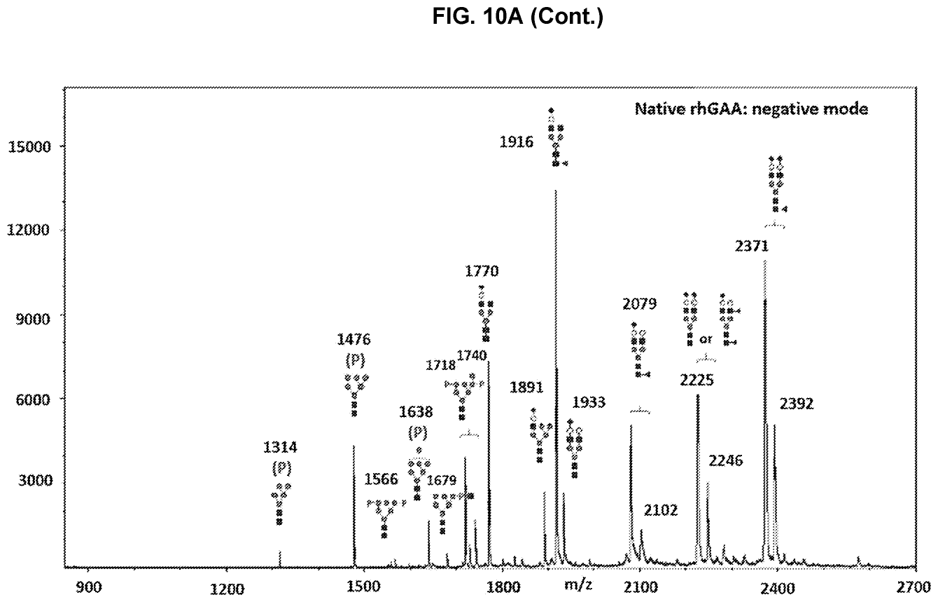

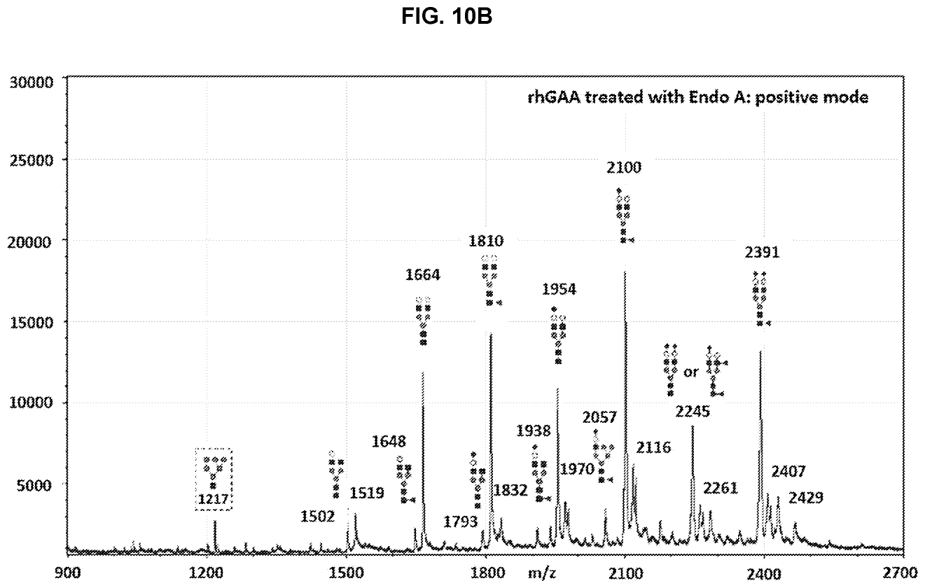

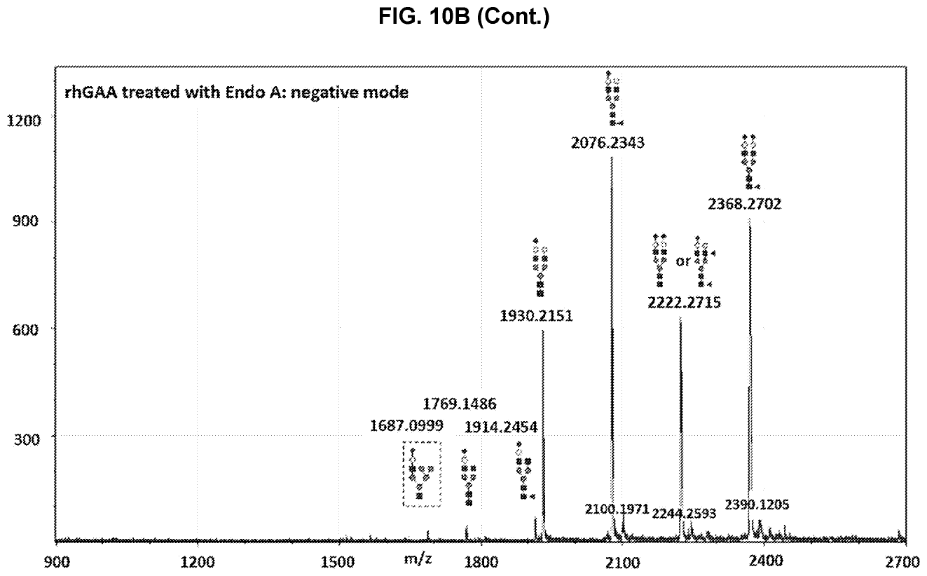

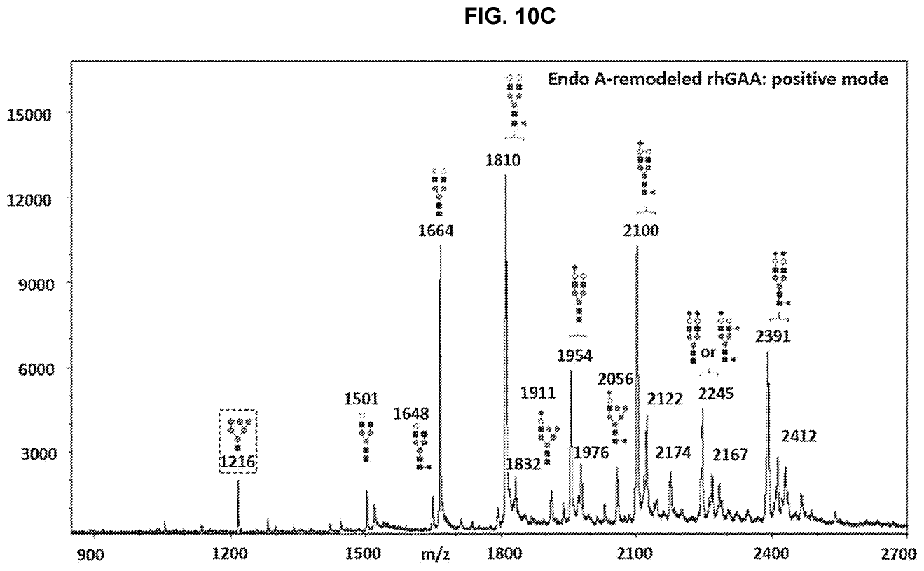

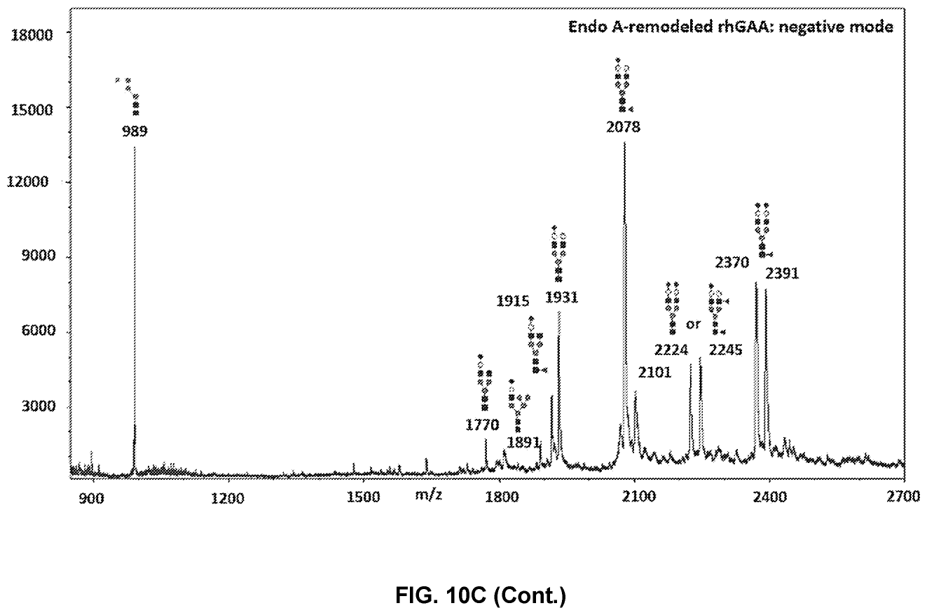

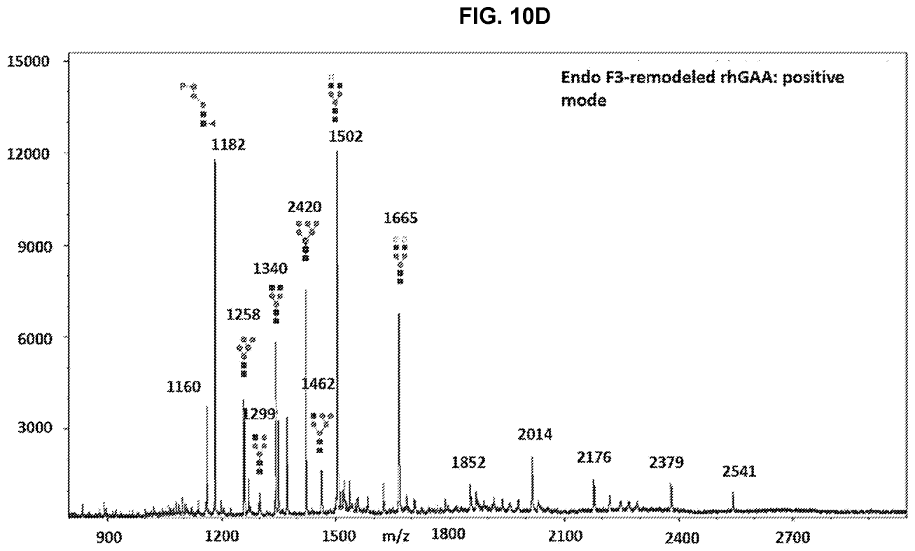

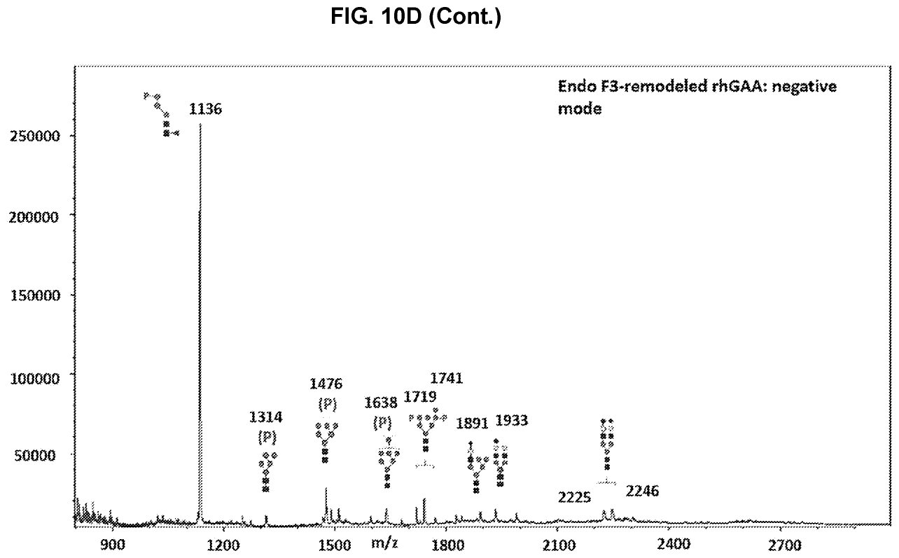

Following the promising model study with RNase B, further studies were performed to evaluate the feasibility of the one-pot enzymatic M6P-glycan remodeling on the recombinant human acid α-glucosidase (rhGAA; Lumizyme, Sanofi Genzyme), a therapeutic, multiply glycosylated lysosomal enzyme used for the treatment of Pompe disease.²² rhGAA produced in CHO cells has seven N-glycosylation sites, 31 of which two sites (N233 and N470) reportedly contain high-mannose type glycans, while the rest are mainly occupied by core-fucosylated complex type N-glycans.¹⁹ Lumizyme, which is currently used for the enzyme replacement therapy of Pompe disease, contains relatively low amounts of high-mannose M6P-glycans, thus limiting its targeting and the overall therapeutic efficacy. The low M6P contents partially explain why up to 20-fold higher dose is usually required for the treatment of Pompe disease than those of the lysosomal enzymes used for the treatment of other LSDs.²² Considering the substrate specificity of different endoglycosidases, studies were designed to selectively modify the high-mannose type N-glycans but leave the complex type N-glycans unchanged, or keep the high-mannose type N-glycans while acting on the complex type N-glycans selectively. Based on the success in modification of RNase B, wild-type Endo-A offers an excellent choice to selectively trim the high-mannose type glycans and install simultaneously the synthetic M6P-glycan via a one-pot strategy in view of its substrate specificity.⁴¹ Thus, the commercial rhGAA was treated with Endo-A, followed by addition of several portions of oxazoline 8 at 30° C. in one pot. The resulting reaction mixture was treated with Glutathione Agarose to remove the GST-tagged Endo-A, and the cleaved glycans and salts were removed by ultrafiltration (Scheme 7). Glycan analysis revealed the removal of high-mannose type N-glycans and introduction of the M6P-glycan after transglycosylation without affecting the complex type N-glycans (FIG. 2A, FIG. 2C, and FIG. 10). This result confirmed the efficiency of the site-selective glycan remodeling of a multiply glycosylated therapeutic protein.

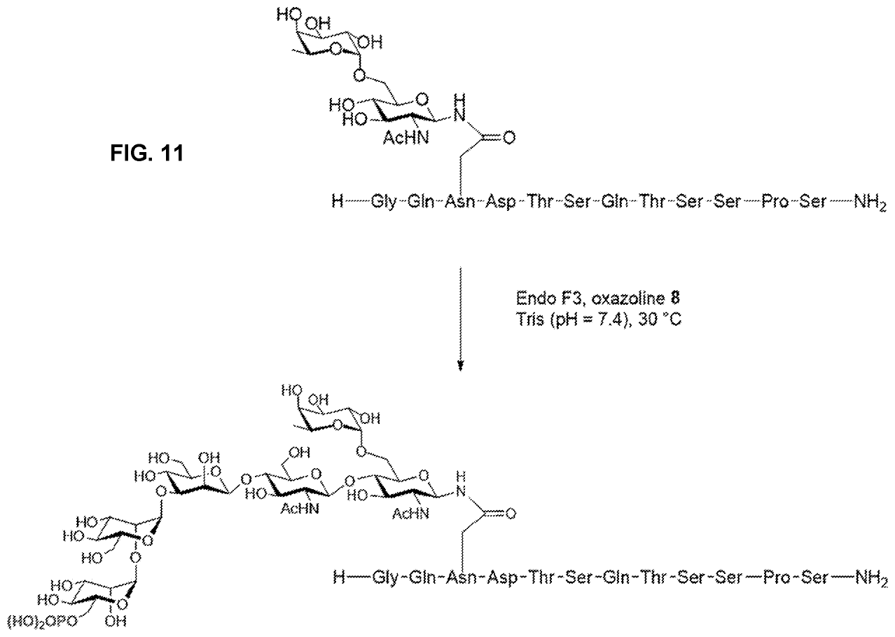

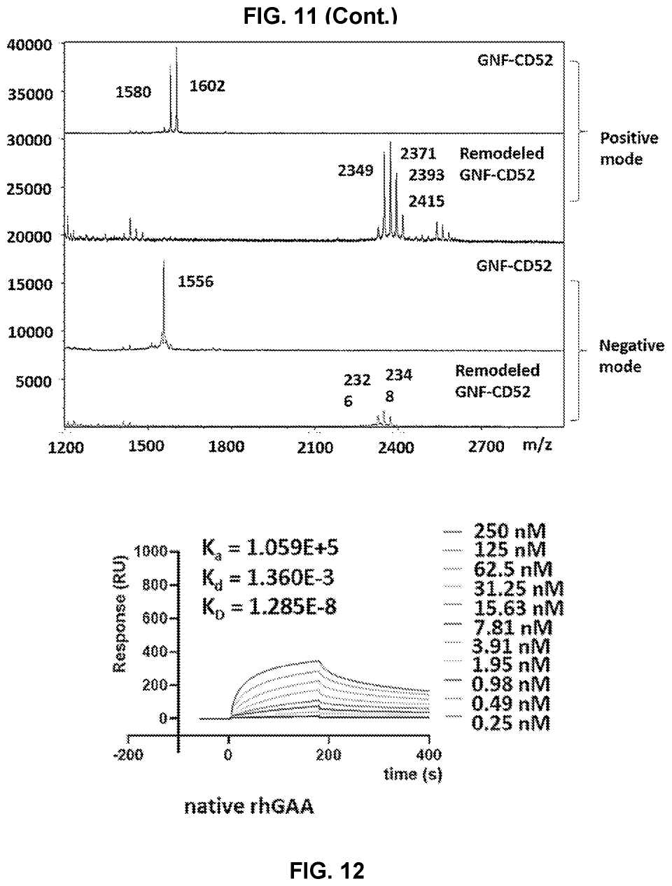

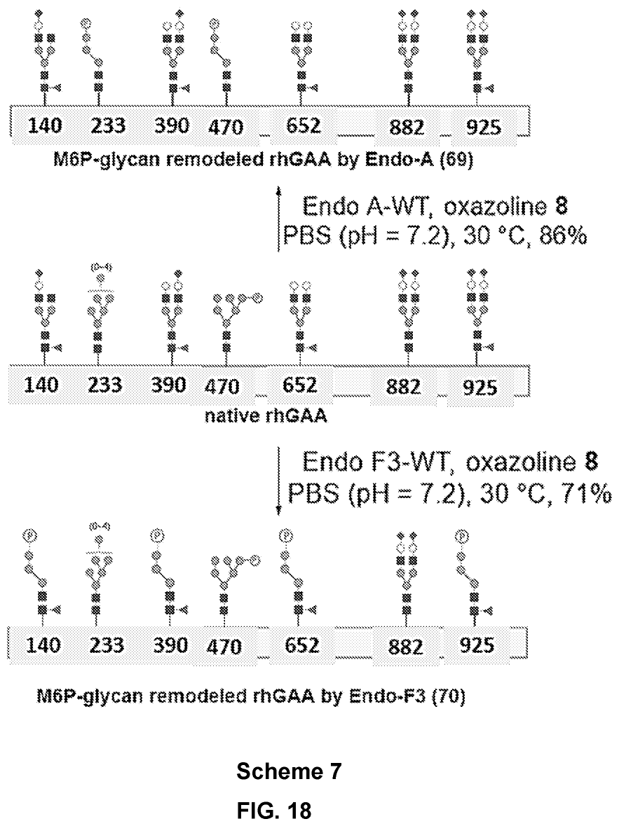

Since the major glycoforms of rhGAA are core-fucosylated complex type N-glycans, studies were performed to selectively remodel the core-fucosylated complex type N-glycans with M6P-glycan and to keep the original phosphorylated high-mannose type N-glycans unchanged. For this purpose, these studies employed Endo-F3, an endoglycosidase from Elizabethkingia meningoseptica that efficiently hydrolyzes core-fucosylated complex type N-glycans but is unable to cleave high-mannose type N-glycans.⁴²⁻⁴⁵ As an initial experiment, Endo-F3 indeed efficiently transferred the minimal M6P-tetrasaccharide oxazoline (8) to a model core-fucosylated GlcNAc-peptide (Fuca1,6GlcNAc-CD52) and, interestingly, the resulting M6P-glycopeptide was resistant to hydrolysis by this enzyme (FIG. 11). This preliminary study prompted us to test a one-pot strategy for selective glycan remodeling of rhGAA with Endo-F3. Thus, the commercial rhGAA was treated with Endo-F3 to selectively remove the core-fucosylated N-glycans, presumably at the N140, N390, N652, and N925 sites based on previous site-specific glycosylation profiling analysis,^{16, 19} followed by three portions of tetrasaccharide oxazoline 8 every 2 hours at 30° C. (Scheme 7). The resulting reaction mixture was purified by HisTrap column to remove the His-tagged Endo-F3, and the cleaved glycans and extra salts were removed by ultrafiltration. Notably, Endo-F3-deglycosylated rhGAA was unstable and prone to precipitation. As a result, the one-pot simultaneous deglycosylation/transglycosylation strategy appeared to be much more efficient than a stepwise method that required an isolation of the deglycosylated intermediate. Glycan analysis confirmed the removal of complex type N-glycans and introduction of the M6P-glycan without affecting the high-mannose type N-glycans (FIG. 2E and FIG. 10). The Endo-F3-based glycan remodeling method is complementary to the Endo-A-based glycan remodeling in terms of the N-glycan selectivity. Given the fact that most of the therapeutic enzymes used in ERTs are multiply glycosylated and usually carry both high-mannose type and complex type N-glycans, the present selective glycan remodeling method provides a general platform for a single-step, site- and glycan-selective M6P-glycan remodeling of most lysosomal enzymes with a more homogeneous product, which appears to be superior to the existing chemical conjugations with natural and synthetic M6P ligands.^{16, 24} See FIG. 18, scheme 7.

### Example 6: CI-MPR Binding of the M6P Glycan-Remodeled rhGAA

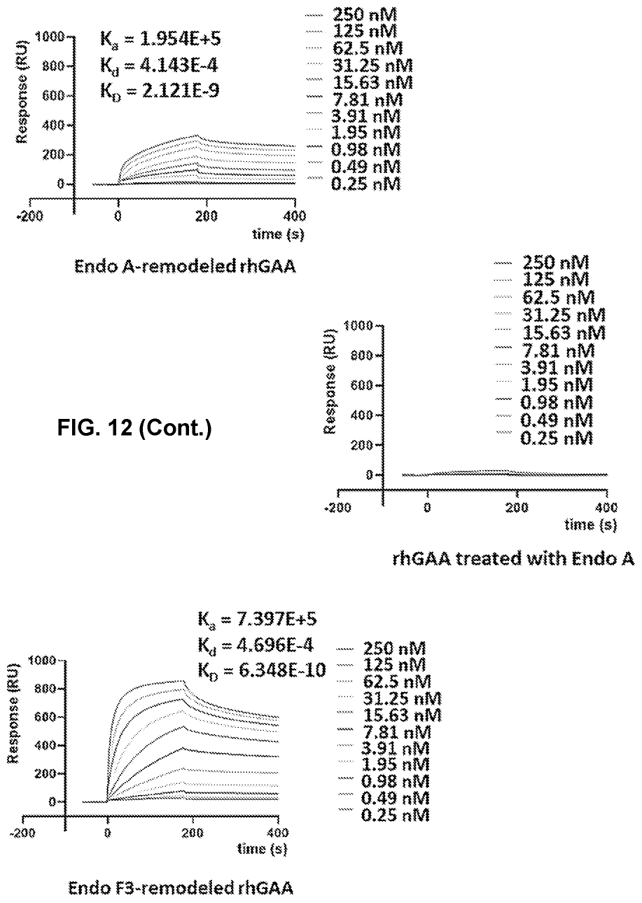

SPR experiments indicated that the native rhGAA had a notable affinity for CI-MPR (K_{D}=14.0 nM) (FIG. 2B and FIG. 12). Upon treatment with wild-type Endo-A to remove the high-mannose N-glycans including the M6P glycans, the deglycosylated rhGAA lost its binding affinity (FIG. 12). However, after Endo-A catalyzed transglycosylation with the M6P-tetrasaccharide oxazoline (8), the remodeled rhGAA (69) exhibited a 6-fold enhanced affinity for the receptor (K_{D}=2.3 nM, FIG. 2D). On the other hand, the Endo-F3-remodeled rhGAA (70) showed a 20-fold enhanced affinity (K_{D}=0.63 nM, FIG. 2F). These results suggested that introduction of additional M6P-glycans into rhGAA resulted in further enhancement of affinity for the receptor. Previous studies have also shown that a novel rhGAA (designated as ATB200) carrying a higher M6P content that binds the CI-MPR with high affinity (apparent K_{D}, ˜2-4 nM), exhibited enhanced cellular uptake and led to much improved glycogen reduction and reversal of muscle pathology in preclinical models.⁴⁶ As a result, it was expected that the Endo-A remodeled rhGAA might exhibit comparable in vivo potency as ATB200, while the Endo-F3 remodeled enzyme might demonstrate much better therapeutic efficacy than ATB200 and commercial rhGAA, due to its much higher affinity for CI-MPR.

### Example 7: Enzyme Activity of the M6P Glycan-Remodeled rhGAA

To confirm if the remodeled enzymes still maintained their catalytic activity after M6P-glycan remodeling, the α-glucosidase activity of the commercial rhGAA, the Endo-A remodeled rhGAA (69), and the Endo-F3 remodeled rhGAA (70) were assessed using 4-methylumbelliferyl-α-D-glucopyranoside (4-MUG) as the substrate.²⁴ The results indicated that the M6P glycan remodeled rhGAA maintained full enzyme activity as the parent rhGAA (FIG. 3). These data confirmed that the glycoengineering process was mild enough without denaturing the enzyme, and the resulting M6P-glycan remodeled enzyme was stable.

### Example 8: Evaluation of the Biological Effect of the Endo-A and Endo-F3 Remodeled rhGAA in an In Vitro Model of Pompe Disease

Skeletal muscle is a major tissue affected in all forms of Pompe disease, and its response to the currently available therapy with the recombinant human GAA (rhGAA; Lumizyme) is not satisfactory.⁴⁷ To evaluate the effect of the Endo-A (69) and Endo-F3 (70) remodeled rhGAA in the disease-relevant muscle cells, GAA-deficient multinucleated myotubes (KO) were used as an in vitro cell model system for Pompe disease.^{48, 49} These myotubes are formed from conditionally immortalized myoblasts derived from the GAA knockout mice; unlike myoblasts, the differentiated myotubes replicate the primary defect of the disease, namely, the enlargement of glycogen-laden lysosomes. This physiologically relevant in vitro cell model system has been shown to closely replicate the pathogenic mechanisms of muscle tissue abnormalities in Pompe disease.^{48, 49}

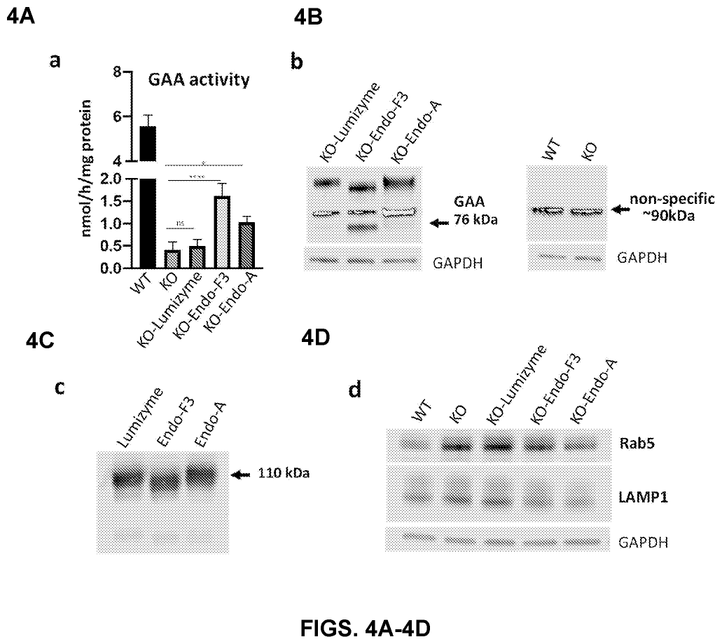

Muscle cells were exposed to the commercial rhGAA (Lumizyme), the Endo-A remodeled rhGAA (69), and Endo-F3 remodeled rhGAA (70) (5 μM for 24 hours), which reach lysosomes via mannose 6-phosphate-mediated endocytosis. KO myotubes treated with Lumizyme and the glycoengineered proteins (69 and 70) were lysed, and the GAA activity was quantified using 4-Methylumbelliferyl-α-D-glucopyranoside, a fluorogenic substrate that is routinely used for the GAA assay in the diagnosis of Pompe disease. GAA activity in the cell lysates increased significantly following incubation with both M6P-glycan remodeled proteins (69 and 70), whereas only a slight increase (statistically insignificant) was observed in Lumizyme-treated cells compared to the background level in the untreated cells (FIG. 4A). These results are consistent with the enhanced affinity of the M6P-glycan remodeled enzymes (69 and 70) to the CI-MPR receptor (FIGS. 2A-2F). The effect of the Endo-F3 remodeled rhGAA (70) was more pronounced compared to that of Endo-A remodeled protein (69), suggesting its better cellular uptake (FIG. 4A). Western blot with anti-human GAA antibodies revealed the presence of the processed, mature lysosomal form of rhGAA (76 kDa) in cells treated with the Endo-F3 remodeled protein (70) (FIG. 4B, left panel); a weaker band was also detected in cell lysates following incubation with the Endo-A remodeled enzyme (69). In contrast, the 76 kDa form was barely detectable in cells treated with Lumizyme. The available anti-human GAA antibodies detected a strong non-specific band in mouse muscle cells as indicated by its presence in both WT and GAA-deficient cells (FIG. 4B, right panel). Previous studies have shown that the mature 76 kDa form of GAA has a higher affinity and activity toward glycogen.^{31, 50}

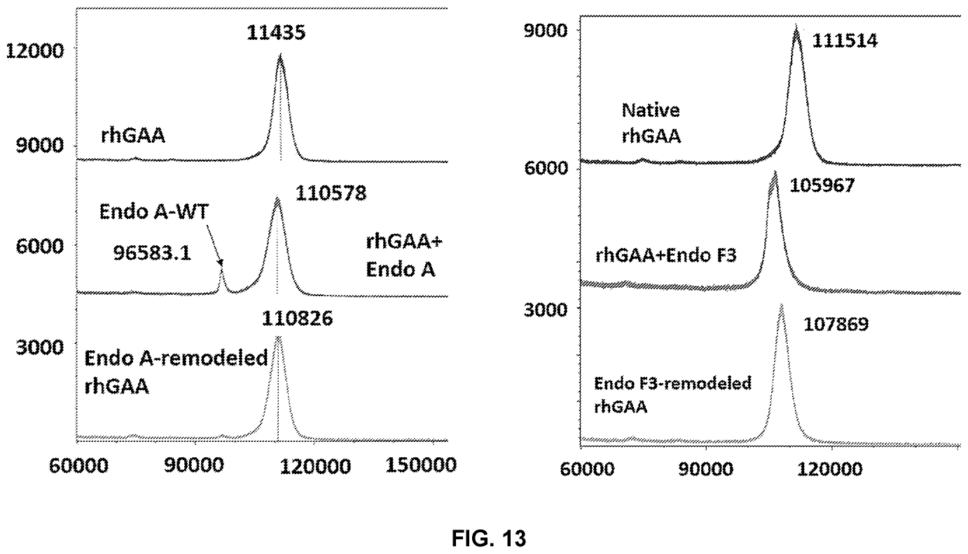

The immunoblot also showed that the molecular weight of the internalized GAA precursor appeared to be lower than the expected 110 kDa in the samples treated with the Endo-F3 remodeled rhGAA (70) compared to those treated with Lumizyme or the Endo-A remodeled rhGAA (69). The lower molecular weight of the Endo-F3 remodeled enzyme (70) was confirmed by Western analysis of the three recombinant proteins stained with anti-human GAA antibodies (FIG. 4C). These data were also consistent with the MALDI-TOF MS analysis of the glycan-remodeled rhGAA (69 and 70), which showed that the Endo-A remodeled rhGAA (69) had an average molecular mass of ca. 110 kDa, while the Endo-F3 remodeled rhGAA (70) had a molecular mass of ca. 108 kDa (FIG. 13). This observation was expected, as Endo-F3 based remodeling replaced most of the complex type N-glycans in the rhGAA with a much smaller (pentasaccharide) M6P N-glycan while the Endo-A based remodeling only changed the two high-mannose N-glycans to the M6P N-glycans. The efficient lysosomal trafficking of the remodeled proteins was associated with a significant reduction in the levels of both endosomal (Rab5) and lysosomal (LAMP1) markers, suggesting a reversal of lysosomal swelling; in contrast, the levels of these markers in Lumizyme-treated cells remained the same as in the untreated GAA-deficient cells (FIG. 4D).

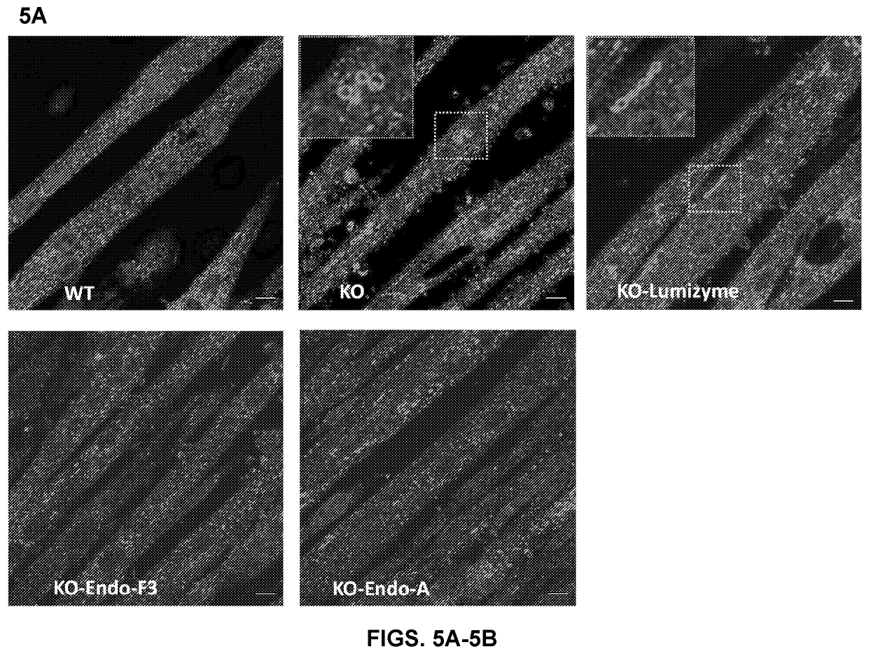

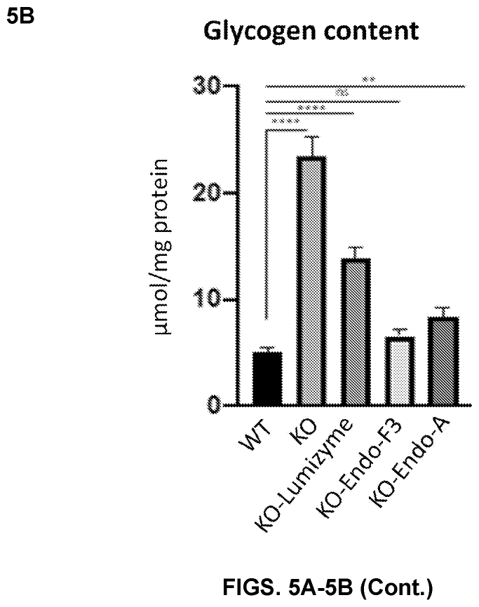

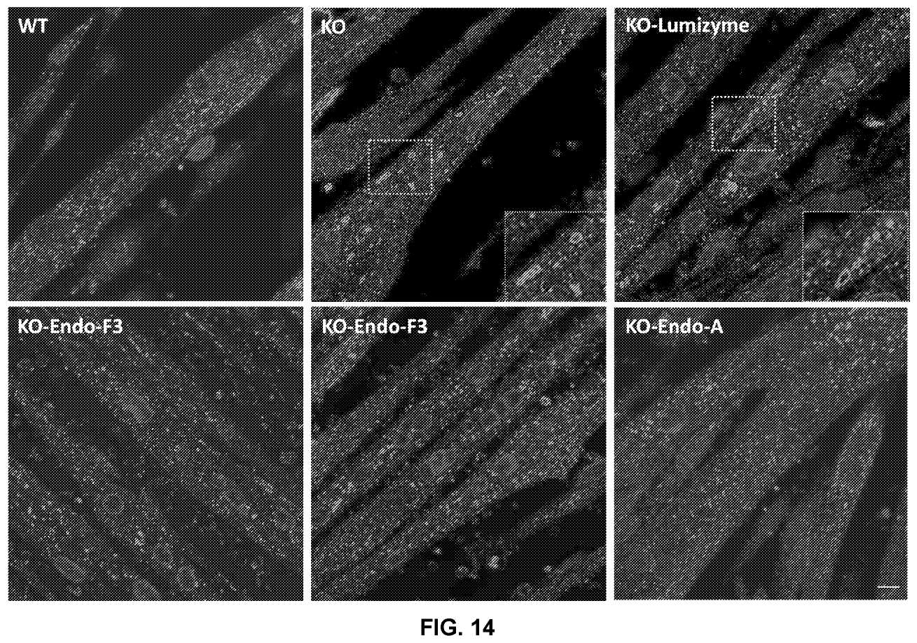

The effect of the Endo-A and Endo-F3 remodeled rhGAA was confirmed by immunostaining of myotubes with Lamp1. Enlarged lysosomes were seen in cells treated with Lumizyme but not in those treated with the remodeled enzymes (69 and 70) (FIG. 5A; see also additional images in FIG. 14). Finally, the degree of glycogen reduction achieved with Endo-A and Endo-F3 remodeled rhGAA (69 and 70) was significantly greater compared to that with the commercial rhGAA. Importantly, glycogen content in the diseased cells returned to the WT level following treatment with Endo-F3 remodeled rhGAA (70) (FIG. 5B). Taken together, these in vitro data indicated that upon efficient internalization, the M6P-glycan remodeled proteins, in particular, the Endo-F3 remodeled rhGAA, could efficiently traffic to their correct cellular destination, the lysosome, and do their job there, i.e., break down the accumulated glycogen. The therapeutic potential of the M6P glycan-remodeled rhGAA in lysosomal glycogen reduction and reversal of muscle pathology will be further evaluated in Pompe disease animal models in future studies.

In conclusion, described herein are the chemical synthesis of an array of mannose-6-phosphate (M6P) containing N-glycan oxazolines and their use as donor substrates for chemoenzymatic synthesis of M6P-containing glycopeptides and for the glycan remodeling of a therapeutic lysosomal enzyme (rhGAA). The present study revealed a Man6P-α1,2-Man disaccharide as an essential structural motif for high-affinity binding to the M6P receptor (CI-MPR). Structure-activity relationship studies identified a tetrasaccharide oxazoline carrying this M6P disaccharide motif as the minimal donor substrate for efficient transglycosylation to give high-affinity M6P ligands. The discovery on the resistance of the M6P product to the hydrolysis by wild-type Endo-A and Endo-F3, coupled with the excellent hydrolysis activity of the wild-type enzymes on high-mannose and core-fucosylated complex type N-glycans, respectively, enabled a site-selective and one-pot conjugation of the high-affinity M6P glycan ligands either at the high-mannose or complex type N-glycosylation sites in the multiply glycosylated protein, giving structurally well-defined product. The Endo-A and Endo-F3 remodeled rhGAAs maintained full enzyme activities and demonstrated 6- and 20-fold enhanced binding affinities for CI-MPR, respectively. Moreover, by using an in vitro cell model system for Pompe disease, it was demonstrated that the M6P-glycan remodeled rhGAA showed significantly enhanced cellular uptake over the commercial Lumizyme and exhibited much more efficient glycogen reduction in lysosomes than Lumizyme. While the therapeutic potential of the M6P glycan-remodeled rhGAA may be further evaluated in Pompe disease animal models, the present study provides a general and efficient method for site-selective M6P-glycan remodeling of recombinant lysosomal enzymes to achieve enhanced M6P receptor binding and cellular uptake, which holds a great promise for improved overall therapeutic efficacy of enzyme replacement therapy.

### Example 9: Chemical Synthesis

### Materials and Methods

All chemicals, reagents, and solvents were purchased from Sigma-Aldrich and TCI and unless specially noted applied in the reaction without further purification. TLC was performed using silica gel on glass plates (Sigma-Aldrich), and spots were detected under UV light (254 nm) then charring with 5% (v/v) sulfuric acid in EtOH or cerium molybdate stain (CAM) followed by heating at 150° C. Silica gel (200-425 mesh) for flash chromatography was purchased from Sigma-Aldrich. NMR spectra were recorded on a 400 MHz spectrometer (Bruker, Tokyo, Japan) with CDCl₃ or D₂O as the solvent. The chemical shifts were assigned in ppm, and multiplicities are indicated by s (singlet), d (doublet), t (triplet), q (quartet), and m (multiplet). Coupling constants (J) are reported in Hertz. MALDI-TOF was performed on a Bruker Autoflex Speed Mass Spectrometer in positive reflectron mode with DHB (ACN/H₂O=1:1) as the matrix. HRMS was performed on an Exactive Plus Orbitrap Mass Spectrometer (Thermo Scientific) equipped with a C18 column. Analytical RP-HPLC was performed on a Waters 626 HPLC instrument with a C18 column (3.5 μm, 4.6×250 mm) at 50° C. The column was eluted with a linear gradient containing 0.1% FA for 30 min at the flow rate of 1.0 mL/min. Preparative HPLC was performed with a Waters 600 HPLC instrument and Waters C18 columns (5.0 μm, 10×250 mm; 7.0 μm, 19×300 mm). The column was eluted with a suitable gradient of MeCN—H₂O containing 0.1% TFA or FA at a flow rate of 4 mL/min or 10 mL/min. DIONEX HPAEC-PAD was performed on a Thermo Scientific Dionex ICS-6000 instrument and a PA200 column using a gradient of A (100 mM NaOH) and B (100 mM NaOH and 250 mM NaOAc) at a flow rate of 0.5 mL/min (0-60% B, 30 min).

### 2-Azido-4,6-O-benzylidene-2-deoxy-αβ-D-glucopyranoside (11)

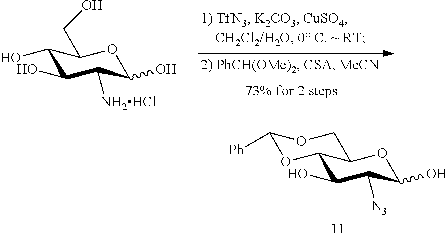

In situ preparation of TfN₃: To a vigorously stirring solution of NaN₃ (3.00 g, 46.1 mmol) in H₂O/CH₂Cl₂ (1:1, 10 mL) was dropwise added Tf₂O (1.50 mL, 8.95 mmol) at 0° C. The resulting mixture turned into white cloudy solution and was stirred at 0° C. for a further 1.5 h. The organic layer was washed with water and saturated Na₂CO₃ (aq.), and the resulting solution of TfN₃ (in about 10 mL of CH₂Cl₂) was directly used in the next step without further purification.

Cu^{II}-catalyzed diazo transfer and protection with benzylidene: The freshly prepared solution of TfN₃ (in CH₂Cl₂) was added to a solution of D-glucosamine hydrochloride (1.00 g, 4.65 mmol), K₂CO₃ (0.75 g, 5.43 mmol), and a catalytic amount of CuSO₄ (10 mg) in water (5 mL) at 0° C. Then, the ice bath was removed and MeOH was added to make the reaction homogeneous. After vigorous stirring at RT for 18 h, the mixture was passed through a pad of Celite and the filtrate was concentrated under reduced pressure to give a dry residue that was purified by a short column of silica gel (CH₂Cl₂/MeOH=5:1˜4:1) to give the crude azide product. To the crude azide product in MeCN (10 mL) was added (+)−10-Camphorsulfonic acid (195 mg, 0.84 mmol) and benzaldehyde dimethyl acetal (2.0 mL, 13.3 mmol), and the mixture was stirred at room temperature overnight. After the completion of the reaction as monitored by TLC, triethylamine was added to quench the reaction. Flash chromatography (hexanes/EtOAc=2:1) afforded the product 11 as white solid (1.00 g, 73% for 2 steps, mixture of a/ß isomers, 1.7:1). R_{f}=0.20 (hexanes/EtOAc=2:1). Spectroscopic data were in agreement with literature values.^{[1]}

### Benzyl 2-azido-3-O-benzyl-4,6-O-benzylidene-2-deoxy-β-D-glucopyranoside (12)

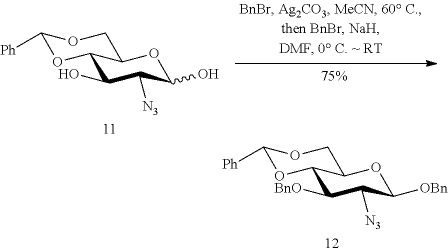

To a solution of compound 11 (952 mg, 3.25 mmol) in anhydrous acetonitrile (20 mL) was added Ag₂CO₃ (4.48 g, 16.24 mmol) and benzyl bromide (1.54 mL, 13.00 mmol), the mixture was kept in dark and heated to 60° C. overnight. When TLC showed the disappearance of the starting material, indicating the complete protection of the anomeric hydroxyl, the mixture was filtered through a pad of Celite and the filtrate was concentrated to dryness. The residue was dissolved in dry N,N-dimethylformamide (20 mL) and cooled to 0° C., sodium hydride (325 mg, 8.13 mmol) and benzyl bromide (776 μL, 6.50 mmol) were added successively, and the mixture was slowly warmed to room temperature. After the completion of the reaction as monitored by TLC, MeOH (0.5 mL) was added to quench the excess sodium hydride. The reaction was diluted with CH₂Cl₂, successively washed with H₂O and brine and dried over anhydrous Na₂SO₄. Flash column chromatography (hexanes/EtOAc=10:1˜8:1) gave 12 (1.151 g, 75%) as white solid. R_{f}=0.30 (hexanes/EtOAc=10:1); ¹H NMR (400 MHZ, CDCl₃) δ 7.54-7.52, 7.43-7.30 (15H, m, Ar—H), 5.63 (1H, s, PhCH), 4.97 (1H, d, PhCH₂, J=11.8 Hz), 4.96 (1H, d, PhCH₂, J=11.2 Hz), 4.84 (1H, d, PhCH₂, J=11.2 Hz), 4.73 (1H, d, PhCH₂, J=11.8 Hz), 4.48 (1H, d, H−1, J=7.8 Hz), 4.42 (1H, dd, J=10.5 Hz, J=5.0 Hz), 3.86 (1H, dd, J=10.3 Hz, J=10.3 Hz), 3.77 (1H, dd, J=9.2 Hz, J=9.2 Hz), 3.61-3.56 (2H, m), 3.43 (1H, m); ¹³C NMR (100 MHZ, CDCl₃) δ 137.87, 137.17, 136.51, 129.10, 128.57, 128.41, 128.33, 128.23, 128.17, 128.07, 127.91, 126.04, 101.37, 101.14, 81.61, 79.06, 74.96, 71.43, 68.63, 66.25; MALDI-TOF: [M+Na]⁺ calcd for C₂₇H₂₇N₃NaO₅⁺, 496.18; found, 495.91.

### Benzyl 2-azido-3,6-di-O-benzyl-2-deoxy-β-D-glucopyranoside (13)

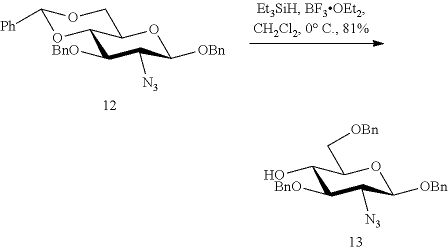

To a solution of compound 12 (500 mg, 1.056 mmol) in anhydrous CH₂Cl₂ (12 mL) was added triethylsilane (1.01 mL, 6.34 mmol) and BF₃·OEt₂ (0.67 mL, 5.28 mmol) at 0° C., the mixture was stirred at this temperature for 3 h and quenched by triethylamine. The residue was concentrated and purified by flash column chromatography (hexanes/EtOAc=10:1˜4:1) to give 13 (406 mg, 81%) as colorless syrup. R_{f}=0.30 (hexanes/EtOAc=4:1); ¹H NMR (400 MHZ, CDCl₃) δ 7.44-7.35 (15H, m, Ar—H), 4.98-4.93 (2H, m, PhCH₂), 4.81 (1H, d, PhCH₂, J=11.3 Hz), 4.72 (1H, d, PhCH₂, J=11.9 Hz), 4.68-4.59 (2H, m, PhCH₂), 4.40 (1H, d, H−1, J=8.1 Hz), 3.79 (2H, m), 3.69 (1H, m), 3.52-3.42 (2H, m), 3.28 (1H, dd, J=9.3 Hz, J=9.3 Hz), 2.70 (1H, m); ¹³C NMR (100 MHz, CDCl₃) δ 138.13, 137.76, 136.77, 128.64, 128.53, 128.51, 128.15, 128.07, 128.05, 128.02, 127.90, 127.77, 100.64, 82.62, 75.13, 74.06, 73.77, 71.96, 71.02, 70.17, 65.79; MALDI-TOF: [M+Na]⁺ calcd for C₂₇H₂₉N₃NaO₅, 498.20; found, 498.18.

### Benzyl 2-O-benzyl-4,6-O-benzylidene-3-O-p-methoxybenzyl-β-D-mannopyranosyl-(1→4)-2-azido-3,6-di-O-benzyl-2-deoxy-β-D-glucopyranoside (15)

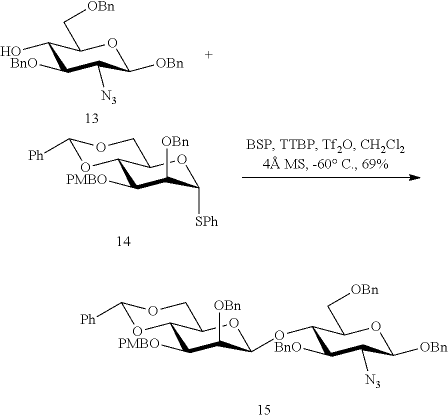

To a solution of compound 14^{[2]} (581 mg, 1.019 mmol) in anhydrous CH₂Cl₂ (18 mL) was added activated 4 Å molecular sieves (2.0 g) under argon atmosphere, the mixture was stirred for 2 h at room temperature, then cooled to −60° C. BSP (236 mg, 1.13 mmol) and TTBP (427 mg, 2.05 mmol) were added, and the solution was kept at −60° C. for 40 min before Tf₂O (206 μL, 1.23 mmol) was added. After 20 min, a solution of compound 13 (323 mg, 0.68 mmol) in CH₂Cl₂ (3.0 mL) was added and the mixture was stirred at −60° C. for 3 h. After the completion of the reaction as monitored by TLC, the mixture was filtered through a Celite pad. The filtrate was poured into saturated NaHCO₃ and extracted with CH₂Cl₂. The organic layer was washed with brine, dried over Na₂SO₄, and concentrated. The residue was purified by column chromatography (hexanes/EtOAc=10:1˜5:1) to afford 15 (441 mg, 69%) as a colorless syrup. R_{f}=0.40 (hexanes/EtOAc=4:1); Spectroscopic data were in agreement with literature values. (31 MALDI-TOF: [M+Na]⁺ calcd for C₅₅H₅₇N₃NaO₁₁⁺, 958.39; found, 958.53.

### Benzyl 2,4-di-O-benzyl-3-O-p-methoxybenzyl-β-D-mannopyranosyl-(1→4)-2-azido-3,6-di-O-benzyl-2-deoxy-β-D-glucopyranoside (16)

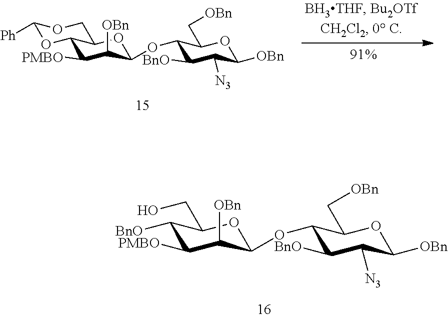

To a solution of compound 15 (300 mg, 0.321 mmol) in BH₃·THF (4.0 mL) was added a solution of Bu₂BOTf in CH₂Cl₂ (1 M, 642 μL) under argon atmosphere at 0° C. and the mixture was stirred at 0° C. for 40 min when TLC indicated the completion of the reaction. Et₃N (300 μL) was added to the reaction followed by careful addition of MeOH (600 μL). The mixture was co-evaporated with MeOH three times and the residue was purified by flash chromatography (hexanes/EtOAc=5:1˜2:1) to afford 16 (274 mg, 91%) as a colorless syrup. R_{f}=0.30 (hexanes/EtOAc=3:1); ¹H NMR (400 MHZ, CDCl₃) δ 7.50-7.28 (27H, m, Ar—H), 6.93 (2H, m, Ar—H), 5.16 (1H, d, PhCH₂, J=10.8 Hz), 5.01 (1H, d, PhCH₂, J=12.1 Hz), 4.97-4.86 (3H, m, PhCH₂), 4.78 (1H, d, PhCH₂, J=12.1 Hz), 4.75-4.64 (3H, m, PhCH₂), 4.55-4.52 (3H, m, PhCH₂), 4.40 (1H, d, J=8.1 Hz), 4.01 (1H, dd, J=9.3 Hz, J=9.3 Hz), 3.89-3.75 (6H, m), 3.73-3.67 (2H, m), 3.57 (1H, dd, J=8.2 Hz, J=8.2 Hz), 3.45-3.39 (4H, m), 3.22-3.18 (1H, m), 1.97 (1H, s); ¹³C NMR (100 MHz, CDCl₃) δ 159.31, 138.76, 138.52, 138.47, 137.75, 136.93, 130.38, 129.20, 128.64, 128.56, 128.45, 128.38, 128.26, 128.17, 128.06, 128.03, 127.91, 127.78, 127.69, 127.65, 127.51, 113.89, 100.76, 100.51, 82.34, 81.50, 77.08, 75.80, 75.31, 75.10, 74.90, 74.83, 74.57, 73.71, 71.65, 70.93, 68.54, 65.96, 62.22, 55.34; MALDI-TOF: [M+Na]⁺ calcd for C₅₅H₅₉N₃NaO₁₁⁺, 960.40; found, 959.98.

### Benzyl 2,4-di-O-benzyl-3-O-p-methoxybenzyl-β-D-mannopyranosyl-(1→4)-2-acetamido-3,6-di-O-benzyl-2-deoxy-β-D-glucopyranoside (17)

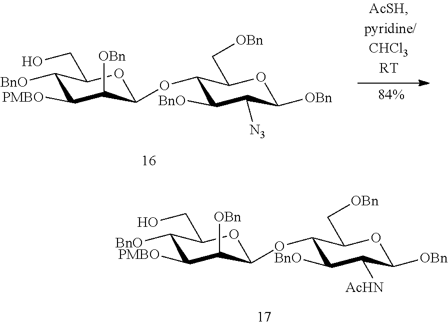

A solution of compound 16 (133.5 mg, 0.142 mmol) in a mixture of AcSH/pyridine/CHCl₃ (0.8 mL/0.6 mL/0.8 mL) was stirred at room temperature for 18 h. After the completion of the reaction as monitored by TLC, the resulting mixture was concentrated and subjected to flash chromatography on silica gel (hexanes/EtOAc=4:1˜3:2) to afford compound 17 (113.8 mg, 84%) as colorless syrup. R_{f}=0.30 (hexanes/EtOAc=2:1); ¹H NMR (400 MHZ, CDCl₃) δ 7.44-7.43, 7.37-7.23 (27H, m, Ar—H), 6.88-6.86 (2H, m, Ar—H), 5.75 (1H, d, NH, J=8.0 Hz), 4.98-4.81 (6H, m, PhCH₂), 4.65-4.59 (4H, m, PhCH₂), 4.53-4.47 (4H, m, PhCH₂), 4.16 (1H, dd, J=7.8 Hz, J=7.8 Hz), 3.90 (1H, dd, J=7.0 Hz, J=7.0 Hz), 3.86-3.78 (6H, m), 3.73-3.69 (3H, m), 3.64-3.58 (1H, m), 3.53-3.48 (1H, m), 3.42-3.39 (1H, m), 3.22-3.18 (1H, m), 2.09 (1H, s), 1.76 (3H, s); ¹³C NMR (100 MHz, CDCl₃) δ 170.54, 159.25, 138.78, 138.63, 138.37, 137.91, 137.60, 130.28, 129.17, 128.51, 128.39, 128.33, 128.26, 128.15, 128.02, 127.88, 127.74, 127.66, 127.61, 127.54, 113.84, 101.08, 99.14, 82.18, 77.78, 75.71, 75.47, 75.13, 75.08, 74.76, 74.62, 73.75, 73.57, 71.59, 70.71, 69.38, 62.28, 55.29, 23.38; MALDI-TOF: [M+Na]⁺ calcd for C₅₇H₆₃NNaO₁₂⁺, 976.42; found, 976.00.

### Benzyl 2,4-di-O-benzyl-6-O-dibenzylphosphonato-3-O-p-methoxybenzyl-β-D-mannopyranosyl-(1→4)-2-acetamido-3,6-di-O-benzyl-2-deoxy-β-D-glucopyranoside (18)

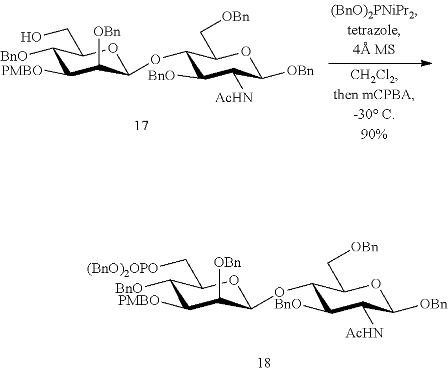

To a solution of compound 17 (50.0 mg, 0.052 mmol) in anhydrous CH₂Cl₂ (2.0 mL) was added activated 4 Å molecular sieves (200 mg) and tetrazole (0.45 M in MeCN, 582 μL) and the mixture was stirred at room temperature for 1.5 h before (BnO)₂PNiPr₂ (70.6 μL) was added. The resulting mixture was further stirred overnight under argon atmosphere at room temperature until the complete disappearance of the starting material. Then the reaction was cooled to −30° C., and mCPBA (77 wt %, 61.5 mg) was added, the reaction mixture was stirred at this temperature for 1 h and then filtered through a Celite pad. The filtrate was diluted with CH₂Cl₂, washed with saturated NaHCO₃(aq.), dried over Na₂SO₄ and concentrated to dryness. The residue was purified by flash chromatography to give compound 18 (56.8 mg, 90%) as colorless syrup. R_{f}=0.30 (hexanes/Acetone=2:1); ¹H NMR (400 MHZ, CDCl₃) δ 7.42-7.40, 7.34-7.20 (37H, m, Ar—H), 6.87-6.85 (2H, m, Ar—H), 5.96 (1H, d, NH, J=8.3 Hz), 4.99-4.92 (4H, m, PhCH₂), 4.89 (1H, d, J=11.9 Hz, PhCH₂), 4.88 (1H, d, J=10.8 Hz, PhCH₂), 4.85-4.82 (3H, m, PhCH₂), 4.79 (1H, d, J=11.8 Hz, PhCH₂), 4.65 (1H, d, J=11.8 Hz, PhCH₂), 4.60-4.55 (3H, m), 4.52 (1H, d, J=11.7 Hz, PhCH₂), 4.47-4.41 (2H, m), 4.25-4.20 (1H, m), 4.19-4.12 (1H, m), 4.06 (1H, dd, J=7.1 Hz, J=7.1 Hz), 3.97 (1H, dd, J=6.9 Hz, J=6.9 Hz), 3.85-3.79 (6H, m), 3.77-3.71 (2H, m), 3.40 (1H, dd, J=2.8 Hz, J=9.3 Hz), 3.35-3.32 (1H, m), 1.69 (3H, s); ¹³C NMR (100 MHz, CDCl₃) δ 170.07, 159.30, 138.87, 138.51, 138.17, 138.08, 137.80, 135.85, 135.78, 130.05, 129.24, 128.44, 128.39, 128.29, 128.24, 128.21, 128.11, 128.05, 128.03, 127.95, 127.93, 127.77, 127.73, 127.69, 127.58, 127.54, 127.38, 113.85, 100.92, 99.61, 81.98, 77.19, 77.13, 75.19, 75.11, 74.93, 74.54, 74.38, 74.30, 73.86, 73.49, 72.73, 71.49, 70.45, 69.68, 69.34, 69.29, 66.68, 66.63, 55.28, 53.58, 23.17; 31P NMR (146 MHz, CDCl₃) δ −1.20; MALDI-TOF: [M+Na]⁺ calcd for C₇₁H₇₆NNaO₁₅P⁺, 1236.48; found, 1236.25.

### 6-O-phosphonato-β-D-mannopyranosyl-(1→4)-2-acetamido-2-deoxy-αβ-D-glucopyranoside (19)

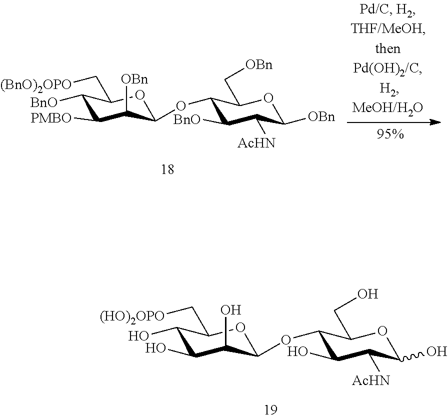

A mixture of compound 18 (56.8 mg, 0.047 mmol) and Pd/C (10 wt. % loading, 30 mg) in MeOH (2.0 mL) and THF (2.0 mL) was stirred under H₂ atmosphere for 21 h. The reaction mixture was filtered through a Celite pad, then concentrated to dryness. The mixture of the residue and Pd(OH)₂/C (20 wt. % loading, 30 mg) in MeOH (2.5 mL) and H₂O (2.5 mL) was stirred under H₂ atmosphere for further 21 h. The reaction mixture was filtered through a Celite pad. The filtrate was concentrated to dryness then dissolved in H₂O and lyophilized. The crude product was purified on a Sephadex G-10 column by elution with H₂O. Fractions containing the product were pooled and lyophilized to give compound 19 (20.5 mg, 95%) as white solid. R_{f}=0.50 (n-BuOH/EtOH/H₂O/AcOH=1:1:1:0.05); ¹H NMR (400 MHZ, D₂O) δ 5.15 (0.54H, d, J=3.4 Hz), 4.65 (0.44H, d, J=8.2 Hz), 4.05-3.97 (2.15H, m), 3.96-3.92 (1.07H, m), 3.90-3.86 (1.28H, m), 3.83-3.78 (1.12H, m), 3.77-3.74 (0.76H, m), 3.73-3.71 (0.53H, m), 3.71-3.68 (0.77H, m), 3.68-3.63 (2.64H, m), 3.62-3.59 (1.06H, m), 3.54-3.51 (0.44H, m), 3.45-3.41 (0.99H, m), 1.97 (3H, s); ¹³C NMR (100 MHZ, D₂O) δ 174.36, 174.06, 100.01, 99.86, 94.55, 90.04, 79.36, 78.89, 75.10, 75.05, 74.21, 72.10, 71.76, 70.20, 70.17, 69.63, 68.64, 65.72, 63.06, 59.97, 59.83, 55.80, 53.35, 21.81, 21.50; 31P NMR (146 MHZ, D₂O) δ 2.76 (overlapped signals); HRMS: [M+H]⁺ calcd for C₁₄H₂₇NO14P⁺, 464.1164; found, 464.1169.

### 2-Methyl-[6-O-phosphonato-β-D-mannopyranosyl-(1→4)-1,2-dideoxy-α-D-glucopyrano]-[2,1-d]-2-oxazoline (5)

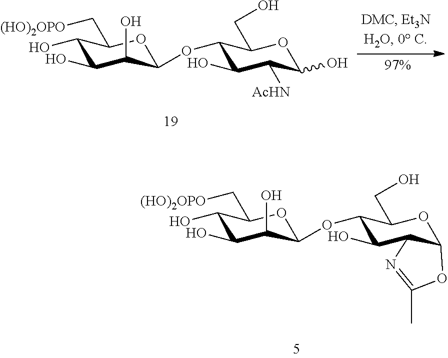

To a solution of compound 19 (5.0 mg, 0.011 mmol) in H₂O (300 μL) were added Et₃N (60.5 μL) and 2-chloro-1,3-dimethylimidazolinium chloride (DMC, 36.6 mg) at 0° C. The reaction mixture was monitored by DIONEX HPAEC-PAD. After 2 h, the HPAEC analysis indicated that the free oligosaccharide was converted into a new oligosaccharide that was eluted earlier than the reducing sugar under the HPAEC condition (see general method). The product was purified by gel filtration on a Sephadex G-10 column that was eluted with 0.1% aq Et₃N to afford compound 5 (4.7 mg, 97%) as white solid after lyophilization with 5 mol. % of NaOH. 1H NMR (400 MHZ, D₂O) δ 6.01 (1H, d, J=7.3 Hz), 4.63 (1H, m), 4.36-4.47 (1H, m), 4.13-4.11 (1H, m), 3.99-3.89 (3H, m), 3.72-3.59 (3H, m), 3.58-3.54 (2H, m), 3.38-3.32 (2H, m), 1.99 (3H, d, J=1.7 Hz); ¹³C NMR (100 MHz, D₂O) δ 168.70, 101.34, 99.88, 77.48, 75.88, 75.81, 72.57, 71.02, 70.50, 68.99, 66.54, 65.36, 63.25, 63.21, 61.82, 12.96; 31p NMR (146 MHZ, D₂O) δ 4.44. HRMS: [M+H]⁺ calcd for C₁₄H₂₅NO13P⁺, 446.1058; found, 446.1064.

### Benzyl 2-O-benzyl-4,6-O-benzylidene-β-D-mannopyranosyl-(1→4)-2-azido-3,6-di-O-benzyl-2-deoxy-β-D-glucopyranoside (20)

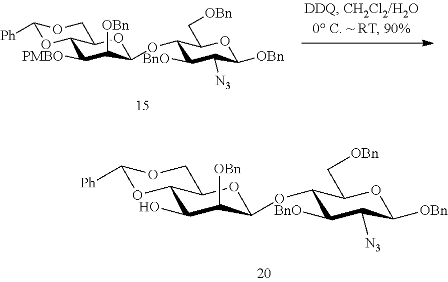

To a solution of 15 (454 mg, 0.485 mmol) in a mixture of CH₂Cl₂/H₂O (15 mL/1 mL) was added DDQ (252 mg, 1.11 mmol) at 0° C. After 30 min, the reaction mixture was warmed to room temperature and further stirred for 1 h. The reaction mixture was diluted with CH₂Cl₂, washed with saturated NaHCO₃(aq.) and brine, and dried over Na₂SO₄. Concentration and purification by column chromatography on silica gel (hexanes/EtOAc=6:1˜3:1) provided 20 (356 mg, 90%) as a white amorphous solid. R_{f}=0.25 (hexanes/EtOAc=3:1); Spectroscopic data were in agreement with literature values.^{[3]} MALDI-TOF: [M+Na]⁺ calcd for C₄₇H₄₉N₃NaO₁₀, 838.33; found, 838.47.

### Benzyl 3,4-di-O-benzyl-2-O-benzoyl-6-O-triisopropylsilyl-α-D-mannopyranosyl-(1→6)-2,4-di-O-benzyl-3-0)-p-methoxybenzyl-β-D-mannopyranosyl-(1→4)-2-azido-3,6-di-O-benzyl-2-deoxy-β-D-glucopyranoside (23)

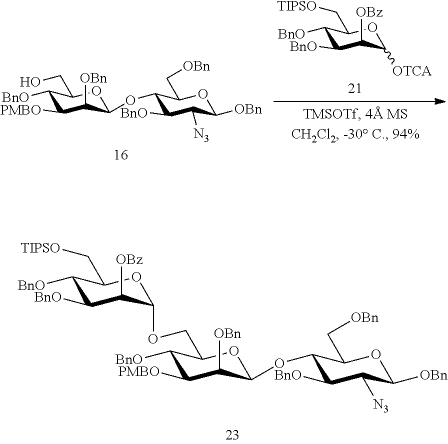

A mixture of trichloroacetimidate donor 21^{[4]} (100 mg, 0.13 mmol), acceptor 16 (63 mg, 0.067 mmol) and activated 4 Å molecular sieves (200 mg) in anhydrous CH₂Cl₂ (2.0 mL) was stirred at room temperature under argon atmosphere for 1.5 h, and then cooled to −30° C. TMSOTf (1.0 μL, 5.5 μmol) was added. After stirring at −30° C. for 50 min, the mixture was quenched with triethylamine (20 μL) and filtered then concentrated in vacuo. The residue was purified via silica gel chromatography (hexanes/EtOAc=10:1˜4:1) to give product 23 (97.6 mg, 94%) as white foam. R_{f}=0.50 (hexanes/EtOAc=3:1); ¹H NMR (400 MHZ, CDCl₃) δ 8.10-8.08, 7.58, 7.45-7.13 (42H, m, Ar—H), 6.88-6.86 (2H, m, Ar—H), 5.66-5.64 (1H, m), 5.10 (1H, d, J=11.4 Hz, PhCH₂), 4.95-4.77 (7H, m, PhCH₂), 4.70-4.57 (5H, m), 4.52-4.39 (4H, m), 4.34-4.31 (2H, m), 4.08 (1H, dd, J=9.5 Hz, J=9.5 Hz), 4.02-3.97 (2H, m), 3.91-3.86 (2H, m), 3.82-3.76 (5H, m), 3.72-3.68 (3H, m), 3.63-3.59 (2H, m), 3.52 (1H, dd, J=8.3 Hz, J=9.7 Hz), 3.43-3.33 (4H, m), 1.09 (21H, s); ¹³C NMR (100 MHz, CDCl₃) δ 165.56, 159.28, 139.08, 138.90, 138.61, 138.51, 138.15, 138.01, 137.00, 132.97, 130.24, 130.16, 130.05, 129.29, 128.56, 128.46, 128.31, 128.21, 128.18, 128.14, 128.01, 127.93, 127.85, 127.78, 127.67, 127.61, 127.52, 127.43, 127.41, 127.36, 127.26, 113.85, 101.38, 100.73, 97.98, 82.63, 80.94, 78.23, 77.30, 75.04, 74.91, 74.85, 74.49, 74.44, 74.07, 73.87, 73.58, 72.75, 71.46, 71.31, 70.93, 69.05, 68.74, 66.73, 65.97, 62.30, 55.30, 18.10, 18.07, 12.06; MALDI-TOF: [M+Na]⁺ calcd for C₉₁H₁₀₅N₃NaO₁₇Si⁺, 1562.71; found, 1563.13.

### Benzyl 3,4-di-O-benzyl-2-O-benzoyl-6-O-triisopropylsilyl-α-D-mannopyranosyl-(1→3)-2-O-benzyl-4,6-O-benzylidene-β-D-mannopyranosyl-(1→4)-2-azido-3,6-di-O-benzyl-2-deoxy-β-D-glucopyranoside (24)

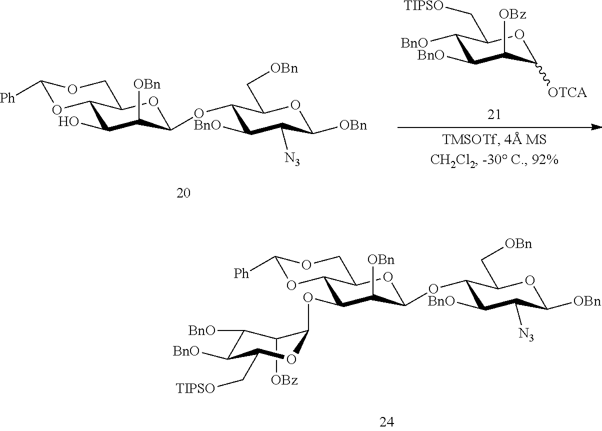

A mixture of trichloroacetimidate donor 21^{[4]} (187 mg, 0.245 mmol), acceptor 20 (100 mg, 0.123 mmol) and activated 4 Å molecular sieves (300 mg) in anhydrous CH₂Cl₂ (3.0 mL) was stirred at room temperature under argon atmosphere for 1.5 h, and then cooled to −30° C. TMSOTf (2.1 μL, 12.3 μmol) was added. After stirring at −30° C. for 1 h, the mixture was quenched with triethylamine (20 μL) and filtered then concentrated in vacuo. The residue was purified via silica gel chromatography (hexanes/EtOAc=15:1˜5:1) to give product 23 (159.4 mg, 92%) as white foam. R_{f}=0.30 (hexanes/EtOAc=8:1); ¹H NMR (400 MHZ, CDCl₃) δ 8.01-7.98, 7.49, 7.36-7.09 (40H, m, Ar—H), 5.74 (1H, m), 5.45 (1H, s), 5.30 (1H, m), 4.96 (1H, m), 4.85-4.82 (2H, m), 4.71-4.48 (8H, m), 4.38-4.35 (2H, m), 4.22-4.20 (1H, m), 4.04-3.88 (6H, m), 3.83-3.80 (1H, m), 3.72-3.70 (1H, m), 3.68-3.63 (2H, m), 3.60-3.57 (1H, m), 3.51-3.48 (1H, m), 3.42-3.35 (2H, m), 3.28-3.21 (2H, m), 2.99-2.96 (1H, m), 1.00 (21H, s); ¹³C NMR (100 MHz, CDCl₃) δ 165.53, 138.85, 138.55, 138.27, 138.07, 137.66, 137.22, 136.87, 133.11, 130.00, 128.61, 128.51, 128.29, 128.17, 128.11, 128.05, 128.00, 127.97, 127.93, 127.84, 127.80, 127.72, 127.55, 125.90, 101.05, 101.01, 100.51, 98.76, 81.61, 78.79, 78.52, 78.16, 76.95, 75.66, 75.55, 75.23, 75.06, 74.98, 73.89, 73.69, 71.48, 70.90, 68.75, 68.38, 68.32, 67.03, 65.82, 62.76, 18.11, 12.07; MALDI-TOF: [M+Na]⁺ calcd for C₉₁H₁₀₅N₃NaO₁₇Si⁺, 1440.64; found, 1440.07.

### Benzyl 2,3,4-tri-O-benzyl-6-O-triisopropylsilyl-α-D-mannopyranosyl-(1→6)-2,4-di-O-benzyl-3-O-p-methoxybenzyl-β-D-mannopyranosyl-(1→4)-2-azido-3,6-di-O-benzyl-2-deoxy-β-D-glucopyranoside (25)

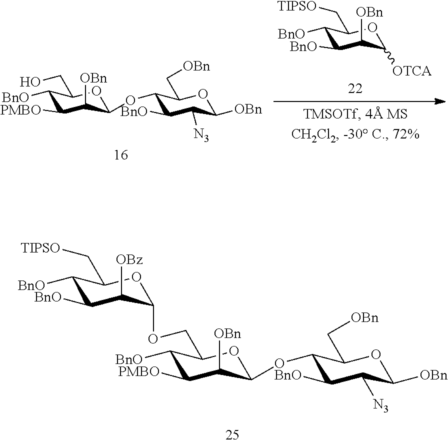

A mixture of trichloroacetimidate donor 22^{[5]} (100 mg, 0.133 mmol), acceptor 16 (63 mg, 0.067 mmol) and activated 4 Å molecular sieves (200 mg) in anhydrous CH₂Cl₂ (2.0 mL) was stirred at room temperature under argon atmosphere for 1.5 h, and then cooled to −30° C. TMSOTf (1.0 μL, 5.5 μmol) was added. After stirring at −30° C. for 50 min, the mixture was quenched with triethylamine (20 μL) and filtered then concentrated in vacuo. The residue was purified via silica gel chromatography (hexanes/EtOAc=10:1˜4:1) to give the desired α-isomer 25 (74.3 mg, 72%) as white foam. R_{f}=0.40 (hexanes/EtOAc=4:1); ¹H NMR (400 MHz, CDCl₃) δ 7.38-7.19 (42H, m, Ar—H), 6.87-6.85 (2H, m, Ar—H), 5.00 (1H, d, J=11.7 Hz, PhCH₂), 4.96-4.79 (7H, m, PhCH₂), 4.69 (1H, d, J=12.0 Hz, PhCH₂), 4.65-4.55 (4H, m), 4.50-4.40 (6H, m), 4.29 (1H, d, J=8.0 Hz), 4.01 (1H, dd, J=9.5 Hz, J=9.5 Hz), 3.97-3.89 (2H, m), 3.87-3.74 (9H, m), 3.72-3.62 (2H, m), 3.58-3.53 (2H, m), 3.46 (1H, dd, J=8.1 Hz, J=9.7 Hz), 3.39 (1H, dd, J=2.7 Hz, J=9.4 Hz), 3.37-3.32 (2H, m), 3.27-3.24 (1H, m), 1.06 (21H, s); ¹³C NMR (100 MHz, CDCl₃) δ 159.25, 139.17, 138.97, 138.92, 138.59, 138.56, 138.52, 137.99, 136.92, 130.28, 129.20, 128.51, 128.45, 128.28, 128.23, 128.22, 128.16, 128.14, 128.06, 127.92, 127.80, 127.77, 127.66, 127.61, 127.50, 127.46, 127.42, 127.32, 127.23, 127.11, 113.82, 101.61, 100.61, 98.19, 82.57, 80.75, 79.66, 77.60, 77.24, 75.42, 75.24, 74.92, 74.86, 74.75, 74.45, 74.28, 73.56, 73.41, 72.40, 71.52, 71.47, 70.90, 68.70, 66.12, 62.80, 55.28, 29.70, 18.05, 18.01, 12.04; MALDI-TOF: [M+Na]⁺ calcd for C₉₁H₁₀₇N₃NaO₁₆Si, 1549.94; found, 1549.44.

### Benzyl 2,3,4-tri-O-benzyl-6-O-triisopropylsilyl-α-D-mannopyranosyl-(1→3)-2-O-benzyl-4,6-O-benzylidene-β-D-mannopyranosyl-(1→4)-2-azido-3,6-di-O-benzyl-2-deoxy-β-D-glucopyranoside (26)

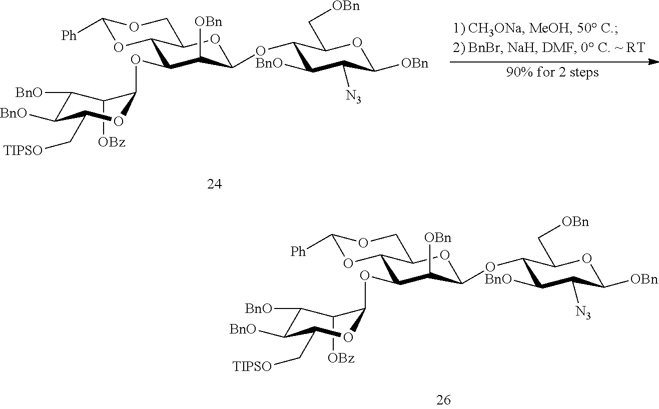

To a solution of compound 24 (90 mg, 0.0635 mmol) in MeOH (4.0 mL) was added sodium methoxide until pH=10, the solution was heated to 50° C. and stirred overnight. After the complete disappearance of the starting material, the solution was concentrated to dryness and dissolved in dry N,N-dimethylformamide (3.0 mL) and cooled to 0° C., sodium hydride (10.2 mg) and benzyl bromide (22.7 μL) were added successively, and the mixture was slowly warmed to room temperature. After the completion of the reaction as monitored by TLC, MeOH was added to quench the excess sodium hydride. The reaction was diluted with CH₂Cl₂, successively washed with H₂O and brine and dried over anhydrous Na₂SO₄. The residue was purified by flash column chromatography (hexanes/EtOAc=10:1˜6:1) to afford compound 26 (80.0 mg, 90% for 2 steps) as colorless syrup. R_{f}=0.20 (hexanes/EtOAc=6:1); 1H NMR (400 MHZ, CDCl₃) δ 7.48-7.46, 7.43-7.15, 7.10-7.08 (40H, m, Ar—H), 5.48 (1H, s), 5.34 (1H, m), 5.08 (1H, d, J=10.5 Hz, PhCH₂), 4.99-4.94 (2H, m), 4.78-4.61 (7H, m), 4.54-4.48 (3H, m), 4.43 (1H, d, J=12.4 Hz), 4.35-4.31 (2H, m), 4.07-3.98 (3H, m), 3.97-3.88 (4H, m), 3.85-3.82 (2H, m), 3.79-3.78 (1H, m), 3.73-3.68 (2H, m), 3.64-3.61 (1H, m), 3.53-3.46 (2H, m), 3.40-3.32 (2H, m), 3.10-3.03 (1H, m), 1.08-1.07 (21H, s); ¹³C NMR (100 MHZ, CDCl₃) δ 138.92, 138.58, 138.56, 138.37, 138.34, 137.76, 137.49, 136.85, 129.22, 128.57, 128.48, 128.29, 128.27, 128.21, 128.13, 128.02, 127.97, 127.94, 127.92, 127.79, 127.72, 127.60, 127.47, 127.44, 127.38, 127.20, 126.16, 101.75, 100.90, 100.46, 98.47, 81.64, 79.72, 79.07, 78.87, 77.22, 76.58, 75.69, 75.63, 75.03, 74.97, 74.81, 74.61, 74.38, 73.60, 71.82, 70.86, 68.49, 68.33, 66.89, 65.78, 63.46, 29.71, 18.09, 18.07, 12.05; MALDI-TOF: [M+Na]⁺ calcd for C₈₃H₉₇N₃NaO₁₅Si, 1426.66; found, 1426.03.

### Benzyl 2,3,4-tri-O-benzyl-6-O-triisopropylsilyl-α-D-mannopyranosyl-(1→6)-2,4-di-O-benzyl-3-O-p-methoxybenzyl-β-D-mannopyranosyl-(1→4)-2-acetamido-3,6-di-O-benzyl-2-deoxy-β-D-glucopyranoside (27)

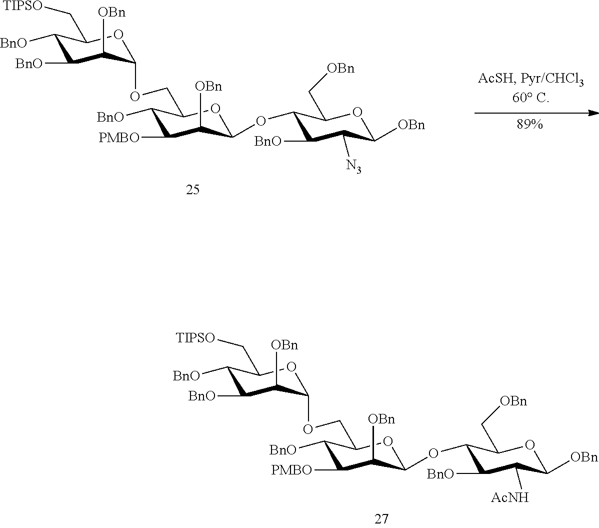

A solution of compound 25 (65.0 mg, 0.042 mmol) in a mixture of AcSH/pyridine/CHCl₃ (0.6 mL/0.4 mL/0.6 mL) was stirred at 60° ° C. for 18 h. After the completion of the reaction as monitored by TLC, the resulting mixture was concentrated and subjected to flash chromatography on silica gel (hexanes/EtOAc=8:1˜3:1) to afford compound 27 (58.1 mg, 89%) as colorless syrup. R_{f}=0.30 (hexanes/EtOAc=3:1); ¹H NMR (400 MHZ, CDCl₃) δ 7.41-7.18 (42H, m, Ar—H), 6.86-6.84 (2H, m, Ar—H), 5.33 (1H, d, J=7.9 Hz, NH), 4.95-4.83 (8H, m, PhCH/2), 4.63-4.53 (7H, m), 4.50-4.41 (6H, m), 4.06 (1H, dd, J=9.5 Hz, J=9.5 Hz), 4.03-3.97 (2H, m), 3.88-3.73 (11H, m), 3.66-3.63 (1H, m), 3.58-3.52 (4H, m), 3.44 (1H, dd, J=2.8 Hz, J=9.3 Hz), 3.33-3.29 (1H, m), 1.51 (3H, s), 1.04 (21H, s); ¹³C NMR (100 MHZ, CDCl₃) δ 169.48, 158.75, 138.81, 138.64, 138.44, 138.19, 138.00, 137.63, 137.39, 129.76, 128.69, 127.94, 127.78, 127.75, 127.71, 127.67, 127.58, 127.55, 127.47, 127.28, 127.19, 127.16, 127.07, 127.02, 126.95, 126.80, 126.77, 126.59, 113.31, 100.63, 98.84, 97.51, 82.08, 79.65, 75.19, 74.80, 74.60, 74.54, 74.45, 74.36, 74.29, 74.04, 73.98, 73.11, 73.02, 72.72, 71.91, 71.32, 70.99, 70.23, 68.98, 65.87, 62.09, 54.77, 29.19, 22.69, 17.53, 17.49, 11.56; MALDI-TOF: [M+Na]⁺ calcd for C₉₃H₁₁₁NNaO₁₇Si, 1565.98; found, 1565.46.

### Benzyl 2,3,4-tri-O-benzyl-6-O-triisopropylsilyl-α-D-mannopyranosyl-(1→3)-2-O-benzyl-4,6-O-benzylidene-β-D-mannopyranosyl-(1→4)-2-acetamido-3,6-di-O-benzyl-2-deoxy-β-D-glucopyranoside (28)

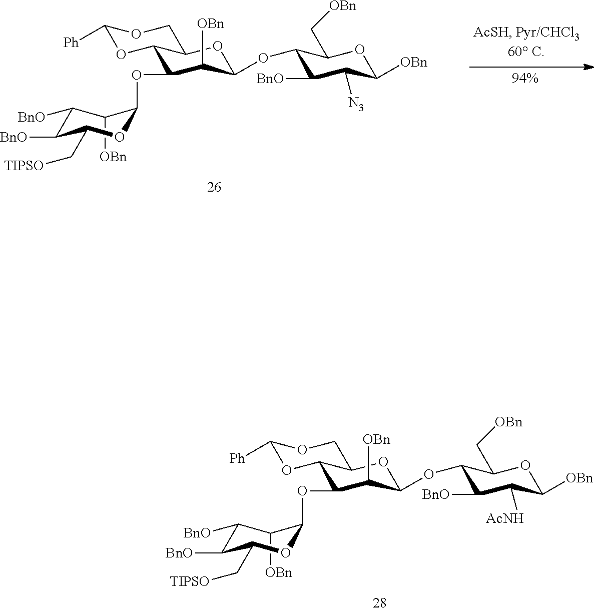

A solution of compound 26 (80.0 mg, 0.057 mmol) in a mixture of AcSH/pyridine/CHCl₃ (0.6 mL/0.4 mL/0.6 mL) was stirred at 60° C. for 18 h. After the completion of the reaction as monitored by TLC, the resulting mixture was concentrated and subjected to flash chromatography on silica gel (hexanes/EtOAc=4:1˜2:1) to afford compound 27 (76.5 mg, 94%) as colorless syrup. R_{f}=0.25 (hexanes/EtOAc=2:1); ¹H NMR (400 MHZ, CDCl₃) δ 7.50-7.48, 7.38-7.16, 7.11-7.09 (40H, m, Ar—H), 5.97 (1H, d, J=8.5 Hz, NH), 5.51 (1H, s), 5.36 (1H, m), 4.98-4.91 (3H, m), 4.81-4.77 (2H, m), 4.72 (1H, d, J=11.3 Hz, PhCH₂), 4.69-4.62 (3H, m), 4.60-4.57 (2H, m), 4.53-4.47 (4H, m), 4.37 (1H, d, J=12.4 Hz), 4.11-4.07 (1H, m), 4.06-3.82 (12H, m), 3.79-3.68 (3H, m), 3.59 (1H, dd, J=10.2 Hz, J=10.2 Hz), 3.19-3.13 (1H, m), 1.69 (3H, s), 1.08 (21H, s); ¹³C NMR (100 MHz, CDCl₃) δ 170.04, 138.83, 138.67, 138.50, 138.28, 138.02, 137.64, 137.40, 129.27, 128.46, 128.35, 128.32, 128.28, 128.21, 128.07, 127.93, 127.86, 127.83, 127.80, 127.67, 127.59, 127.52, 127.49, 127.47, 127.44, 127.26, 126.17, 101.82, 101.52, 99.26, 98.47, 79.71, 79.00, 78.91, 77.22, 75.93, 75.58, 75.28, 75.02, 74.69, 74.59, 74.49, 73.46, 72.94, 71.81, 70.54, 69.56, 68.58, 66.90, 63.44, 53.16, 23.13, 18.09, 18.07, 12.03; MALDI-TOF: [M+Na]⁺ calcd for C₈₅H₁₀₁NNaO₁₆Si, 1442.68; found, 1442.29.

### Benzyl 2,3,4-tri-O-benzyl-α-D-mannopyranosyl-(1→6)-2,4-di-O-benzyl-3-O-p-methoxybenzyl-β-D-mannopyranosyl-(1→4)-2-acetamido-3,6-di-O-benzyl-2-deoxy-β-D-glucopyranoside (29)

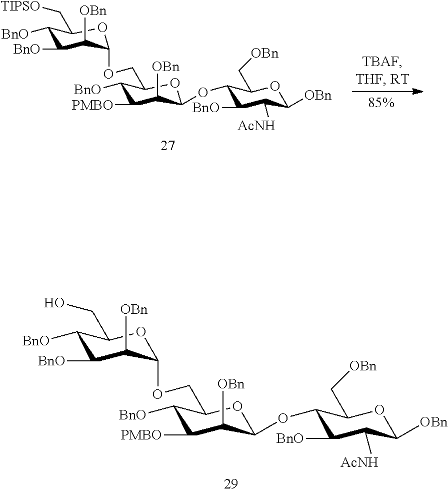

To a solution of compound 27 (65.0 mg, 0.042 mmol) in THF (2.0 mL) was added TBAF (1 M in THF, 210 μL), and the mixture was stirred at room temperature for 20 h. After the completion of the reaction as monitored by TLC, the resulting mixture was concentrated and subjected to flash chromatography on silica gel (hexanes/EtOAc=5:1˜1:2) to afford compound 29 (49.0 mg, 85%) as colorless syrup. R_{f}=0.20 (hexanes/EtOAc=1:1); 1H NMR (400 MHZ, CDCl₃) δ 7.43-7.41, 7.36-7.21 (42H, m, Ar—H), 6.89-6.87 (2H, m, Ar—H), 5.41 (1H, d, J=8.0 Hz, NH), 5.07 (1H, m), 4.99-4.85 (7H, m, PhCH₂), 4.64-4.44 (12H, m), 4.17 (1H, dd, J=7.4 Hz, J=7.4 Hz), 4.08 (1H, dd, J=7.2 Hz, J=7.2 Hz), 3.96 (1H, dd, J=9.4 Hz, J=9.4 Hz), 3.89-3.80 (6H, m), 3.77-3.55 (9H, m), 3.47 (1H, dd, J=2.9 Hz, J=9.2 Hz), 3.39-3.35 (1H, m), 2.59 (1H, m), 1.56 (3H, s); ¹³C NMR (100 MHZ, CDCl₃) δ 170.46, 159.30, 139.14, 138.79, 138.61, 138.49, 138.36, 138.00, 137.84, 130.17, 129.24, 128.47, 128.40, 128.35, 128.31, 128.29, 128.27, 128.24, 128.11, 127.85, 127.84, 127.79, 127.76, 127.72, 127.65, 127.61, 127.56, 127.53, 127.40, 127.34, 113.86, 101.18, 99.24, 97.88, 82.41, 79.89, 75.59, 75.30, 75.19, 75.05, 74.99, 74.76, 74.69, 73.62, 73.56, 72.73, 72.33, 71.86, 71.49, 70.74, 69.46, 67.01, 61.99, 55.30, 23.21; MALDI-TOF: [M+Na]⁺ calcd for C₈₄H₉₁NNaO₁₇, 1408.62; found, 1408.27.

### Benzyl 2,3,4-tri-O-benzyl-α-D-mannopyranosyl-(1→3)-2-O-benzyl-4,6-O-benzylidene-β-D-mannopyranosyl-(1→4)-2-acetamido-3,6-di-O-benzyl-2-deoxy-β-D-glucopyranoside (30)

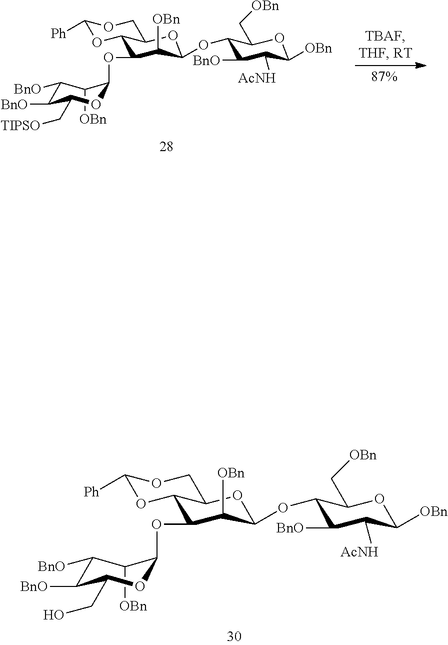

To a solution of compound 28 (76.5 mg, 0.054 mmol) in THF (2.0 mL) was added TBAF (1 M in THF, 269 μL), and the mixture was stirred at room temperature for 20 h. After the completion of the reaction as monitored by TLC, the resulting mixture was concentrated and subjected to flash chromatography on silica gel (hexanes/EtOAc=3:1˜2:3) to afford compound 30 (59.3 mg, 87%) as colorless syrup. R_{f}=0.20 (hexanes/EtOAc=1:1); 1H NMR (400 MHZ, CDCl₃) δ 7.52-7.49, 7.41-7.16, 7.10-7.08 (40H, m, Ar—H), 5.78 (1H, d, J=8.0 Hz, NH), 5.52 (1H, s), 5.35 (1H, m), 5.01-4.91 (4H, m), 4.81-4.77 (2H, m), 4.78 (2H, m), 4.69-4.60 (5H, m), 4.53-4.47 (4H, m), 4.44-4.40 (1H, m), 4.17-3.95 (5H, m), 3.90-3.74 (7H, m), 3.71-3.58 (5H, m), 3.22-3.16 (1H, m), 1.77 (3H, s); ¹³C NMR (100 MHZ, CDCl₃) δ 170.19, 138.86, 138.54, 138.34, 138.06, 137.97, 137.58, 137.45, 129.35, 128.55, 128.38, 128.34, 128.21, 128.19, 127.98, 127.94, 127.88, 127.82, 127.77, 127.71, 127.57, 127.54, 127.50, 126.19, 101.89, 101.55, 99.19, 98.81, 79.64, 78.94, 78.56, 77.86, 75.64, 75.46, 75.15, 74.78, 74.43, 73.70, 73.57, 72.92, 72.16, 71.89, 70.77, 69.11, 68.61, 66.98, 62.52, 55.15, 23.32; MALDI-TOF: [M+Na]⁺ calcd for C₇₆H₈₁NNaO₁₆⁺, 1286.54; found, 1286.20.

### 2,3,4-tri-O-benzyl-6-O-dibenzylphosphonato-α-D-mannopyranosyl-(1→6)-2,4-di-O-benzyl-3-O-p-methoxybenzyl-β-D-mannopyranosyl-(1→4)-2-acetamido-3,6-di-O-benzyl-2-deoxy-β-D-glucopyranoside (31)

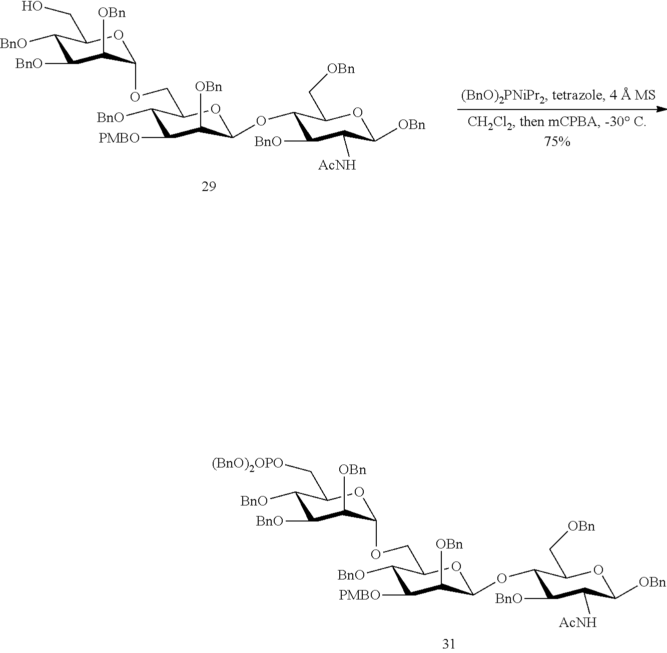

To a solution of compound 29 (38.0 mg, 0.027 mmol) in anhydrous CH₂Cl₂ (1.0 mL) was added activated 4 Å molecular sieves (100 mg) and tetrazole (0.45 M in MeCN, 305 μL) and the mixture was stirred at room temperature for 1.5 h before (BnO)₂PNiPr₂ (37.4 μL) was added. The resulting mixture was further stirred overnight under argon atmosphere at room temperature until the complete disappearance of the starting material. Then the reaction was cooled to −30° C., and mCPBA (77 wt %, 32.5 mg) was added, the reaction mixture was stirred at this temperature for 1 h and then filtered through a Celite pad. The filtrate was diluted with CH₂Cl₂, washed with saturated NaHCO₃(aq.), dried over Na₂SO₄ and concentrated to dryness. The residue was purified by flash chromatography (hexanes/EtOAc=4:1˜2:3) to give compound 31 (33.9 mg, 75%) as colorless syrup. R_{f}=0.20 (hexanes/EtOAc=1:1); ¹H NMR (400 MHz, CDCl₃) δ 7.40-7.38, 7.32-7.15 (52H, m, Ar—H), 6.86-6.84 (2H, m, Ar—H), 5.30 (1H, d, J=7.5 Hz, NH), 5.06-4.88 (8H, m), 4.85-4.82 (3H, m), 4.64-4.36 (12H, m), 4.11 (1H, dd, J=8.2 Hz, J=8.2 Hz), 4.05-3.96 (3H, m), 3.86-3.70 (11H, m), 3.62-3.56 (2H, m), 3.45 (1H, dd, J=2.9 Hz, J=9.3 Hz), 3.40-3.33 (2H, m), 1.53 (3H, s); ¹³C NMR (100 MHZ, CDCl₃) δ 170.27, 159.24, 139.14, 138.66, 138.62, 138.46, 138.40, 138.17, 137.92, 136.19, 136.12, 136.07, 136.00, 130.22, 129.20, 128.45, 128.43, 128.37, 128.34, 128.27, 128.24, 128.20, 128.18, 128.14, 128.02, 127.90, 127.81, 127.70, 127.65, 127.61, 127.51, 127.47, 127.39, 113.81, 100.75, 99.30, 97.91, 82.53, 80.11, 77.77, 77.25, 77.15, 75.36, 75.21, 75.12, 75.01, 74.93, 74.83, 74.74, 74.53, 74.37, 73.97, 73.55, 72.57, 71.92, 71.42, 70.89, 70.77, 69.38, 69.18, 69.13, 69.07, 69.01, 66.74, 66.46, 56.21, 55.29, 29.72, 23.27; 31P NMR (146 MHz, CDCl₃) δ −1.24; MALDI-TOF: [M+Na]⁺ calcd for C₉₈H₁₀₄NNaO₂₀P⁺, 1668.68; found, 1669.05.

### Benzyl 2,3,4-tri-O-benzyl-6-O-dibenzylphosphonato-α-D-mannopyranosyl-(1→3)-2-O-benzyl-4,6-O-benzylidene-β-D-mannopyranosyl-(1→4)-2-acetamido-3,6-di-O-benzyl-2-deoxy-β-D-glucopyranoside (32)

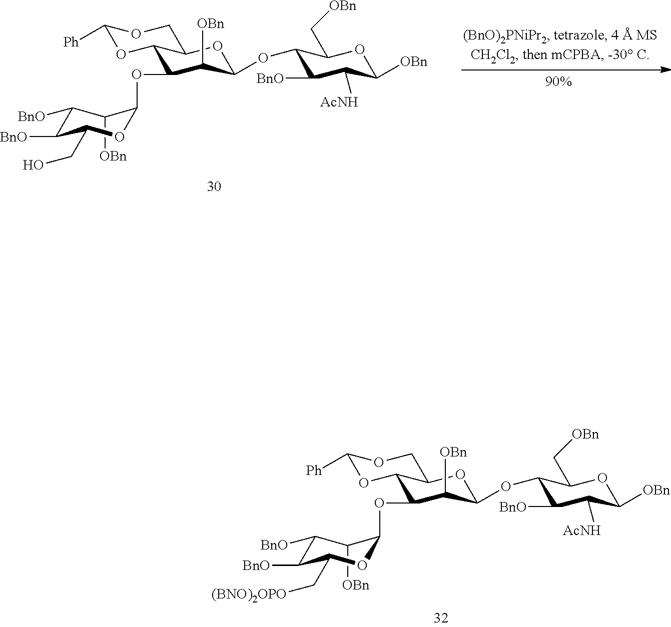

To a solution of compound 30 (58.0 mg, 0.046 mmol) in anhydrous CH₂Cl₂ (2.0 mL) was added activated 4 Å molecular sieves (200 mg) and tetrazole (0.45 M in MeCN, 511 μL) and the mixture was stirred at room temperature for 1.5 h before (BnO)₂PNiPr₂ (62.5 μL) was added. The resulting mixture was further stirred overnight under argon atmosphere at room temperature until the complete disappearance of the starting material. Then the reaction was cooled to −30° C., and mCPBA (77 wt %, 54.4 mg) was added, the reaction mixture was stirred at this temperature for 1 h and then filtered through a Celite pad. The filtrate was diluted with CH₂Cl₂, washed with saturated NaHCO₃(aq.), dried over Na₂SO₄ and concentrated to dryness. The residue was purified by flash chromatography (hexanes/EtOAc=4:1˜2:3) to give compound 32 (63.2 mg, 90%) as colorless syrup. R_{f}=0.20 (hexanes/EtOAc=1:1); ¹H NMR (400 MHZ, CDCl₃) δ 7.49-7.46, 7.40-7.17, 7.14-7.10, 7.04-7.02 (50H, m, Ar—H), 5.83 (1H, d, J=8.1 Hz, NH), 5.49 (1H, s), 5.32 (1H, m), 5.10-5.00 (4H, m), 4.99-4.87 (4H, m), 4.76-4.72 (2H, m), 4.65-4.58 (5H, m), 4.51-4.45 (3H, m), 4.43-4.40 (1H, m), 4.37-4.42 (3H, m), 4.16-4.04 (2H, m), 4.01-3.93 (3H, m), 3.87-3.73 (6H, m), 3.70-3.64 (2H, m), 3.57 (1H, dd, J=10.3 Hz, J=10.3 Hz), 3.11-3.04 (1H, m), 1.76 (3H, s); ¹³C NMR (100 MHz, CDCl₃) δ 170.20, 138.83, 138.43, 138.27, 138.09, 137.98, 137.62, 137.49, 135.96, 135.90, 129.31, 128.57, 128.53, 128.50, 128.46, 128.42, 128.35, 128.33, 128.20, 128.15, 128.03, 127.94, 127.91, 127.87, 127.78, 127.73, 127.65, 127.53, 127.47, 127.42, 126.20, 101.92, 101.24, 99.18, 98.64, 79.59, 78.94, 78.59, 75.64, 75.57, 75.25, 75.05, 74.32, 74.05, 73.51, 73.43, 71.98, 71.78, 70.68, 69.41, 69.35, 69.24, 69.19, 68.61, 66.74, 54.76, 23.29; 31P NMR (146 MHz, CDCl₃) δ −1.08; MALDI-TOF: [M+Na]⁺ calcd for C₉₀H₉₄NNaO₁₉P⁺, 1546.60; found, 1546.01.

### 6-O-phosphonato-α-D-mannopyranosyl-(1→6)-β-D-mannopyranosyl-(1→4)-2-acetamido-2-deoxy-αβ-D-glucopyranoside (33)

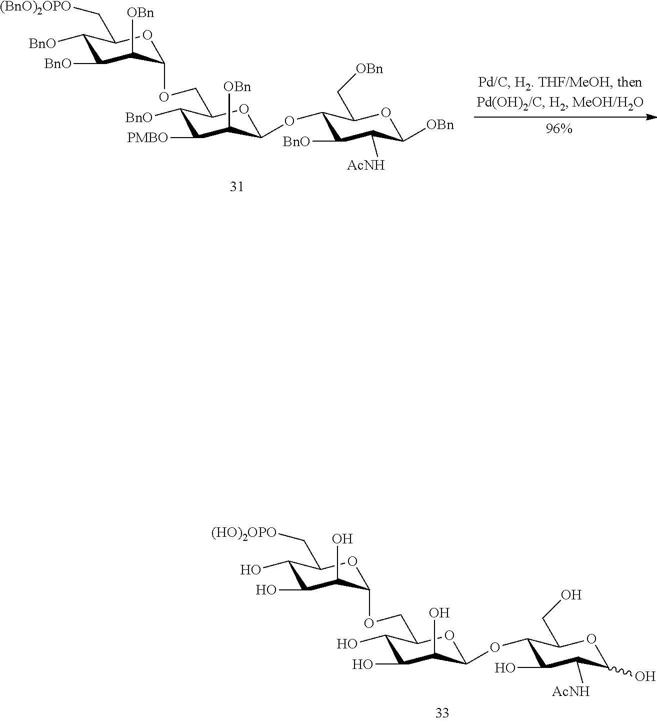

A mixture of compound 31 (60.2 mg, 0.036 mmol) and Pd/C (10 wt. % loading, 30 mg) in MeOH (2.0 mL) and THF (2.0 mL) was stirred under H₂ atmosphere for 21 h. The reaction mixture was filtered through a Celite pad, then concentrated to dryness. The mixture of the residue and Pd(OH)₂/C (20 wt. % loading, 40 mg) in MeOH (2.5 mL) and H₂O (2.5 mL) was stirred under H₂ atmosphere for further 21 h. The reaction mixture was filtered through a Celite pad. The filtrate was concentrated to dryness then dissolved in H₂O and lyophilized. The crude product was purified on a Sephadex G-10 column by elution with H₂O. Fractions containing the product were pooled and lyophilized to give compound 33 (22.0 mg, 96%) as white solid. R_{f}=0.40 (n-BuOH/EtOH/H₂O/AcOH=1:1:1:0.05); ¹H NMR (400 MHZ, D₂O) δ 5.14 (0.63H, d, J=3.2 Hz), 4.84 (1.03H, m), 4.65 (0.39H, m), 4.03-3.98 (3.24H, m), 3.90-3.79 (5.69H, m), 3.75-3.62 (6.60H, m), 3.61-3.53 (4.03H, m), 1.99 (3H, s); ¹³C NMR (100 MHZ, D₂O) δ 174.92, 174.62, 100.55, 100.51, 99.88, 99.84, 94.91, 90.48, 80.28, 79.95, 74.45, 74.30, 72.78, 72.30, 71.88, 71.80, 70.51, 70.46, 70.19, 70.13, 69.93, 69.89, 69.08, 66.70, 66.27, 66.22, 63.59, 60.29, 60.17, 56.01, 53.61, 22.30, 21.98; 31P NMR (146 MHZ, D₂O) δ 1.91 (overlapped signals); HRMS: [M+H]⁺ calcd for C₂₀H₃₇NO₁₉P⁺, 626.1692; found, 626.1690.

### 6-O-phosphonato-α-D-mannopyranosyl-(1→3)-β-D-mannopyranosyl-(1→4)-2-acetamido-2-deoxy-αβ-D-glucopyranoside (34)

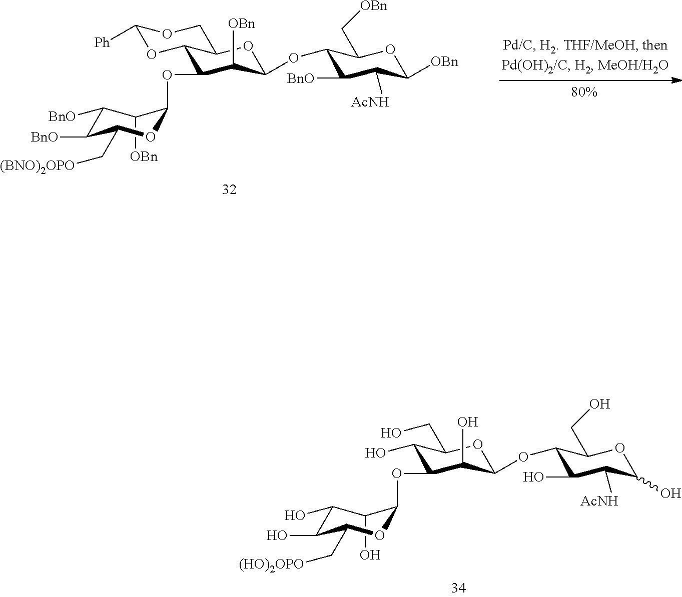

A mixture of compound 32 (53.0 mg, 0.034 mmol) and Pd/C (10 wt. % loading, 30 mg) in MeOH (2.0 mL) and THF (2.0 mL) was stirred under H₂ atmosphere for 21 h. The reaction mixture was filtered through a Celite pad, then concentrated to dryness. The mixture of the residue and Pd(OH)₂/C (20 wt. % loading, 30 mg) in MeOH (2.5 mL) and H₂O (2.5 mL) was stirred under H₂ atmosphere for further 21 h. The reaction mixture was filtered through a Celite pad. The filtrate was concentrated to dryness then dissolved in H₂O and lyophilized. The crude product was purified on a Sephadex G-10 column by elution with H₂O. Fractions containing the product were pooled and lyophilized to give compound 34 (17.4 mg, 80%) as white solid. R_{f}=0.35 (n-BuOH/EtOH/H₂O/AcOH=1:1:1:0.05); ¹H NMR (400 MHZ, D₂O) δ 5.14 (0.60H, d, J=3.4 Hz), 5.06-5.05 (0.98H, m), 4.66-4.64 (0.51H, m), 4.15 (1.06H, m), 4.07-3.96 (3.20H, m), 3.89-3.80 (5.62H, m), 3.76-3.59 (7.68H, m), 3.53-3.49 (0.45H, m), 3.42-3.39 (0.96H, m), 1.98 (3H, s); ¹³C NMR (100 MHZ, D₂O) δ 174.31, 174.02, 102.02, 101.98, 99.44, 94.51, 90.11, 80.15, 80.01, 78.87, 78.49, 75.66, 74.29, 72.05, 71.87, 69.85, 69.80, 69.74, 69.60, 68.70, 66.01, 65.59, 65.53, 63.59, 60.47, 59.87, 59.76, 55.71, 53.26, 21.78, 21.49; 31P NMR (146 MHz, D₂O) δ 1.71 (overlapped signals); HRMS: [M+H]⁺ calcd for C₂₀H₃₇NO₁₉P⁺, 626.1692; found, 626.1690.

### 2-Methyl-[6-O-phosphonato-α-D-mannopyranosyl-(1→6)-β-D-mannopyranosyl-(1→4)-1,2-dideoxy-α-D-glucopyrano]-[2,1-d]-2-oxazoline (6)

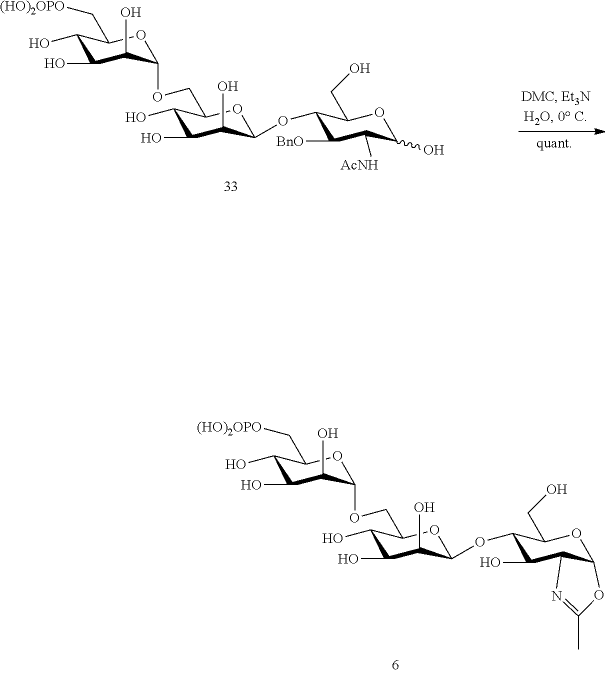

To a solution of compound 33 (8.0 mg, 0.013 mmol) in H₂O (300 μL) were added Et₃N (72.0 μL) and 2-chloro-1,3-dimethylimidazolinium chloride (DMC, 43.4 mg) at 0° C. The reaction mixture was monitored by DIONEX HPAEC-PAD. After 2 h, the HPAEC analysis indicated that the free oligosaccharide was converted into a new oligosaccharide that was eluted earlier than the reducing sugar under the HPAEC condition (see general method). The product was purified by gel filtration on a Sephadex G-10 column that was eluted with 0.1% aq Et₃N to afford compound 6 (7.8 mg, quant.) as white solid after lyophilization with 5 mol. % of NaOH. 1H NMR (400 MHZ, D₂O) δ 6.02 (1H, d, J=7.3 Hz), 4.87 (1H, m), 4.84-4.81 (1H, m), 4.64-4.62 (1H, m), 4.31-4.30 (1H, m), 4.15-4.12 (1H, m), 4.01-3.95 (2H, m), 3.92-3.86 (4H, m), 3.81-3.78 (2H, m), 3.80-3.76 (1H, m), 3.74-3.73 (1H, m), 3.71-3.70 (1H, m), 3.68-3.65 (3H, m), 3.61-3.54 (3H, m), 3.48-3.44 (1H, m), 3.36-3.34 (1H, m), 1.99 (3H, d, J=1.8 Hz); ¹³C NMR (100 MHZ, D₂O) δ 168.55, 101.49, 99.90, 99.83, 77.63, 74.51, 72.96, 72.25, 70.92, 70.48, 70.18, 70.00, 69.06, 66.48, 66.03, 65.81, 65.16, 62.59, 61.77, 12.96; 31P NMR (146 MHz, D₂O) δ 4.45; HRMS: [M+H]⁺ calcd for C₂₀H₃₅NO₁₈P⁺, 608.1586; found, 608.1598.

### 2-Methyl-[6-O-phosphonato-α-D-mannopyranosyl-(1→3)-β-D-mannopyranosyl-(1→4)-1,2-dideoxy-α-D-glucopyrano]-[2,1-d]-2-oxazoline (7)

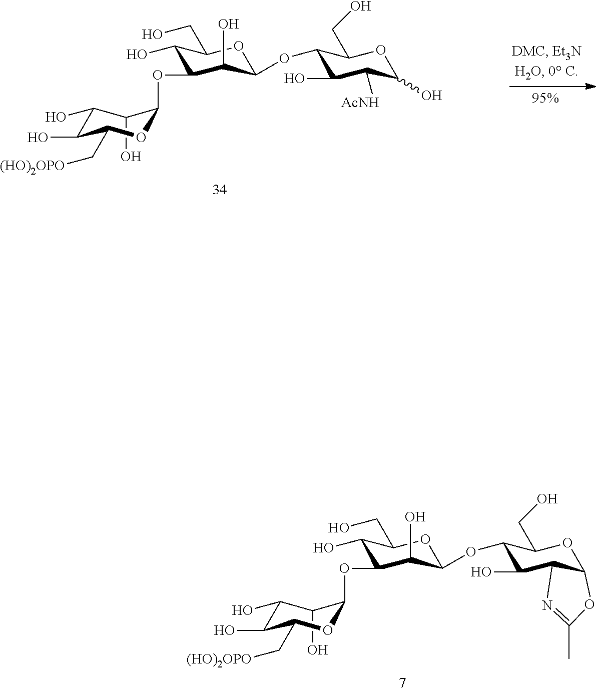

To a solution of compound 34 (8.0 mg, 0.013 mmol) in H₂O (300 μL) were added Et₃N (72.0 L) and 2-chloro-1,3-dimethylimidazolinium chloride (DMC, 43.4 mg) at 0° C. The reaction mixture was monitored by DIONEX HPAEC-PAD. After 2 h, the HPAEC analysis indicated that the free oligosaccharide was converted into a new oligosaccharide that was eluted earlier than the reducing sugar under the HPAEC condition (see general method). The product was purified by gel filtration on a Sephadex G-10 column that was eluted with 0.1% aq Et₃N to afford compound 7 (7.4 mg, 95%) as white solid after lyophilization with 5 mol. % of NaOH. 1H NMR (400 MHZ, D₂₀) δ 6.00 (1H, d, J=7.3 Hz), 5.02 (1H, m), 4.30-4.28 (1H, m), 4.11-4.08 (1H, m), 4.03-4.02 (1H, m), 3.98-3.94 (2H, m), 3.88-3.81 (4H, m), 3.78-3.76 (1H, m), 3.74-3.70 (2H, m), 3.68-3.64 (4H, m), 3.60-3.55 (2H, m), 3.37-3.33 (2H, m), 1.99 (3H, d, J=1.5 Hz); ¹³C NMR (100 MHZ, D₂₀) δ 168.61, 102.51, 101.10, 99.94, 80.35, 77.67, 76.12, 72.82, 72.74, 70.97, 70.13, 70.00, 69.34, 66.19, 66.06, 65.20, 62.65, 62.61, 61.56, 61.04, 13.00; 31P NMR (146 MHz, D₂O) δ 4.45; HRMS: [M+H]⁺ calcd for C₂₀H₃₅NO₁₈P⁺, 608.1586; found, 608.1599.

### Phenyl 3,4-di-O-benzyl-2-O-benzoyl-6-O-triisopropylsilyl-α-D-mannopyranosyl-(1→2)-3,4,6-tri-O-benzyl-1-thio-α-D-mannopyranoside (37)

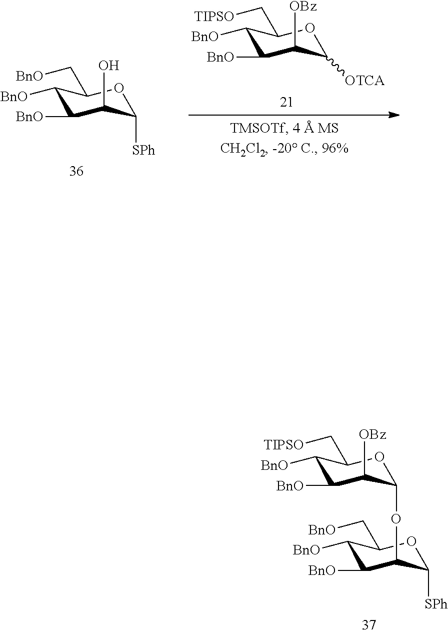

A mixture of trichloroacetimidate donor 21^{[4]} (895 mg, 1.17 mmol), acceptor 36^{[5]} (489 mg, 0.902 mmol) and activated 4 Å molecular sieves (1.0 g) in anhydrous CH₂Cl₂ (10 mL) was stirred at room temperature under argon atmosphere for 1.5 h, and then cooled to −20° C. TMSOTf (16.0 μL, 0.09 mmol) was added. After stirring at −20° ° C. for 0.5 h, the mixture was quenched with triethylamine (20 μL) and filtered then concentrated in vacuo. The residue was purified via silica gel chromatography (hexanes/EtOAc=15:1˜10:1) to give product 37 (995 mg, 96%) as colorless syrup. R_{f}=0.60 (hexanes/EtOAc=5:1); ¹H NMR (400 MHZ, CDCl₃) δ 8.20-8.17, 7.67-7.63, 7.54-7.51, 7.45, 7.41-7.26 (35H, m, Ar—H), 5.82 (1H, m), 5.65 (1H, d, J=1.5 Hz), 5.29 (1H, d, J=1.6 Hz), 4.99-4.93, 4.84-4.78, 4.75-4.65, 4.57-4.53 (10H, m, PhCH₂), 4.37-4.34 (2H, m), 4.21-4.11 (3H, m), 4.05-3.98 (2H, m), 3.92-3.79 (4H, m), 1.15-1.11 (21H, m); ¹³C NMR (100 MHz, CDCl₃) δ 165.63, 138.81, 138.52, 138.46, 138.27, 138.00, 134.44, 133.11, 131.52, 130.16, 130.07, 129.04, 128.54, 128.43, 128.34, 128.32, 128.26, 128.10, 128.09, 128.00, 127.95, 127.83, 127.72, 127.59, 127.56, 127.51, 127.46, 127.41, 99.61, 87.42, 80.25, 78.38, 77.31, 75.92, 75.31, 74.81, 74.04, 73.46, 73.27, 73.04, 72.30, 71.89, 69.39, 69.27, 62.39, 18.13, 18.08, 12.07; MALDI-TOF: [M+Na]⁺ calcd for C₆₉H₈₀NaO₁₁SSi⁺, 1167.51; found, 1167.73.

### Phenyl 2,3,4-tri-O-benzyl-6-O-triisopropylsilyl-α-D-mannopyranosyl-(1→2)-3,4,6-tri-O-benzyl-1-thio-α-D-mannopyranoside (38)

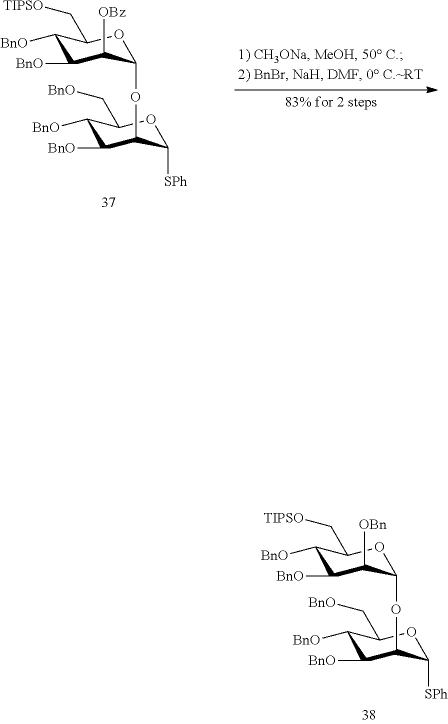

To a solution of compound 37 (1.30 g, 1.136 mmol) in MeOH (12.0 mL) was added sodium methoxide until pH=10, the solution was heated to 50° C. and stirred overnight. After the complete disappearance of the starting material, the solution was concentrated to dryness and dissolved in dry N,N-dimethylformamide (10.0 mL) and cooled to 0° C., sodium hydride (78.4 mg) and benzyl bromide (225 μL) were added successively, and the mixture was slowly warmed to room temperature. After the completion of the reaction as monitored by TLC, MeOH was added to quench the excess sodium hydride. The reaction was diluted with CH₂Cl₂, successively washed with H₂O and brine and dried over anhydrous Na₂SO₄. The residue was purified by flash column chromatography (hexanes/EtOAc=15:1˜10:1) to afford compound 38 (1.059 g, 83% for 2 steps) as colorless syrup. R_{f}=0.60 (hexanes/EtOAc=8:1); 1H NMR (400 MHZ, CDCl₃) δ 7.51-7.48, 7.39-7.27 (35H, m, Ar—H), 5.60 (1H, d, J=1.4 Hz), 5.31 (1H, d, J=2.3 Hz), 4.96-4.90 (2H, m), 4.74-4.69 (3H, m), 4.67 (1H, m), 4.64-4.58 (2H, m), 4.58-4.49 (5H, m), 4.39 (1H, m), 4.32 (1H, m), 4.05 (1H, dd, J=9.4 Hz, J=9.4 Hz), 4.00-3.82 (7H, m), 3.77-3.71 (2H, m), 1.15-1.13 (21H, m); ¹³C NMR (100 MHz, CDCl₃) δ 138.84, 138.78, 138.54, 138.51, 138.42, 137.91, 134.42, 131.20, 128.54, 128.98, 128.56, 128.44, 128.38, 128.33, 128.29, 128.27, 128.15, 128.12, 128.08, 128.03, 128.00, 127.98, 127.78, 127.73, 127.54, 127.42, 127.39, 127.22, 98.91, 87.31, 80.76, 79.81, 77.28, 75.25, 75.11, 75.06, 74.97, 74.76, 74.35, 74.10, 73.29, 72.94, 72.45, 72.30, 72.04, 69.28, 63.01, 18.09, 18.05, 12.06; MALDI-TOF: [M+Na]⁺ calcd for C₆₉H₈₂NaO₁₀SSi⁺, 1153.53; found, 1152.91.

### Benzyl 2,3,4-tri-O-benzyl-6-O-triisopropylsilyl-α-D-mannopyranosyl-(1→2)-3,4,6-tri-O-benzyl-α-D-mannopyranosyl-(1→3)-2-O-benzyl-4,6-O-benzylidene-β-D-mannopyranosyl-(1→4)-2-azido-3,6-di-O-benzyl-2-deoxy-β-D-glucopyranoside (39)

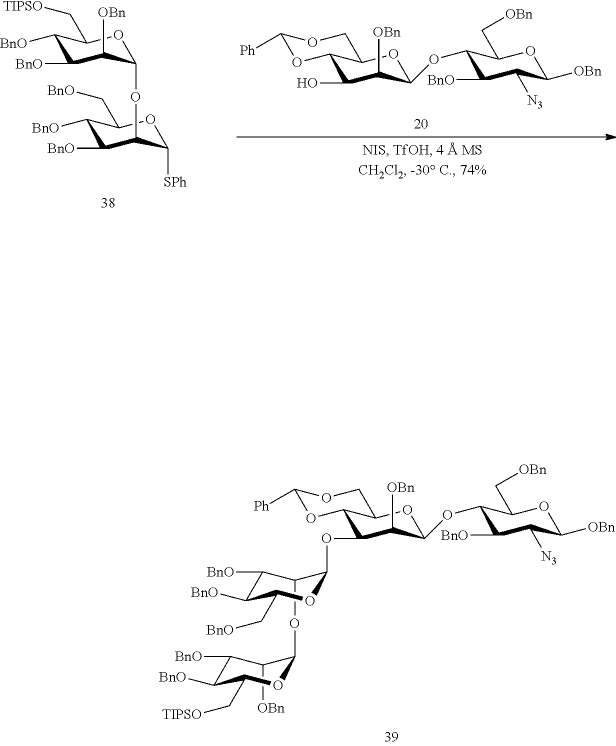

A mixture of compound 38 (180 mg, 0.159 mmol), acceptor 20 (100 mg, 0.123 mmol) and activated 4 Å molecular sieves (450 mg) in anhydrous CH₂Cl₂ (4.5 mL) was stirred at room temperature under argon atmosphere for 1.5 h, and then cooled to −30° C. N-iodosuccinimide (55.2 mg, 0.245 mmol) and TfOH (2.15 μL, 0.025 mmol) were successively added. After stirring at −30° C. for 2 h, the mixture was quenched with triethylamine (10 μL) and filtered then concentrated in vacuo. The residue was purified via silica gel chromatography (hexanes/EtOAc=10:1˜5:1) to give product 39 (166 mg, 74%) as colorless syrup. R_{f}=0.50 (hexanes/EtOAc=4:1); Spectroscopic data were in agreement with literature values.^{[7]} MALDI-TOF: [M+H]⁺ calcd for C₁₁₀H₁₂₆N₃O₂₀Si⁺, 1836.87; found, 1836.44.

### Benzyl 2,3,4-tri-O-benzyl-6-O-triisopropylsilyl-α-D-mannopyranosyl-(1→2)-3,4,6-tri-O-benzyl-α-D-mannopyranosyl-(1→3)-2-O-benzyl-4,6-O-benzylidene-β-D-mannopyranosyl-(1→4)-2-acetamido-3,6-di-O-benzyl-2-deoxy-β-D-glucopyranoside (40)

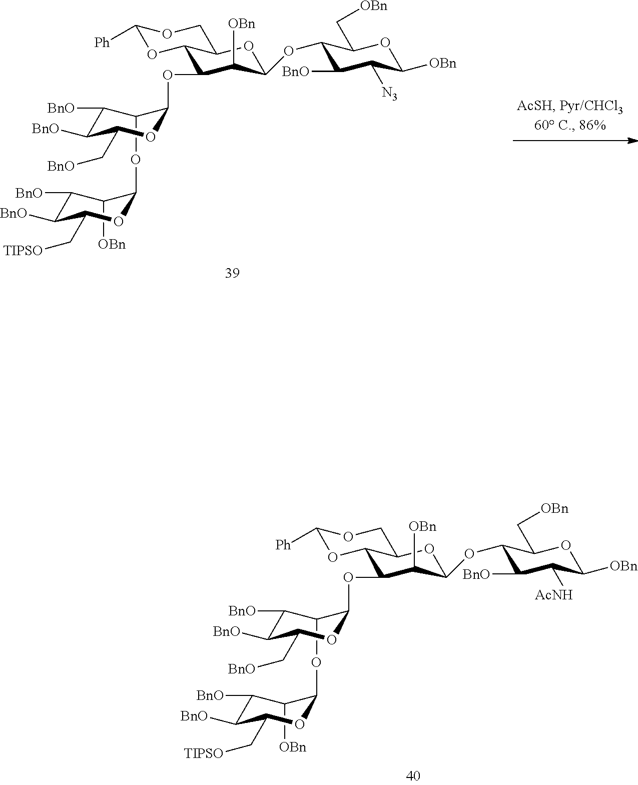

A solution of compound 39 (133.5 mg, 0.073 mmol) in a mixture of AcSH/pyridine/CHCl₃ (0.6 mL/0.4 mL/0.6 mL) was stirred at 60° C. for 18 h. After the completion of the reaction as monitored by TLC, the resulting mixture was concentrated and subjected to flash chromatography on silica gel (hexanes/EtOAc=4:1˜1:1) to afford compound 40 (115.8 mg, 86%) as colorless syrup. R_{f}=0.30 (hexanes/EtOAc=2:1); ¹H NMR (400 MHZ, CDCl₃) δ 7.42-7.23 (54H, m), 7.03 (1H, t, J=7.7 Hz), 5.86 (1H, d, J=8.2 Hz), 5.51 (1H, s), 5.40 (1H, m), 5.29 (1H, m), 4.97-4.88 (4H, m), 4.86 (1H, d, J=4.2 Hz), 4.83 (2H, m), 4.68-4.38 (15H, m), 4.31 (1H, m), 4.19-4.01 (5H, m), 3.98-3.85 (5H, m), 3.82 (1H, m), 3.79-3.53 (10H, m), 3.50 (1H, m), 3.09 (1H, m), 1.75 (3H, s), 1.32-1.28 (3H, m), 1.08 (18H, s); ¹³C NMR (100 MHZ, CDCl₃) δ 170.17, 139.18, 138.89, 138.78, 138.49, 138.46, 138.44, 138.20, 137.97, 137.89, 137.63, 137.16, 128.51, 128.47, 128.40, 128.37, 128.33, 128.28, 128.22, 128.16, 128.10, 128.05, 127.94, 127.90, 127.78, 127.74, 127.70, 127.57, 127.52, 127.34, 127.28, 125.75, 101.52, 101.40, 99.82, 99.20, 97.58, 79.74, 78.92, 78.64, 77.61, 77.27, 75.88, 75.32, 75.19, 75.01, 74.72, 74.57, 74.36, 74.30, 73.72, 73.44, 73.04, 72.26, 72.10, 71.53, 70.90, 70.67, 69.64, 69.26, 68.50, 67.01, 62.38, 60.42, 54.48, 29.73, 23.25, 18.12, 18.08, 12.10; MALDI-TOF: [M+Na]⁺ calcd for C₁₁₂H₁₂₉NNaO₂₁Si⁺, 1876.32; found, 1875.81.

### Benzyl 2,3,4-tri-O-benzyl-α-D-mannopyranosyl-(1→2)-3,4,6-tri-O-benzyl-α-D-mannopyranosyl-(1→3)-2-O-benzyl-4,6-O-benzylidene-β-D-mannopyranosyl-(1→4)-2-acetamido-3,6-di-O-benzyl-2-deoxy-β-D-glucopyranoside (41)

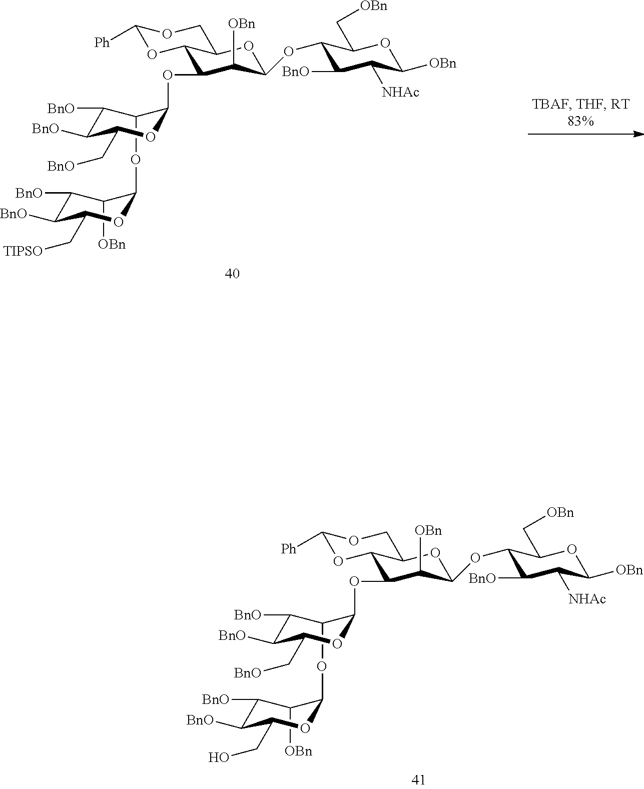

To a solution of compound 40 (115.8 mg, 0.063 mmol) in THF (2.0 mL) was added TBAF (1 M in THF, 313 μL), and the mixture was stirred at room temperature for 23 h. After the completion of the reaction as monitored by TLC, the resulting mixture was concentrated and subjected to flash chromatography on silica gel (hexanes/EtOAc=2:1˜2:3) to afford compound 41 (88.0 mg, 83%) as colorless syrup. R_{f}=0.10 (hexanes/EtOAc=3:2); 1H NMR (400 MHZ, CDCl₃) δ 7.46-7.21 (55H, m), 5.78 (1H, d, J=8.0 Hz), 5.49 (1H, s), 5.25 (1H, d, J=1.5 Hz), 5.04 (1H, m), 4.96 (1H, d, J=6.7 Hz), 4.93-4.79 (6H, m), 4.65-4.48 (15H, m), 4.43 (1H, m), 4.10-4.04 (3H, m), 4.00-3.79 (9H, m), 3.78-3.69 (5H, m), 3.64-3.53 (5H, m), 3.29-3.26 (2H, m), 3.09 (1H, m), 1.75 (3H, s); ¹³C NMR (100 MHZ, CDCl₃) δ 170.19, 138.82, 138.65, 138.60, 138.51, 138.45, 138.37, 138.20, 138.16, 137.88, 137.61, 137.25, 129.12, 128.56, 128.47, 128.44, 128.38, 128.35, 128.29, 128.24, 128.22, 128.18, 128.05, 127.99, 127.95, 127.91, 127.85, 127.82, 127.77, 127.73, 127.65, 127.52, 127.48, 126.08, 101.67, 101.44, 99.97, 99.58, 99.17, 79.58, 78.88, 78.67, 77.70, 77.26, 75.78, 75.60, 75.25, 75.10, 74.92, 74.84, 74.77, 74.43, 73.63, 73.50, 73.38, 72.83, 72.41, 72.32, 72.21, 70.72, 69.55, 69.07, 68.53, 66.93, 61.84, 54.91, 29.73, 23.31; MALDI-TOF: [M+Na]⁺ calcd for C₁₀₃H₁₀₉NNaO₂₁⁺, 1719.98; found, 1719.60.

### Benzyl 2,3,4-tri-O-benzyl-6-O-dibenzylphosphonato-α-D-mannopyranosyl-(1→2)-3,4,6-tri-O-benzyl-α-D-mannopyranosyl-(1→3)-2-O-benzyl-4,6-O-benzylidene-β-D-mannopyranosyl-(1→4)-2-acetamido-3,6-di-O-benzyl-2-deoxy-β-D-glucopyranoside (42)

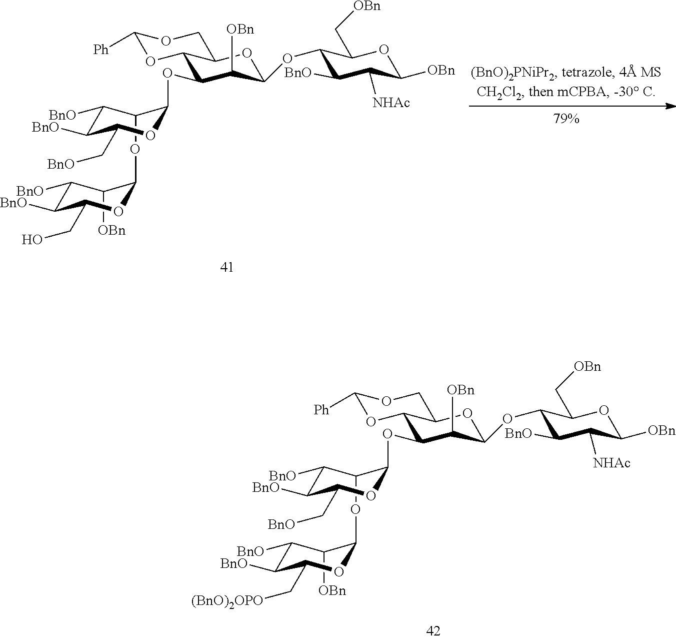

To a solution of compound 41 (73.6 mg, 0.043 mmol) in anhydrous CH₂Cl₂ (2.5 mL) was added activated 4 Å molecular sieves (250 mg) and tetrazole (0.45 M in MeCN, 482 μL) and the mixture was stirred at room temperature for 1.5 h before (BnO)₂PNiPr₂ (58.6 μL) was added. The resulting mixture was further stirred overnight under argon atmosphere at room temperature until the complete disappearance of the starting material. Then the reaction was cooled to −30° C., and mCPBA (77 wt %, 51.5 mg) was added, the reaction mixture was stirred at this temperature for 1 h and then filtered through a Celite pad. The filtrate was diluted with CH₂Cl₂, washed with saturated NaHCO₃(aq.), dried over Na₂SO₄ and concentrated to dryness. The residue was purified by flash chromatography (hexanes/EtOAc=4:1˜2:3) to give compound 42 (67.1 mg, 79%) as colorless syrup. R_{f}=0.20 (hexanes/EtOAc=1:1); ¹H NMR (400 MHZ, CDCl₃) δ 7.41-7.17 (64H, m), 7.06 (1H, t, J=7.5 Hz), 5.91 (1H, d, J=8.2 Hz), 5.44 (1H, s), 5.27 (1H, m), 5.20 (1H, m), 5.06 (1H, dd, J=6.9 Hz, J=11.8 Hz), 5.00-4.79 (10H, m), 4.64-4.43 (14H, m), 4.41-4.36 (2H, m), 4.23 (1H, m), 4.05-3.97 (5H, m), 3.94-3.82 (5H, m), 3.80-3.73 (5H, m), 3.72-3.65 (5H, m), 3.58-3.51 (2H, m), 3.13 (1H, m), 1.72 (3H, s); ¹³C NMR (100 MHZ, CDCl₃) δ 170.15, 138.73, 138.55, 138.39, 138.37, 138.18, 138.13, 138.02, 137.86, 137.64, 137.33, 136.16, 136.08, 136.05, 135.97, 128.55, 128.53, 128.49, 128.44, 128.40, 128.37, 128.32, 128.28, 128.21, 128.18, 128.11, 127.94, 127.93, 127.85, 127.83, 127.79, 127.70, 127.66, 127.58, 127.52, 127.48, 126.11, 101.79, 101.34, 99.62, 99.26, 98.68, 79.95, 79.53, 78.93, 78.77, 77.26, 75.94, 75.44, 75.19, 74.96, 74.71, 74.65, 74.53, 73.91, 73.42, 73.25, 73.09, 72.85, 72.46, 72.06, 71.96, 71.30, 71.23, 70.62, 69.49, 69.15, 69.09, 69.02, 68.97, 68.54, 66.78, 53.94, 29.72, 23.20; 31P NMR (146 MHZ, CDCl₃) δ −1.39; MALDI-TOF: [M+Na]⁺ calcd for C₁₁₇H₁₂₂NNaO₂₄PT, 1980.21; found, 1979.89.

### 6-O-phosphonato-α-D-mannopyranosyl-(1→2)-α-D-mannopyranosyl-(1→3)-β-D-mannopyranosyl-(1→4)-2-acetamido-2-deoxy-αβ-D-glucopyranoside (43)

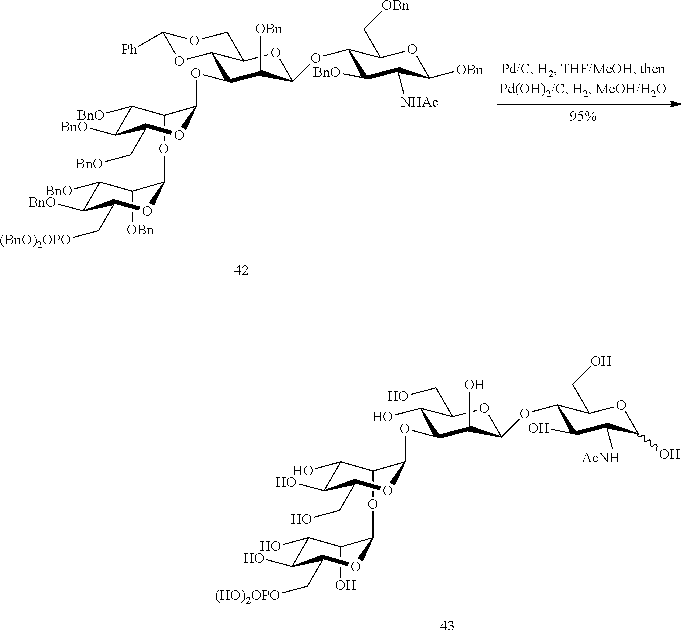

A mixture of compound 42 (67.1 mg, 0.034 mmol) and Pd/C (10 wt. % loading, 40 mg) in MeOH (2.5 mL) and THF (2.5 mL) was stirred under H₂ atmosphere for 21 h. The reaction mixture was filtered through a Celite pad, then concentrated to dryness. The mixture of the residue and Pd(OH)₂/C (20 wt. % loading, 50 mg) in MeOH (4.0 mL) and H₂O (4.0 mL) was stirred under H₂ atmosphere for further 21 h. The reaction mixture was filtered through a Celite pad. The filtrate was concentrated to dryness then dissolved in H₂O and lyophilized. The crude product was purified on a Sephadex G-10 column by elution with H₂O. Fractions containing the product were pooled and lyophilized to give compound 43 (25.6 mg, 95%) as white solid. R_{f}=0.20 (n-BuOH/EtOH/H₂O/AcOH=1:1:1:0.05); ¹H NMR (400 MHZ, D₂O) δ 5.32 (0.96H, s), 5.11 (0.68H, d, J=2.7 Hz), 4.95 (1.17H, s), 4.63-4.61 (0.80H, m), 4.12-4.11 (1.32H, m), 4.01-3.98 (2.47H, m), 3.93-3.90 (2.46H, m), 3.89-3.73 (8.07H, m), 3.73-3.51 (10.71H, m), 3.51-3.38 (1.67H, m), 1.95 (3.00H, s); ¹³C NMR (100 MHZ, D₂O) δ 174.43, 102.39, 100.66, 99.81, 99.74, 94.92, 90.51, 79.89, 79.22, 78.82, 78.50, 76.10, 74.67, 73.40, 72.54, 72.24, 70.43, 70.39, 70.13, 70.00, 69.94, 69.09, 66.96, 66.47, 66.23, 63.43, 61.05, 60.82, 60.14, 60.00, 59.39, 56.13, 53.66, 22.18, 21.88; 31P NMR (146 MHz, D₂O) δ 4.52 (overlapped signals); HRMS: [M+H]⁺ calcd for C₂₆H₄₇NO₂₄P⁺, 788.2220; found, 788.2224.

### 2-Methyl-[6-0)-phosphonato-α-D-mannopyranosyl-(1→2)-α-D-mannopyranosyl-(1→3)-β-D-mannopyranosyl-(1→4)-1,2-dideoxy-α-D-glucopyrano]-[2,1-d]-2-oxazoline (8)

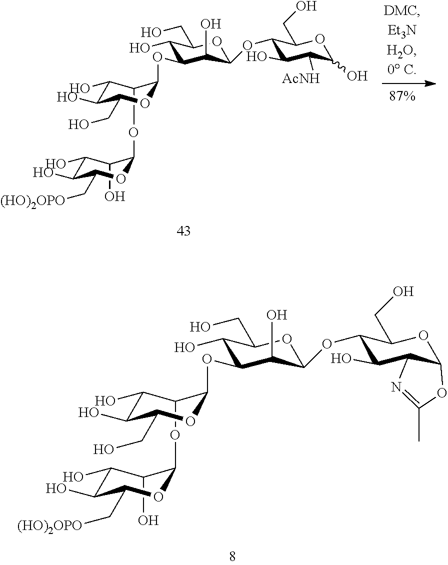

To a solution of compound 43 (5.0 mg, 0.0064 mmol) in H₂O (250 μL) were added Et₃N (35.6 μL) and 2-chloro-1,3-dimethylimidazolinium chloride (DMC, 21.5 mg) at 0° C. The reaction mixture was monitored by DIONEX HPAEC-PAD. After 2 h, the HPAEC analysis indicated that the free oligosaccharide was converted into a new oligosaccharide that was eluted earlier than the reducing sugar under the HPAEC condition (see general method). The product was purified by gel filtration on a Sephadex G-10 column that was eluted with 0.1% aq Et₃N to afford compound 8 (4.3 mg, 87%) as white solid after lyophilization with 5 mol. % of NaOH. 1H NMR (400 MHZ, D₂O) δ 6.02 (1H, m), 5.35 (1H, m), 4.97 (1H, m), 4.31 (1H, m), 4.05-4.03 (2H, m), 4.01-3.99 (1H, m), 3.96-3.89 (4H, m), 3.83-3.76 (3H, m), 3.75-3.64 (9H, m), 3.62-3.54 (3H, m), 3.46-3.41 (1H, m), 3.37-3.33 (1H, m), 2.00 (3H, s); ¹³C NMR (100 MHz, D₂O) δ 167.21, 102.43, 100.92, 100.58, 99.87, 79.58, 78.42, 77.33, 76.18, 73.35, 72.77, 72.69, 71.07, 70.37, 70.13, 70.01, 69.13, 66.96, 66.50, 66.45, 62.83, 61.69, 61.02, 60.98, 12.96; 31P NMR (146 MHZ, D₂O) δ 4.52; HRMS: [M+H]⁺ calcd for C₂₆H₄₅NO₂₃P⁺, 770.2114; found, 770.2124.

### Ethyl 3,4-di-O-benzyl-2-O-benzoyl-6-O-triisopropylsilyl-α-D-mannopyranosyl-(1→6)-2,3,4-tri-O-benzyl-1-thio-α-D-mannopyranoside (45)

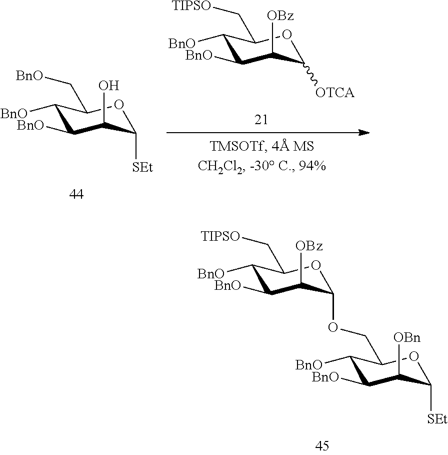

A mixture of trichloroacetimidate donor 21^{[4]} (502 mg, 0.658 mmol), acceptor 44^{[8]} (250 mg, 0.506 mmol) and activated 4 Å molecular sieves (600 mg) in anhydrous CH₂Cl₂ (6.0 mL) was stirred at room temperature under argon atmosphere for 1.5 h, and then cooled to −30° C. TMSOTf (9.25 μL, 0.051 mmol) was added. After stirring at −30° C. for 50 min, the mixture was quenched with triethylamine (50 μL) and filtered then concentrated in vacuo. The residue was purified via silica gel chromatography (hexanes/EtOAc=20:1˜10:1) to give product 45 (522 mg, 94%) as white foam. R_{f}=0.30 (hexanes/EtOAc=10:1); ¹H NMR (400 MHz, CDCl₃) δ 8.16-8.14, 7.62-7.58, 7.48-7.43, 7.38-7.20 (30H, m, Ar—H), 5.75 (1H, m), 5.39 (1H, m), 5.02-4.92 (3H, m), 4.80-4.75 (2H, m), 4.72-4.66 (2H, m), 4.64-4.58 (2H, m), 4.56-4.51 (2H, m), 4.17-4.06 (3H, m), 4.00-3.92 (3H, m), 3.92-3.86 (3H, m), 3.72-3.70 (2H, m), 2.65-2.51 (2H, m), 1.25 (3H, t, J=7.4 Hz), 1.17-1.08 (21H, m); ¹³C NMR (100 MHz, CDCl₃) δ 165.63, 138.98, 138.54, 138.22, 138.14, 138.11, 133.03, 130.17, 130.05, 128.41, 128.31, 128.25, 128.21, 128.19, 128.09, 127.99, 127.87, 127.81, 127.74, 127.69, 127.63, 127.51, 127.40, 98.04, 81.60, 80.52, 78.09, 76.35, 75.10, 74.99, 74.92, 73.96, 72.68, 72.08, 71.98, 71.42, 71.28, 69.01, 66.45, 62.47, 25.29, 18.09, 18.05, 15.04, 12.06; MALDI-TOF: [M+Na]⁺ calcd for C₆₅H₈₀NaO₁₁SSi⁺, 1119.51; found, 1119.17.

### Ethyl 2,3,4-tri-O-benzyl-6-O-triisopropylsilyl-α-D-mannopyranosyl-(1→6)-2,3,4-tri-O-benzyl-1-thio-α-D-mannopyranoside (46)

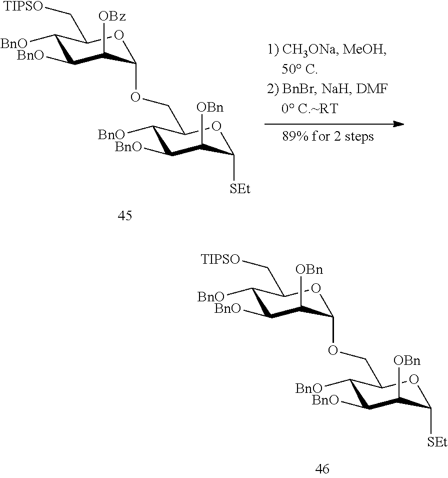

To a solution of compound 45 (300 mg, 0.274 mmol) in MeOH (4.0 mL) was added sodium methoxide until pH=10, the solution was heated to 50° C. and stirred overnight. After the complete disappearance of the starting material, the solution was concentrated to dryness and dissolved in dry N,N-dimethylformamide (3.0 mL) and cooled to 0° C., sodium hydride (27.4 mg) and benzyl bromide (63.5 μL) were added successively, and the mixture was slowly warmed to room temperature. After the completion of the reaction as monitored by TLC, MeOH was added to quench the excess sodium hydride. The reaction was diluted with CH₂Cl₂, successively washed with H₂O and brine and dried over anhydrous Na₂SO₄. The residue was purified by flash column chromatography (hexanes/EtOAc=20:1˜10:1) to afford compound 46 (265 mg, 89% for 2 steps) as colorless syrup. R_{f}=0.60 (hexanes/EtOAc=10:1); 1H NMR (400 MHZ, CDCl₃) δ 7.40-7.23 (30H, m, Ar—H), 5.35 (1H, m), 5.05 (1H, m), 4.97-4.91 (2H, m), 4.73-4.52 (10H, m), 4.10-4.05 (1H, m), 4.04-3.98 (1H, m), 3.95-3.85 (8H, m), 3.71-3.63 (2H, m), 2.63-2.48 (2H, m), 1.23 (3H, dt, J=7.3 Hz, J=0.85 Hz), 1.10-1.07 (21H, m); ¹³C NMR (100 MHZ, CDCl₃) δ 139.06, 138.83, 138.58, 138.54, 138.24, 138.08, 128.40, 128.34, 128.31, 128.23, 128.16, 127.91, 127.87, 127.84, 127.78, 127.75, 127.69, 127.54, 127.47, 127.38, 127.28, 97.76, 81.80, 80.50, 79.73, 76.50, 75.27, 75.06, 74.97, 74.93, 74.70, 73.34, 72.34, 72.20, 72.07, 71.93, 71.66, 65.82, 63.01, 25.27, 18.06, 18.02, 15.05, 12.06; MALDI-TOF: [M+Na]⁺ calcd for C₆₅H₈₂NaO₁₀SSi⁺, 1105.53; found, 1105.15.

### Benzyl 2,3,4-tri-O-benzyl-6-O-triisopropylsilyl-α-D-mannopyranosyl-(1→6)-2,3,4-tri-O-benzyl-α-D-mannopyranosyl-(1→6)-2,4-di-O-benzyl-3-O-p-methoxybenzyl-β-D-mannopyranosyl-(1→4)-2-azido-3,6-di-O-benzyl-2-deoxy-β-D-glucopyranoside (47)

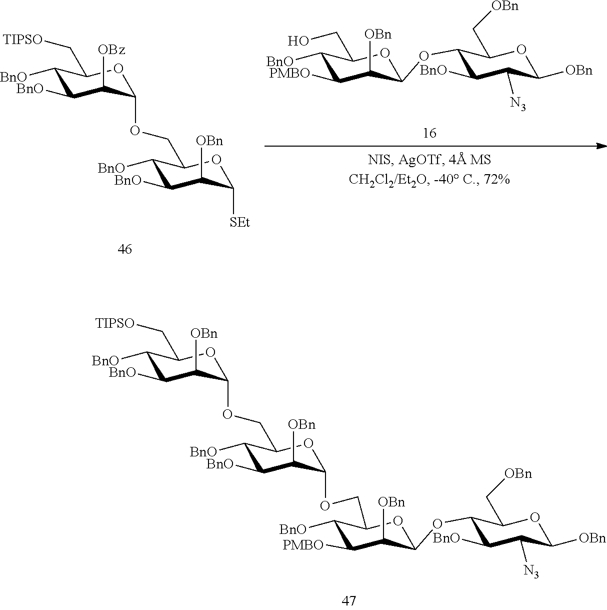

A mixture of compound 46 (120 mg, 0.111 mmol), acceptor 16 (80 mg, 0.085 mmol) and activated 4 Å molecular sieves (400 mg) in anhydrous CH₂Cl₂/Et₂₀ (3 mL/1 mL) was stirred at room temperature under argon atmosphere for 1.5 h, and then cooled to −40° C. N-iodosuccinimide (38.2 mg, 0.170 mmol) and AgOTf (4.4 mg, 0.017 mmol) were successively added. After stirring at −40° C. for 1 h, the mixture was quenched with triethylamine (10 μL) and filtered then concentrated in vacuo. The residue was purified via silica gel chromatography (hexanes/EtOAc=10:1˜4:1) to give the desired product 47 (120 mg, 72%) as colorless oil along with ß isomer (29.5 mg, 17%). R_{f}=0.40 (hexanes/EtOAc=4:1); ¹H NMR (400 MHZ, CDCl₃) δ 7.41-7.17 (57H, m, Ar—H), 6.87-6.85 (2H, m, Ar—H), 5.04-4.98 (2H, m), 4.94-4.76 (8H, m), 4.70-4.40 (15H, m), 4.37-4.28 (3H, m), 4.07-4.02 (1H, m), 4.00-3.64 (16H, m), 3.57-3.42 (4H, m), 3.41-3.33 (4H, m), 3.29-3.26 (1H, m), 1.14-1.00 (21H, m); ¹³C NMR (100 MHZ, CDCl₃) δ 170.64, 158.80, 138.73, 138.59, 138.40, 138.35, 138.16, 138.10, 138.06, 137.96, 137.94, 137.49, 136.44, 129.70, 128.75, 128.71, 128.13, 128.04, 127.97, 127.84, 127.79, 127.77, 127.74, 127.67, 127.58, 127.44, 127.41, 127.36, 127.32, 127.27, 127.13, 127.07, 127.00, 126.96, 126.87, 126.82, 126.77, 126.62, 113.36, 101.05, 100.16, 97.87, 97.71, 82.17, 80.61, 79.44, 78.96, 76.94, 74.91, 74.83, 74.68, 74.60, 74.42, 74.29, 73.98, 73.83, 73.78, 73.70, 73.12, 72.67, 72.28, 71.75, 71.43, 71.02, 70.85, 70.43, 68.21, 66.05, 65.55, 65.16, 62.30, 59.91, 54.79, 20.56, 17.59, 17.55, 13.73, 11.59; MALDI-TOF: [M+Na]⁺ calcd for C₁₁₈H₁₃₅N₃NaO₂₁Si⁺, 1980.93; found, 1981.54.

### Benzyl 2,3,4-tri-O-benzyl-6-O-triisopropylsilyl-α-D-mannopyranosyl-(1→6)-2,3,4-tri-O-benzyl-α-D-mannopyranosyl-(1→6)-2,4-di-O-benzyl-3-O-p-methoxybenzyl-β-D-mannopyranosyl-(1→4)-2-acetamido-3,6-di-O-benzyl-2-deoxy-β-D-glucopyranoside (48)

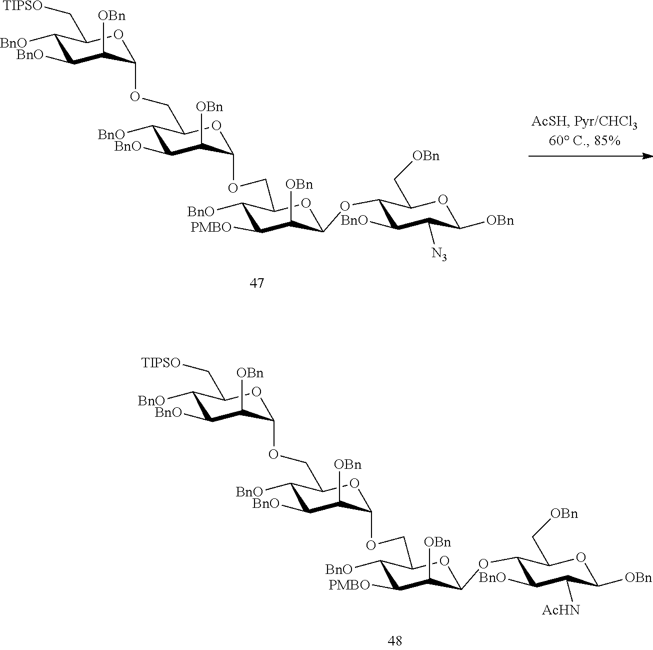

A solution of compound 47 (72.0 mg, 0.037 mmol) in a mixture of AcSH/pyridine/CHCl₃ (0.6 mL/0.4 mL/0.6 mL) was stirred at 60° C. for 20 h. After the completion of the reaction as monitored by TLC, the resulting mixture was concentrated and subjected to flash chromatography on silica gel (hexanes/EtOAc=6:1˜2:1) to afford compound 48 (61.7 mg, 85%) as colorless syrup. R_{f}=0.30 (hexanes/EtOAc=2:1); ¹H NMR (400 MHZ, CDCl₃) δ 7.44-7.16 (57H, m, Ar—H), 6.88-6.86 (2H, m, Ar—H), 5.42 (1H, d, J=7.6 Hz, NH), 5.01-4.84 (10H, m), 4.67-4.38 (18H, m), 4.10-4.02 (3H, m), 3.96-3.94 (1H, m), 3.89-3.77 (13H, m), 3.67-3.64 (2H, m), 3.57-3.53 (2H, m), 3.48-3.42 (2H, m), 3.36-3.33 (1H, m), 3.27-3.25 (1H, m), 1.51 (3H, s), 1.14-1.06 (21H, m); ¹³C NMR (100 MHZ, CDCl₃) δ 169.87, 158.77, 148.48, 138.66, 138.58, 138.42, 138.14, 138.09, 138.05, 138.02, 137.93, 137.60, 137.36, 136.24, 129.70, 128.74, 128.10, 127.98, 127.84, 127.78, 127.70, 127.66, 127.56, 127.53, 127.40, 127.37, 127.33, 127.29, 127.25, 127.18, 127.15, 127.07, 127.01, 126.96, 126.86, 126.81, 126.61, 123.54, 113.34, 100.52, 98.75, 97.61, 97.57, 82.11, 79.75, 78.96, 77.16, 74.92, 74.65, 74.47, 74.39, 74.35, 74.22, 74.13, 74.04, 73.95, 73.37, 73.07, 72.64, 72.29, 71.70, 71.41, 71.32, 70.96, 70.84, 70.39, 68.82, 66.11, 65.10, 62.31, 54.79, 22.63, 17.59, 17.55, 11.59; MALDI-TOF: [M+Na]⁺ calcd for C₁₂₀H₁₃₉NNaO₂₂Si⁺, 1996.95; found, 1997.36.

### Benzyl 2,3,4-tri-O-benzyl-α-D-mannopyranosyl-(1→6)-2,3,4-tri-O-benzyl-α-D-mannopyranosyl-(1→6)-2,4-di-O-benzyl-3-O-p-methoxybenzyl-β-D-mannopyranosyl-(1→4)-2-acetamido-3,6-di-O-benzyl-2-deoxy-β-D-glucopyranoside (49)

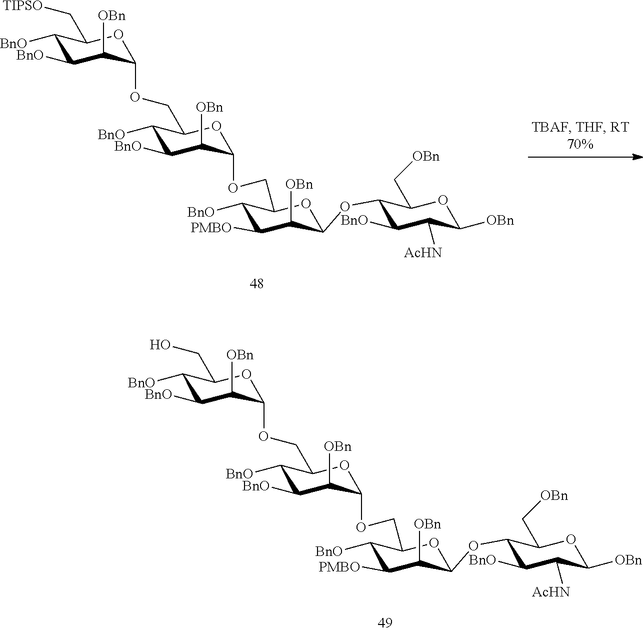

To a solution of compound 48 (61.7 mg, 0.031 mmol) in THF (1.2 mL) was added TBAF (1 M in THF, 174 μL), and the mixture was stirred at room temperature for 20 h. After the completion of the reaction as monitored by TLC, the resulting mixture was concentrated and subjected to flash chromatography on silica gel (hexanes/EtOAc=6:1˜1:1) to afford compound 49 (40.0 mg, 70%) as colorless syrup. R_{f}=0.30 (hexanes/EtOAc=1:1); 1H NMR (400 MHZ, CDCl₃) δ 7.43-7.41, 7.35-7.20 (57H, m, Ar—H), 6.89-6.87 (2H, m, Ar—H), 5.46 (1H, d, J=7.5 Hz, NH), 5.10-5.06 (2H, m), 4.99-4.94 (4H, m), 4.92-4.85 (4H, m), 4.69-4.61 (5H, m), 4.59-4.53 (6H, m), 4.50-4.38 (7H, m), 4.17 (1H, dd, J=8.4 Hz, J=8.4 Hz), 4.05 (1H, dd, J=8.2 Hz, J=8.2 Hz), 4.00-3.94 (2H, m), 3.92-3.76 (12H, m), 3.75-3.69 (3H, m), 3.67-3.62 (3H, m), 3.59-3.55 (1H, m), 3.48-3.45 (1H, m), 3.41-3.32 (2H, m), 3.25-3.22 (1H, m), 1.53 (3H, s); ¹³C NMR (100 MHZ, CDCl₃) δ 170.29, 159.26, 139.25, 138.81, 138.67, 138.65, 138.59, 138.53, 138.45, 138.43, 138.28, 138.12, 137.89, 130.20, 129.23, 128.51, 128.45, 128.39, 128.34, 128.32, 128.30, 128.26, 128.20, 128.15, 128.05, 128.02, 127.96, 127.90, 127.84, 127.75, 127.74, 127.72, 127.69, 127.64, 127.58, 127.55, 127.51, 127.49, 127.46, 127.43, 127.37, 113.84, 100.81, 99.22, 97.90, 82.57, 80.31, 79.22, 77.53, 75.34, 75.30, 75.16, 75.12, 75.05, 74.99, 74.93, 74.79, 74.76, 74.57, 74.15, 73.60, 72.80, 72.45, 72.15, 71.92, 71.76, 71.41, 71.36, 71.05, 69.31, 66.75, 65.31, 62.31, 56.54, 55.31, 29.74, 23.25; MALDI-TOF: [M+Na]⁺ calcd for C₁₁H₁₁₉NNaO₂₂⁺, 1840.81; found, 1841.28.

### Benzyl 2,3,4-tri-O-benzyl-6-O-dibenzylphosphonato-α-D-mannopyranosyl-(1→6)-2,3,4-tri-O-benzyl-α-D-mannopyranosyl-(1→6)-2,4-di-O-benzyl-3-O-p-methoxybenzyl-β-D-mannopyranosyl-(1→4)-2-acetamido-3,6-di-O-benzyl-2-deoxy-β-D-glucopyranoside (50)

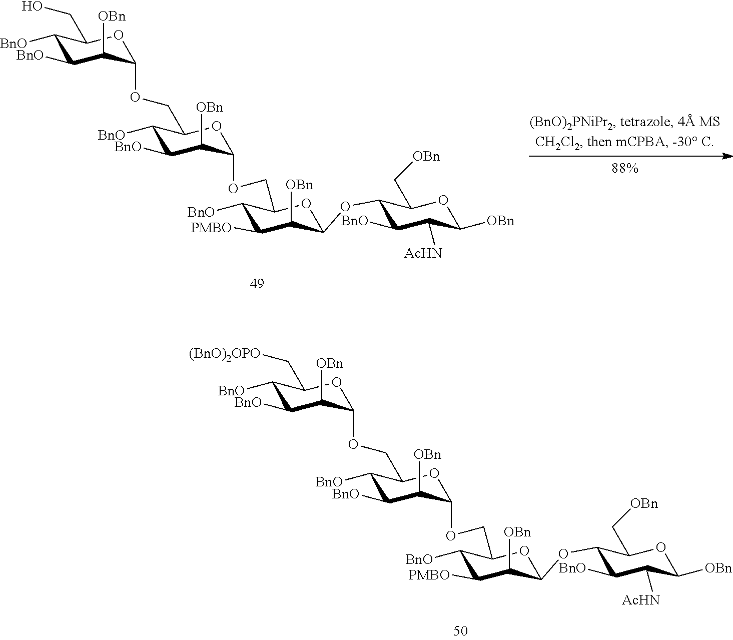

To a solution of compound 49 (40.0 mg, 0.022 mmol) in anhydrous CH₂Cl₂ (1.5 mL) was added activated 4 Å molecular sieves (150 mg) and tetrazole (0.45 M in MeCN, 244 μL) and the mixture was stirred at room temperature for 1.5 h before (BnO)₂PNiPr₂ (37.3 μL) was added. The resulting mixture was further stirred overnight under argon atmosphere at room temperature until the complete disappearance of the starting material. Then the reaction was cooled to −30° C., and mCPBA (77 wt %, 26.1 mg) was added, the reaction mixture was stirred at this temperature for 1 h and then filtered through a Celite pad. The filtrate was diluted with CH₂Cl₂, washed with saturated NaHCO₃(aq.), dried over Na₂SO₄ and concentrated to dryness. The residue was purified by flash chromatography (hexanes/EtOAc=6:1˜3:2) to give compound 50 (40.0 mg, 88%) as colorless syrup. R_{f}=0.15 (hexanes/EtOAc=3:2); ¹H NMR (400 MHZ, CDCl₃) δ 7.43-7.18 (67H, m, Ar—H), 6.89-6.87 (2H, m, Ar—H), 5.45 (1H, d, J=7.6 Hz, NH), 5.13-5.01 (6H, m), 4.99-4.83 (8H, m), 4.67-4.34 (18H, m), 4.26-4.24 (2H, m), 4.18-4.11 (1H, m), 4.06-4.01 (2H, m), 3.97-3.92 (1H, m), 3.89-3.77 (12H, m), 3.69-3.63 (3H, m), 3.60-3.55 (2H, m), 3.49-3.46 (1H, m), 3.40-3.34 (2H, m), 3.26-3.23 (1H, m), 1.53 (3H, s); ¹³C NMR (100 MHz, CDCl₃) δ 170.35, 159.27, 139.25, 138.84, 138.64, 138.61, 138.57, 138.49, 138.45, 138.21, 138.12, 137.91, 130.20, 129.25, 128.49, 128.47, 128.38, 128.34, 128.29, 128.21, 128.17, 128.06, 128.00, 127.91, 127.86, 127.83, 127.80, 127.75, 127.67, 127.51, 127.44, 127.37, 113.84, 100.95, 99.27, 98.00, 97.93, 82.60, 80.22, 79.10, 77.79, 75.31, 75.23, 75.09, 75.00, 74.92, 74.79, 74.55, 74.07, 73.83, 73.58, 72.81, 72.38, 71.89, 71.43, 71.17, 70.96, 69.34, 69.29, 69.19, 69.13, 66.52, 65.56, 56.36, 55.31, 29.74, 23.24; 31P NMR (146 MHZ, CDCl₃) δ −1.13; MALDI-TOF: [M+Na]⁺ calcd for C₁₂₅H₁₃₂NNaO₂₅P⁺, 2100.87; found, 2101.36.

### 6-O-phosphonato-α-D-mannopyranosyl-(1→6)-α-D-mannopyranosyl-(1→6)-β-D-mannopyranosyl-(1→4)-2-acetamido-2-deoxy-αβ-D-glucopyranoside (51)

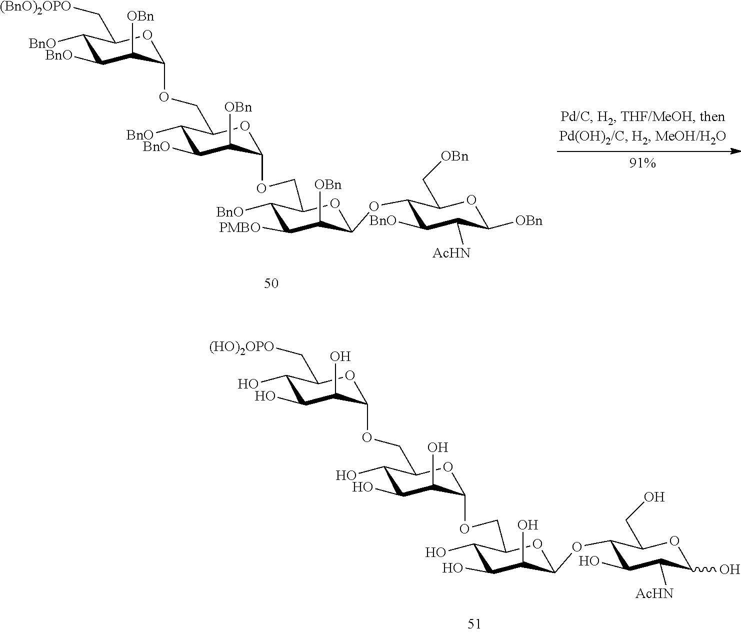

A mixture of compound 50 (40.0 mg, 0.019 mmol) and Pd/C (10 wt. % loading, 20 mg) in MeOH (1.5 mL) and THF (1.5 mL) was stirred under H₂ atmosphere for 21 h. The reaction mixture was filtered through a Celite pad, then concentrated to dryness. The mixture of the residue and Pd(OH)₂/C (20 wt. % loading, 30 mg) in MeOH (2.0 mL) and H₂O (2.0 mL) was stirred under H₂ atmosphere for further 22 h. The reaction mixture was filtered through a Celite pad. The filtrate was concentrated to dryness then dissolved in H₂O and lyophilized. The crude product was purified on a Sephadex G-10 column by elution with H₂O. Fractions containing the product were pooled and lyophilized to give compound 51 (13.7 mg, 91%) as white solid. R_{f}=0.20 (n-BuOH/EtOH/H₂O/AcOH=1:1:1:0.05); ¹H NMR (400 MHZ, D₂O) δ 5.10 (0.67H, m), 4.80 (3.45H, m), 4.62 (0.54H, m), 4.04-3.96 (3.49H, m), 3.91-3.81 (4.95H, m), 3.81-3.73 (4.83H, m), 3.73-3.64 (6.54H, m), 3.64-3.58 (3.02H, m), 3.58-3.45 (3.36H, m), 1.95 (3.00H, s); ¹³C NMR (100 MHZ, D₂O) δ 174.45, 174.13, 100.17, 100.07, 99.38, 99.34, 99.02, 98.93, 94.41, 90.03, 79.85, 79.61, 73.95, 73.77, 72.30, 71.89, 71.23, 71.18, 70.32, 70.22, 70.17, 70.12, 70.02, 69.97, 69.48, 69.41, 68.66, 66.30, 66.06, 65.80, 65.76, 65.15, 65.08, 63.55, 59.82, 59.70, 55.54, 53.13, 46.23, 21.83, 21.51; 31P NMR (146 MHz, D₂O) δ 0.73 (overlapped signals); HRMS: [M+H]⁺ calcd for C₂₆H₄₇NO₂₄P⁺, 788.2220; found, 788.2228.

### 2-Methyl-[6-O-phosphonato-α-D-mannopyranosyl-(1→6)-α-D-mannopyranosyl-(1→6)-β-D-mannopyranosyl-(1→4)-1,2-dideoxy-α-D-glucopyrano]-[2,1-d]-2-oxazoline (9)

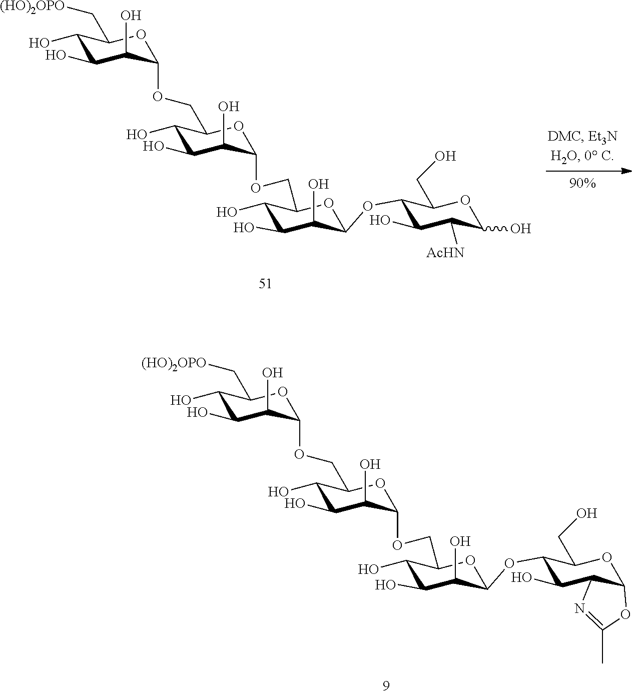

To a solution of compound 51 (7.0 mg, 0.009 mmol) in H₂O (250 μL) were added Et₃N (60 μL) and 2-chloro-1,3-dimethylimidazolinium chloride (DMC, 30 mg) at 0° C. The reaction mixture was monitored by DIONEX HPAEC-PAD. After 2 h, the HPAEC analysis indicated that the free oligosaccharide was converted into a new oligosaccharide that was eluted earlier than the reducing sugar under the HPAEC condition (see general method). The product was purified by gel filtration on a Sephadex G-10 column that was eluted with 0.1% aq Et₃N to afford compound 9 (6.1 mg, 90%) as white solid after lyophilization with 5 mol. % of NaOH. 1H NMR (400 MHZ, D₂O) δ 6.02 (1H, d, J=7.3 Hz), 4.88-4.83 (3H, m), 4.65 (1H, m), 4.31-4.29 (1H, m), 4.14-4.11 (1H, m), 4.01-3.87 (8H, m), 3.86-3.84 (1H, m), 3.80-3.76 (4H, m), 3.75-3.65 (9H, m), 3.60-3.53 (4H, m), 3.49-3.47 (1H, m), 3.37-3.34 (1H, m), 1.99 (3H, d, J=1.6 Hz); ¹³C NMR (100 MHz, D₂O) δ 168.25, 101.49, 99.91, 99.71, 99.67, 77.69, 74.43, 72.90, 72.23, 72.16, 70.89, 70.79, 70.48, 70.26, 70.05, 69.86, 69.18, 69.10, 66.73, 66.58, 66.42, 66.25, 66.10, 65.86, 65.62, 65.13, 62.76, 61.72, 12.97; 31P NMR (146 MHz, D₂O) δ 3.99; HRMS: [M+H]⁺ calcd for C₂₆H₄₅NO₂₃P⁺, 770.2114; found, 770.2133.

### Benzyl 2,3,4-tri-O-benzyl-6-O-triisopropylsilyl-α-D-mannopyranosyl-(1→2)-3,4,6-tri-O-benzyl-α-D-mannopyranosyl-(1→3)-2,4-di-O-benzyl-β-D-mannopyranosyl-(1→4)-2-azido-3,6-di-0)-benzyl-2-deoxy-β-D-glucopyranoside (52)

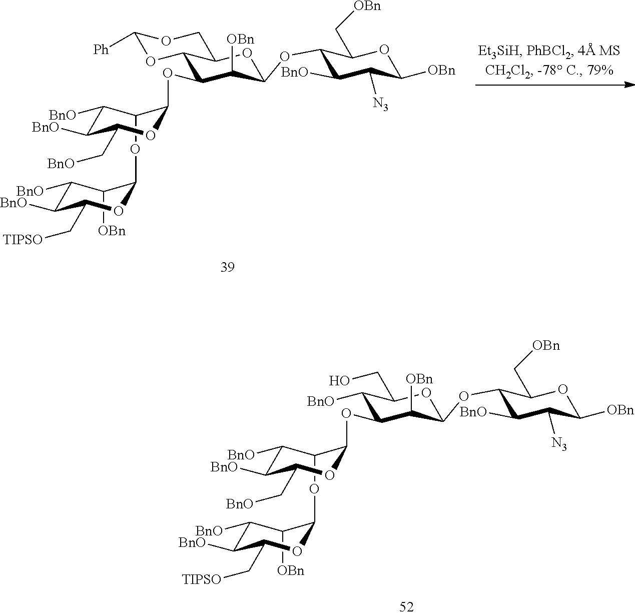

A mixture of compound 39 (130 mg, 0.071 mmol) and activated 4 Å molecular sieves (250 mg) in anhydrous CH₂Cl₂ (2.5 mL) was stirred for 1.5 h at room temperature then cooled to −78° C. Et₃SiH (89.6 μL, 0.565 mmol) and PhBCl₂ (45.6 μL, 0.353 mmol) were added. The resulting mixture was stirred for 2.5 h under argon at −78° C., then Et₃N (135 μL) was added to quench the reaction. The residue was filtered through a Celite pad, diluted with CH₂Cl₂, washed with saturated NaHCO₃(aq) and brine, dried over Na₂SO₄, and concentrated to dryness. Flash chromatography on silica gel (hexane/EtOAc=10:1˜ 3:1) gave compound 52 as colorless syrup (103 mg, 79%). R_{f}=0.30 (hexanes/EtOAc=4:1); Spectroscopic data were in agreement with literature values.^{[7]} MALDI-TOF: [M+Na]⁺ calcd for C₁₁₀H₁₂₇N₃NaO₂₀Si⁺, 1862.30; found, 1862.04.

### Benzyl 2,3,4-tri-O-benzyl-6-O-triisopropylsilyl-α-D-mannopyranosyl-(1→2)-3,4,6-tri-O-benzyl-α-D-mannopyranosyl-(1→3)-[2-O-acetyl-3,4,6-tri-O-benzyl-α-D-mannopyranosyl-(1→6)]-2,4-di-O-benzyl-β-D-mannopyranosyl-(1→4)-2-azido-3,6-di-O-benzyl-2-deoxy-β-D-glucopyranoside (53)

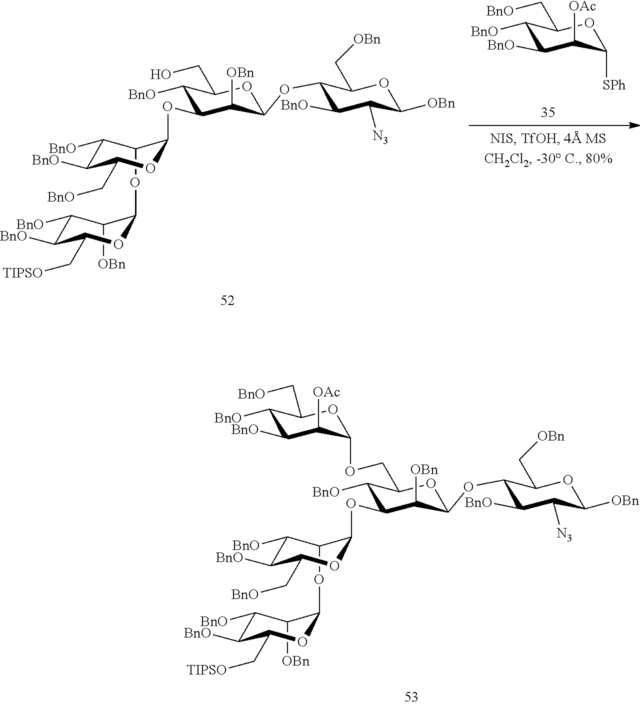

A mixture of compound 35^{[6]} (20 mg, 0.034 mmol), acceptor 52 (34 mg, 0.018 mmol) and activated 4 Å molecular sieves (100 mg) in anhydrous CH₂Cl₂ (1.0 mL) was stirred at room temperature under argon atmosphere for 1.5 h, and then cooled to −30° C. N-iodosuccinimide (14.7 mg, 0.065 mmol) and TfOH (0.38 μL, 0.004 mmol) were successively added. After stirring at −30° C. for 40 min, the mixture was quenched with triethylamine (5 μL) and filtered then concentrated in vacuo. The residue was purified via silica gel chromatography (hexanes/EtOAc=10:1˜4:1) to give the pentasaccharide 53 (34 mg, 80%) as colorless syrup. R_{f}=0.40 (hexanes/EtOAc=4:1); ¹H NMR (400 MHZ, CDCl₃) δ 7.40-7.17 (70H, m), 5.38 (1H, m), 5.23 (1H, m), 5.13 (1H, m), 5.07 (1H, d, J=11.3 Hz), 5.00 (1H, d, J=11.3 Hz), 4.96-4.92 (2H, m), 4.88 (1H, d, J=5.9 Hz), 4.84 (1H, m), 4.80 (1H, d, J=7.2 Hz), 4.78-4.75 (2H, m), 4.72 (1H, m), 4.70-4.62 (4H, m), 4.60 (1H, m), 4.57-4.52 (6H, m), 4.51-4.40 (7H, m), 4.32-4.30 (1H, m), 4.29-4.21 (3H, m), 4.05-3.91 (7H, m), 3.90-3.84 (3H, m), 3.81-3.75 (2H, m), 3.70-3.54 (11H, m), 3.51-3.45 (2H, m), 3.32 (1H, dd, J=9.1 Hz, J=9.1 Hz), 3.25-3.17 (3H, m), 2.08 (3H, s), 1.06 (21H, m); ¹³C NMR (100 MHz, CDCl₃) δ 170.12, 139.07, 138.99, 138.85, 138.74, 138.65, 138.58, 138.54, 138.47, 138.40, 138.06, 138.05, 137.98, 137.95, 137.01, 128.53, 128.43, 128.37, 128.32, 128.29, 128.27, 128.24, 128.21, 128.20, 128.15, 128.07, 128.05, 128.01, 127.91, 127.85, 127.75, 127.71, 127.65, 127.56, 127.51, 127.48, 127.26, 101.51, 101.09, 100.48, 98.56, 97.81, 82.28, 81.14, 80.22, 79.50, 78.71, 78.16, 77.26, 75.16, 75.02, 74.99, 74.87, 74.80, 74.62, 74.52, 74.43, 74.20, 74.05, 73.83, 73.42, 73.35, 73.19, 72.98, 72.44, 72.14, 71.62, 71.41, 70.71, 69.96, 68.56, 68.40, 66.68, 65.70, 62.46, 29.74, 21.07, 18.09, 18.02, 12.06; MALDI-TOF: [M+Na]⁺ calcd for C₁₃₉H₁₅₈N₃O₂₆Si⁺, 2336.85; found, 2336.24.

### Benzyl 2,3,4-tri-O-benzyl-6-O-triisopropylsilyl-α-D-mannopyranosyl-(1→2)-3,4,6-tri-O-benzyl-α-D-mannopyranosyl-(1→3)-[2,3,4,6-tetra-O-benzyl-α-D-mannopyranosyl-(1→6)]-2,4-di-O-benzyl-β-D-mannopyranosyl-(1→4)-2-azido-3,6-di-O-benzyl-2-deoxy-β-D-glucopyranoside (54)

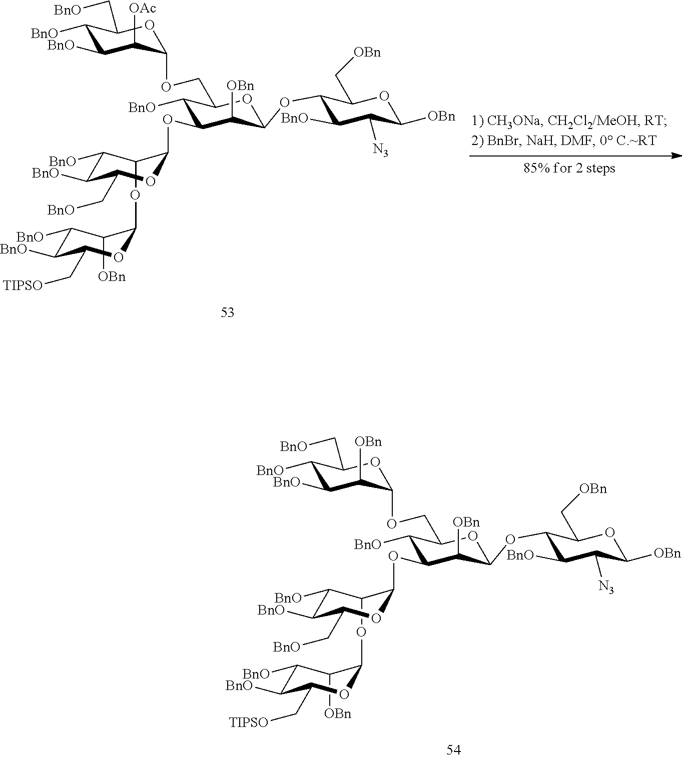

To a solution of compound 53 (34.0 mg, 0.0147 mmol) in MeOH (1.0 mL) and CH₂Cl₂ (1.0 mL) was added sodium methoxide until pH=10, the solution was stirred at room temperature overnight. After the complete disappearance of the starting material, the solution was concentrated to dryness and dissolved in dry N,N-dimethylformamide (2.0 mL) and cooled to 0° C., sodium hydride (3.6 mg, 0.088 mmol) and benzyl bromide (8.5 μL, 0.074 mmol) were added successively, and the mixture was slowly warmed to room temperature. After the completion of the reaction as monitored by TLC, MeOH was added to quench the excess sodium hydride. The reaction was diluted with CH₂Cl₂, successively washed with H₂O and brine and dried over anhydrous Na₂SO₄. The residue was purified by flash column chromatography (hexanes/EtOAc=15:1˜3:1) to afford the compound 54 (29.6 mg, 85% for 2 steps) as colorless syrup. R_{f}=0.70 (hexanes/EtOAc=3:1); ¹H NMR (400 MHZ, CDCl₃) δ 7.39-7.18 (75H, m), 5.24 (1H, m), 5.14 (1H, m), 5.03-4.83 (7H, m), 4.79-4.64 (6H, m), 4.60-4.35 (19H, m), 4.24-4.20 (2H, m), 4.06-3.92 (7H, m), 3.90-3.82 (3H, m), 3.75 (2H, m), 3.69-3.50 (12H, m), 3.47-3.38 (2H, m), 3.29 (1H, dd, J=9.4 Hz, J=9.4 Hz), 3.21-3.15 (2H, m), 1.08-1.03 (21H, m); ¹³C NMR (100 MHz, CDCl₃) δ 139.07, 139.03, 138.84, 138.67, 138.61, 138.55, 138.53, 138.38, 138.20, 137.94, 137.91, 136.96, 128.50, 128.44, 128.37, 128.32, 128.27, 128.24, 128.21, 128.18, 128.15, 128.05, 127.99, 127.91, 127.75, 127.71, 127.67, 127.65, 127.60, 127.57, 127.51, 127.46, 127.40, 127.29, 127.26, 127.15, 127.01, 101.49, 101.34, 100.46, 98.52, 98.07, 82.18, 80.94, 80.27, 79.88, 79.49, 78.94, 77.26, 75.16, 75.05, 74.91, 74.84, 74.77, 74.66, 74.62, 74.50, 74.30, 74.20, 73.83, 73.42, 73.30, 73.18, 72.98, 72.40, 72.30, 72.13, 72.01, 71.59, 71.48, 70.74, 69.95, 68.99, 68.41, 66.32, 65.81, 62.45, 29.73, 18.09, 18.03, 12.06; MALDI-TOF: [M+Na]⁺ calcd for C₁₄H₁₆₁N₃NaO₂₅Si⁺, 2384.94; found, 2384.08.

### Benzyl 2,3,4-tri-O-benzyl-6-O-triisopropylsilyl-α-D-mannopyranosyl-(1→2)-3,4,6-tri-O-benzyl-α-D-mannopyranosyl-(1→3)-[2,3,4,6-tetra-O-benzyl-α-D-mannopyranosyl-(1→6)]-2,4-di-O-benzyl-β-D-mannopyranosyl-(1→4)-2-acetamido-3,6-di-O-benzyl-2-deoxy-β-D-glucopyranoside (55)

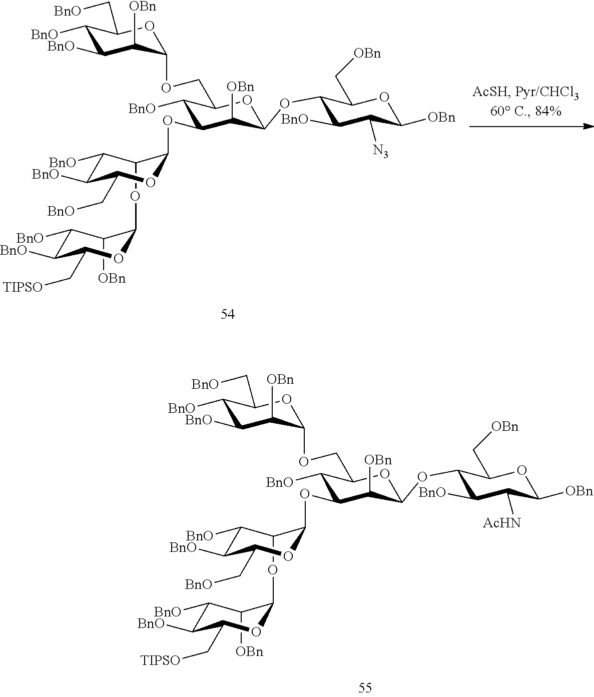

A solution of compound 54 (29.6 mg, 0.013 mmol) in a mixture of AcSH/pyridine/CHCl₃ (0.3 mL/0.2 mL/0.3 mL) was stirred at 60° ° C. for 23 h. After the completion of the reaction as monitored by TLC, the resulting mixture was concentrated and subjected to flash chromatography on silica gel (hexanes/EtOAc=6:1˜2:1) to afford compound 55 (25.0 mg, 84%) as colorless syrup. R_{f}=0.30 (hexanes/EtOAc=2:1); ¹H NMR (400 MHZ, CDCl₃) δ 7.36-7.13 (75H, m), 5.23 (1H, m), 5.17-5.12 (2H, m), 5.03 (1H, d, J=11.9 Hz), 4.96-4.91 (3H, m), 4.90-4.80 (4H, m), 4.76-4.74 (2H, m), 4.69 (1H, d, J=10.7 Hz), 4.65 (1H, m), 4.61-4.42 (19H, m), 4.42-4.37 (2H, m), 4.31 (1H, d, J=12.0 Hz), 4.21 (1H, dd, J=9.6 Hz, J=9.6 Hz), 4.08-3.99 (5H, m), 3.94-3.89 (3H, m), 3.88-3.84 (2H, m), 3.79 (1H, dd, J=9.7 Hz, J=9.7 Hz), 3.74 (2H, m), 3.70-3.62 (9H, m), 3.59-3.56 (1H, m), 3.55-3.53 (1H, m), 3.51-3.49 (2H, m), 3.34 (1H, q, J=7.4 Hz), 3.27 (1H, m), 3.21 (1H, d, J=10.8 Hz), 1.49 (3H, s), 1.08-1.01 (21H, m); ¹³C NMR (100 MHz, CDCl₃) δ 170.07, 139.09, 138.96, 138.84, 138.81, 138.70, 138.67, 138.57, 138.54, 138.35, 138.14, 138.09, 137.97, 137.91, 128.42, 128.38, 128.30, 128.27, 128.23, 128.19, 128.14, 128.05, 128.02, 127.97, 127.81, 127.75, 127.71, 127.70, 127.65, 127.61, 127.56, 127.49, 127.43, 127.28, 101.45, 100.92, 99.16, 98.56, 97.99, 82.06, 80.29, 79.52, 78.87, 77.77, 77.26, 75.33, 75.12, 75.03, 74.90, 74.72, 74.22, 73.91, 73.81, 73.42, 73.19, 72.95, 72.40, 72.29, 72.14, 71.87, 71.82, 71.62, 70.83, 69.71, 68.99, 68.85, 66.56, 62.50, 56.12, 31.96, 29.73, 23.29, 22.72, 18.09, 18.03, 12.06; MALDI-TOF: [M+Na]⁺ calcd for C₁₄₆H₁₆₅NNaO₂₆Si⁺, 2399.13; found, 2398.92.

### Benzyl 2,3,4-tri-O-benzyl-α-D-mannopyranosyl-(1→2)-3,4,6-tri-O-benzyl-α-D-mannopyranosyl-(1→3)-[2,3,4,6-tetra-O)-benzyl-α-D-mannopyranosyl-(1→6)]-2,4-di-O-benzyl-β-D-mannopyranosyl-(1→4)-2-acetamido-3,6-di-O-benzyl-2-deoxy-β-D-glucopyranoside (56)

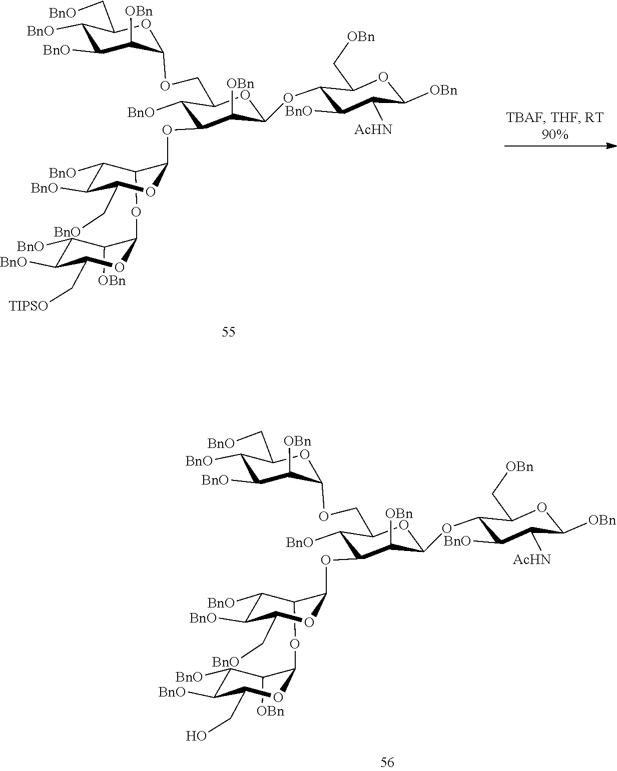

To a solution of compound 55 (25.0 mg, 0.011 mmol) in THF (1.0 mL) was added TBAF (1 M in THF, 84.2 μL), and the mixture was stirred at room temperature for 18 h. After the completion of the reaction as monitored by TLC, the resulting mixture was concentrated and subjected to flash chromatography on silica gel (hexanes/EtOAc=4:1˜1:1) to afford compound 56 (21.0 mg, 90%) as colorless syrup. R_{f}=0.10 (hexanes/EtOAc=2:1); 1H NMR (400 MHZ, CDCl₃) δ 7.37-7.15 (75H, m), 5.20 (1H, m), 5.15 (1H, d, J=7.5 Hz), 5.03-5.01 (2H, m), 4.96-4.82 (7H, m), 4.76 (1H, d, J=11.8 Hz), 4.71 (1H, d, J=11.7 Hz), 4.63-4.40 (21H, m), 4.33 (1H, d, J=12.1 Hz), 4.08-3.96 (4H, m), 3.95-3.86 (5H, m), 3.85-3.73 (6H, m), 3.72-3.63 (6H, m), 3.58-3.50 (5H, m), 3.40-3.33 (1H, m), 3.32-3.25 (2H, m), 1.95 (1H, m), 1.51 (3H, s); ¹³C NMR (100 MHz, CDCl₃) § 170.04, 139.33, 138.92, 138.82, 138.69, 138.65, 138.58, 138.56, 138.49, 138.39, 138.37, 138.19, 138.09, 137.98, 137.89, 128.53, 128.46, 128.36, 128.31, 128.27, 128.22, 128.21, 128.19, 128.15, 127.99, 127.81, 127.79, 127.70, 127.60, 127.51, 127.47, 127.43, 127.33, 101.26, 100.91, 100.09, 99.17, 98.04, 82.19, 80.28, 79.62, 79.53, 78.85, 77.77, 77.26, 75.77, 75.26, 75.20, 75.01, 74.92, 74.85, 74.75, 73.90, 73.47, 73.41, 73.22, 72.99, 72.80, 72.61, 72.48, 72.34, 72.27, 71.96, 71.86, 70.81, 69.54, 68.96, 68.89, 66.55, 62.21, 56.12, 29.73, 23.29; MALDI-TOF: [M+Na]⁺ calcd for C₁₃₇H₁₄₅NNaO₂₆⁺, 2242.99; found, 2243.15.

### Benzyl 2,3,4-tri-O-benzyl-6-O-dibenzylphosphonato-α-D-mannopyranosyl-(1→2)-3,4,6-tri-O-benzyl-α-D-mannopyranosyl-(1→3)-[2,3,4,6-tetra-O-benzyl-α-D-mannopyranosyl-(1→6)]-2,4-di-O-benzyl-β-D-mannopyranosyl-(1→4)-2-acetamido-3,6-di-O-benzyl-2-deoxy-β-D-glucopyranoside (57)

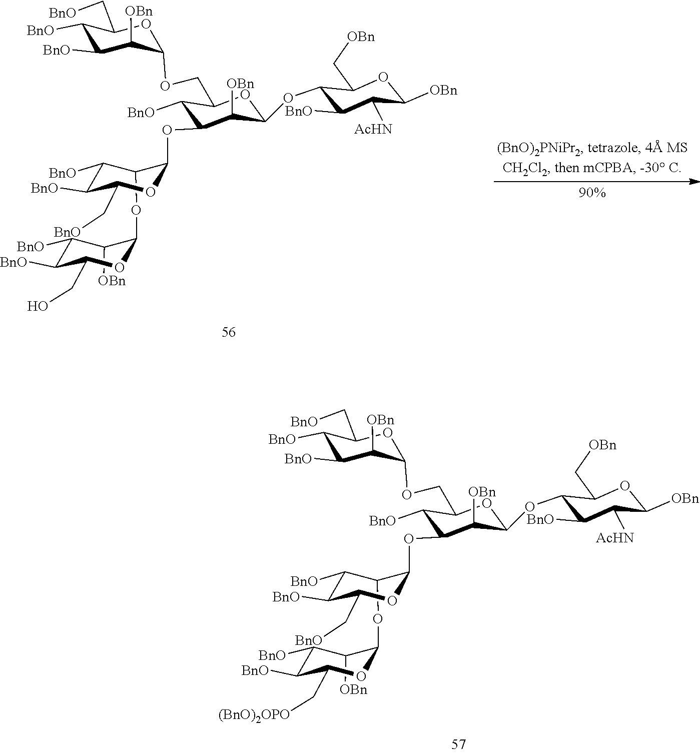

To a solution of compound 56 (21.0 mg, 0.0095 mmol) in anhydrous CH₂Cl₂ (1.0 mL) was added activated 4 Å molecular sieves (100 mg) and tetrazole (0.45 M in MeCN, 105 μL) and the mixture was stirred at room temperature for 1.5 h before (BnO)₂PNiPr₂ (12.8 μL) was added. The resulting mixture was further stirred overnight under argon atmosphere at room temperature until the complete disappearance of the starting material. Then the reaction was cooled to −30° C., and mCPBA (77 wt %, 11.2 mg) was added, the reaction mixture was stirred at this temperature for 1 h and then filtered through a Celite pad. The filtrate was diluted with CH₂Cl₂, washed with saturated NaHCO₃(aq.), dried over Na₂SO₄ and concentrated to dryness. The residue was purified by flash chromatography (hexanes/EtOAc=4:1˜1:1) to give compound 57 (21.0 mg, 90%) as colorless syrup. R_{f}=0.10 (hexanes/EtOAc=2:1); ¹H NMR (400 MHZ, CDCl₃) δ 7.34-7.13 (85H, m), 5.28 (1H, d, J=7.7 Hz), 5.12-5.07 (2H, m), 5.05-5.02 (2H, m), 4.98-4.93 (5H, m), 4.90-4.79 (6H, m), 4.74-4.71 (3H, m), 4.60-4.58 (2H, m), 4.56-4.53 (5H, m), 4.51-4.43 (12H, m), 4.40-4.31 (3H, m), 4.15-4.13 (2H, m), 4.07-3.97 (7H, m), 3.93-3.85 (6H, m), 3.84-3.78 (3H, m), 3.75-3.68 (6H, m), 3.60-3.55 (4H, m), 3.52-3.51 (1H, m), 3.44 (1H, q, J=7.5 Hz), 3.39-3.34 (1H, m), 3.26 (1H, m), 1.50 (3H, s); ¹³C NMR (100 MHz, CDCl₃) δ 169.63, 138.74, 138.44, 138.36, 138.21, 138.14, 138.04, 137.97, 137.91, 137.84, 137.74, 137.61, 137.51, 137.42, 135.51, 135.46, 135.41, 135.36, 132.59, 129.17, 128.22, 128.06, 127.99, 127.97, 127.88, 127.87, 127.83, 127.82, 127.79, 127.74, 127.70, 127.67, 127.57, 127.55, 127.50, 127.42, 127.35, 127.30, 127.25, 127.24, 127.17, 127.09, 127.03, 126.99, 126.93, 126.89, 126.78, 126.70, 100.67, 100.33, 98.96, 98.65, 97.52, 81.75, 79.78, 79.23, 79.05, 78.53, 77.00, 74.91, 74.77, 74.71, 74.53, 74.49, 74.36, 74.27, 74.24, 74.18, 74.12, 73.57, 73.12, 72.86, 72.84, 72.69, 72.26, 71.90, 71.82, 71.72, 71.58, 71.44, 71.33, 70.21, 69.00, 68.82, 68.69, 68.65, 68.61, 68.57, 68.40, 66.87, 66.83, 66.24, 66.04, 54.88, 29.21, 22.70; 31P NMR (146 MHZ, CDCl₃) δ −1.33; MALDI-TOF: [M+Na]⁺ calcd for C₁₅₁H₁₅₈NNaO₂₉P⁺, 2503.05; found, 2502.94.

### 6-O-phosphonato-α-D-mannopyranosyl-(1→2)-α-D-mannopyranosyl-(1→3)-[α-D-mannopyranosyl-(1→6)]-β-D-mannopyranosyl-(1→4)-2-acetamido-2-deoxy-αβ-D-glucopyranoside (58)

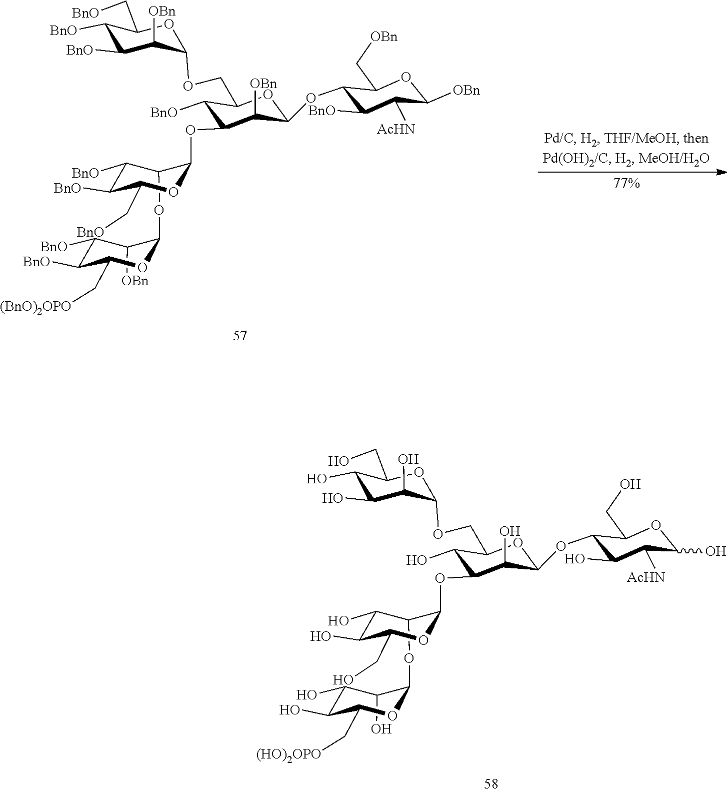

A mixture of compound 57 (44.6 mg, 0.018 mmol) and Pd/C (10 wt. % loading, 20 mg) in MeOH (1.5 mL) and THF (1.5 mL) was stirred under H₂ atmosphere for 21 h. The reaction mixture was filtered through a Celite pad, then concentrated to dryness. The mixture of the residue and Pd(OH)₂/C (20 wt. % loading, 30 mg) in MeOH (2.0 mL) and H₂O (2.0 mL) was stirred under H₂ atmosphere for further 22 h. The reaction mixture was filtered through a Celite pad. The filtrate was concentrated to dryness then dissolved in H₂O and lyophilized. The crude product was purified on a Sephadex G-10 column by elution with H₂O. Fractions containing the product were pooled and lyophilized to give compound 58 (13.1 mg, 77%) as white solid. R_{f}=0.30 (n-BuOH/EtOH/H₂O/AcOH=1:1:1:0.05); ¹H NMR (400 MHZ, D₂O) δ 5.34 (1.00H, m), 5.15 (1H, d, J=3.2 Hz), 4.97 (1.14H, m), 4.87 (1.16H, m), 4.76-4.75 (1.80H, m), 4.66-4.65 (1.00H, m), 4.17-4.15 (1.19H, m), 4.03-4.01 (2.51H, m), 3.99-3.96 (1.55H, m), 3.95-3.89 (3.98H, m), 3.88-3.76 (11.96H, m), 3.74-3.64 (11.78H, m), 3.62-3.55 (3.85H, m), 1.99 (3.00H, m); ¹³C NMR (100 MHZ, D₂O) δ 174.42, 174.13, 102.00, 100.33, 99.73, 99.26, 94.50, 90.07, 79.69, 79.63, 79.29, 78.28, 74.11, 73.75, 73.01, 72.27, 72.12, 72.07, 71.82, 69.99, 69.92, 69.77, 69.56, 69.51, 68.65, 66.60, 66.45, 66.09, 65.85, 65.62, 65.53, 63.10, 60.68, 60.56, 59.76, 59.63, 55.67, 53.20, 21.85, 21.54; 31P NMR (146 MHz, D₂O) δ 2.94 (overlapped signals); HRMS: [M+H]⁺ calcd for C32H57NO29P⁺, 950.2748; found, 950.2755.

### 2-Methyl-[6-O-phosphonato-α-D-mannopyranosyl-(1→2)-α-D-mannopyranosyl-(1→3)-[α-D-mannopyranosyl-(1→6)]-β-D-mannopyranosyl-(1→4)-1,2-dideoxy-α-D-glucopyrano]-[2,1-d]-2-oxazoline (10)

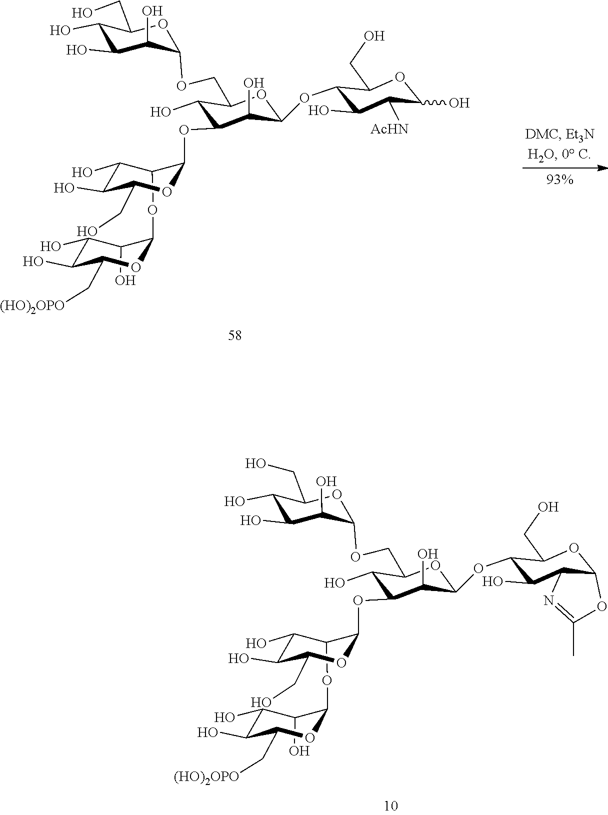

To a solution of compound 58 (8.0 mg, 0.0084 mmol) in H₂O (250 μL) were added Et₃N (47.2 μL) and 2-chloro-1,3-dimethylimidazolinium chloride (DMC, 28.6 mg) at 0° C. The reaction mixture was monitored by DIONEX HPAEC-PAD. After 2 h, the HPAEC analysis indicated that the free oligosaccharide was converted into a new oligosaccharide that was eluted earlier than the reducing sugar under the HPAEC condition (see general method). The product was purified by gel filtration on a Sephadex G-10 column that was eluted with 0.1% aq Et₃N to afford compound 10 (7.3 mg, 93%) as white solid after lyophilization with 5 mol. % of NaOH. 1H NMR (400 MHZ, D₂O) δ 6.01 (1H, d, J=7.3 Hz), 5.32 (1H, m), 4.94 (1H, m), 4.88 (1H, m), 4.32-4.31 (1H, m), 4.12-4.11 (1H, m), 4.04-3.98 (3H, m), 3.95-3.85 (6H, m), 3.84-3.80 (2H, m), 3.79-3.73 (5H, m), 3.71-3.62 (8H, m), 3.61-3.55 (4H, m), 3.35-3.32 (1H, m), 1.99 (3H, d, J=1.7 Hz); ¹³C NMR (100 MHZ, D₂O) δ 167.65, 102.48, 101.25, 100.69, 99.92, 99.59, 80.03, 78.74, 77.78, 74.31, 73.33, 72.68, 72.59, 70.96, 70.50, 70.37, 70.13, 69.98, 69.95, 69.89, 69.05, 66.95, 66.84, 66.43, 66.20, 65.73, 65.03, 62.99, 61.67, 61.00, 60.94, 12.95; 31P NMR (146 MHz, D₂O) δ 4.02; HRMS: [M+H]⁺ calcd for C32H55NO28P⁺, 932.2643; found, 932.2669.

### Example 10: Synthesis of Glycopeptides

GlcNAc-Peptide Derived from rhGAA Containing N470 Glycosite

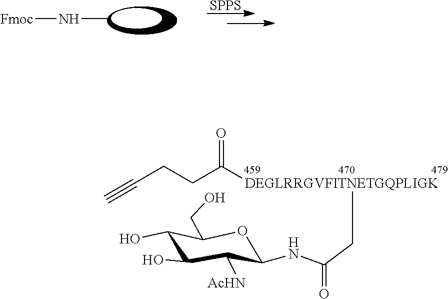

The GlcNAc-Peptide was obtained from SPPS. Synthesis was based on Fmoc chemistry using Rink Amide AM resin (0.66 mmol/g) on a 0.1 mmol scale. Couplings were performed using 5 equiv. of Fmoc-protected amino acids, 5 equiv. of HOBT and 5 equiv. of DIC in DMF. The GlcNAc-Asn building block (3 equiv.) was coupled to the growing peptide at 90° ° C. with a 50 Hz MW power for 10 min, Fmoc-Arg(Pbf)-OH was double coupled (RT without MW for 25 min, followed by 90° C. with 50 Hz MW power for 2 min), and all other amino acids were coupled at 90° ° C. with 50 Hz MW power for 2 min. Fmoc deprotection was carried out with 20% piperidine in DMF containing 0.1 M HOBt. Upon completion of the sequence, 4-pentynoic acid was coupled at the N-terminus to install the alkyne group. The resin was washed with DMF (3×) and DCM (3×) then cleavage was carried out using cocktail R (TFA/Thioanisole/Ethanedithiol/Anisole=90/5/3/2) treatment for 2 h. The resin was then filtered and the solution was added to cold diethyl ether for precipitation. The crude peptide was purified on preparative RP-HPLC to afford the peptide (99.1 mg, 38% yield over all steps). ESI-MS: Calcd., M=2583.89; found (m/z): 646.90 [M+4H]⁴⁺+, 861.43 [M+3H]³⁺, 1292.36 [M+2H]²⁺. Deconvolution of the ESI-MS: M=2583.4; RP-HPLC retention time, t_{R}=18.7 min (gradient, 5-40% aq MeCN containing 0.1% FA for 30 min; flow rate, 1.0 mL/min).

### Glycopeptide 59

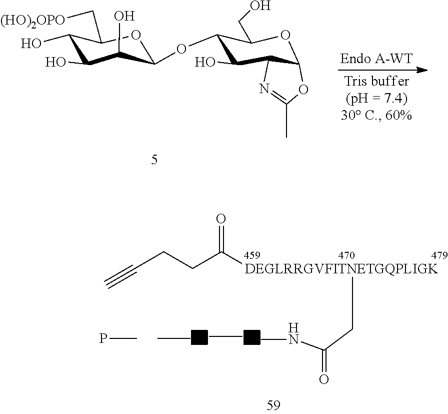

GlcNAc-peptide (3.0 mg, 1.16 μmol) was incubated at 30° C. together with oxazoline 5 (2.1 mg, 4 eq) and Endo A-WT (120 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 59 (2.1 mg, 60%) as white solid. ESI-MS: Calcd., M=3029.20; found (m/z): 758.13 [M+4H]⁴⁺, 1010.14 [M+3H]³⁺, 1515.15 [M+2H]²⁺. Deconvolution of the ESI-MS: M=3028.7; RP-HPLC retention time, t_{R}=19.5 min (gradient, 5-40% aq MeCN containing 0.1% FA for 30 min; flow rate, 1.0 mL/min).

### Glycopeptide 60

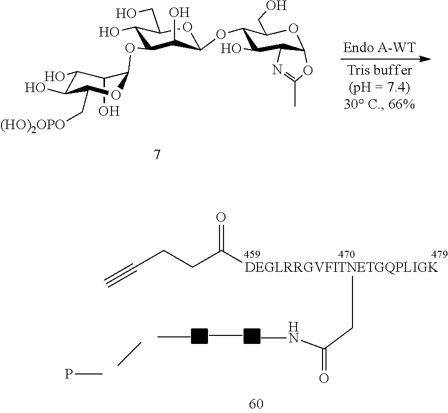

GlcNAc-peptide (3.0 mg, 1.16 μmol) was incubated at 30° C. together with oxazoline 7 (3.5 mg, 5 eq) and Endo A-WT (120 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 60 (2.4 mg, 66%) as white solid. ESI-MS: Calcd., M=3191.34; found (m/z): 798.74 [M+4H]⁴⁺, 1064.61 [M+3H]³⁺, 1596.46 [M+2H]²⁺. Deconvolution of the ESI-MS: M=3190.9; RP-HPLC retention time, t_{R}=19.4 min (gradient, 5-40% aq MeCN containing 0.1% FA for 30 min; flow rate, 1.0 mL/min).

### Glycopeptide 61

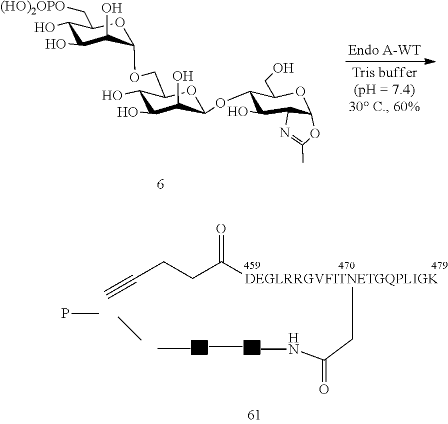

GlcNAc-peptide (3.0 mg, 1.16 μmol) was incubated at 30° C. together with oxazoline 6 (2.8 mg, 4 eq) and Endo A-WT (60 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 61 (2.2 mg, 60%) as white solid. ESI-MS: Calcd., M=3191.34; found (m/z): 798.74 [M+4H]⁴⁺, 1064.86 [M+3H]³⁺, 1596.58 [M+2H]²⁺. Deconvolution of the ESI-MS: M=3190.4; RP-HPLC retention time, t_{R}=19.2 min (gradient, 5-40% aq MeCN containing 0.1% FA for 30 min; flow rate, 1.0 mL/min).

### Glycopeptide 62

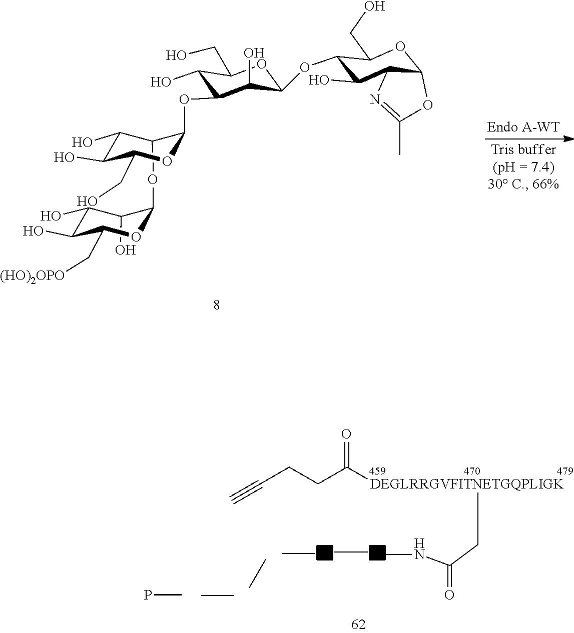

GlcNAc-peptide (3.0 mg, 1.16 μmol) was incubated at 30° C. together with oxazoline 8 (4.2 mg, 5 eq) and Endo A-WT (60 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 62 (2.6 mg, 66%) as white solid. ESI-MS: Calcd., M=3353.48; found (m/z): 839.27 [M+4H]⁴⁺, 1118.50 [M+3H]³⁺, 1677.12 [M+2H]²⁺. Deconvolution of the ESI-MS: M=3352.7; RP-HPLC retention time, t_{R}=19.2 min (gradient, 5-40% aq MeCN containing 0.1% FA for 30 min; flow rate, 1.0 mL/min).

### Glycopeptide 63

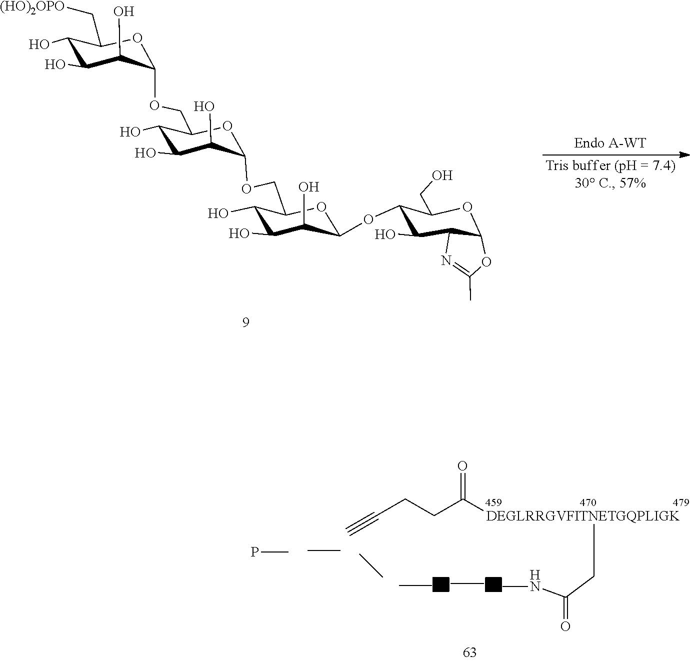

GlcNAc-peptide (3.0 mg, 1.16 μmol) was incubated at 30° C. together with oxazoline 9 (4.8 mg, 6 eq) and Endo A-WT (120 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 63 (2.2 mg, 57%) as white solid. ESI-MS: Calcd., M=3353.48; found (m/z): 839.25 [M+4H]⁴⁺, 1118.85 [M+3H]³⁺, 1677.48 [M+2H]²⁺. Deconvolution of the ESI-MS: M=3353.1; RP-HPLC retention time, t_{R}=19.2 min (gradient, 5-40% aq MeCN containing 0.1% FA for 30 min; flow rate, 1.0 mL/min).

### Glycopeptide 64

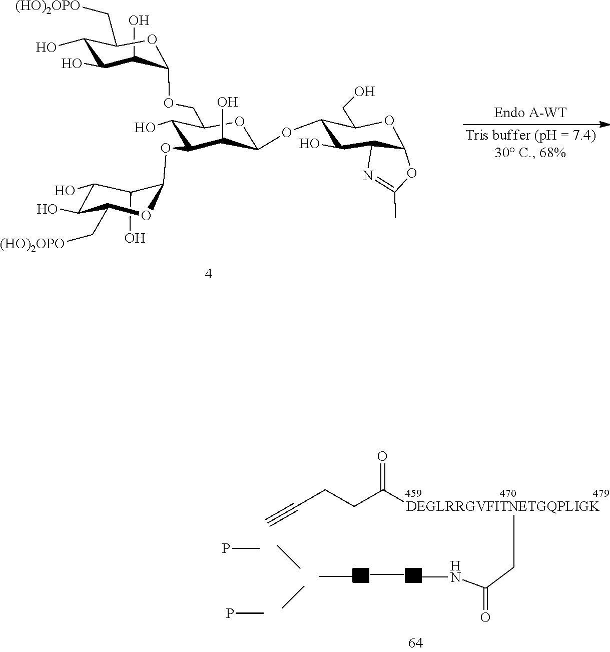

GlcNAc-peptide (3.0 mg, 1.16 μmol) was incubated at 30° C. together with oxazoline 4^{[7]} (4.9 mg, 5 eq) and Endo A-WT (100 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 64 (2.7 mg, 68%) as white solid. ESI-MS: Calcd., M=3433.46; found (m/z): 859.25 [M+4H]⁴⁺, 1145.02 [M+3H]³⁺, 1717.00 [M+2H]²⁺. Deconvolution of the ESI-MS: M=3432.9; RP-HPLC retention time, t_{R}=20.2 min (gradient, 5-40% aq MeCN containing 0.1% FA for 30 min; flow rate, 1.0 mL/min).

### Glycopeptide 65

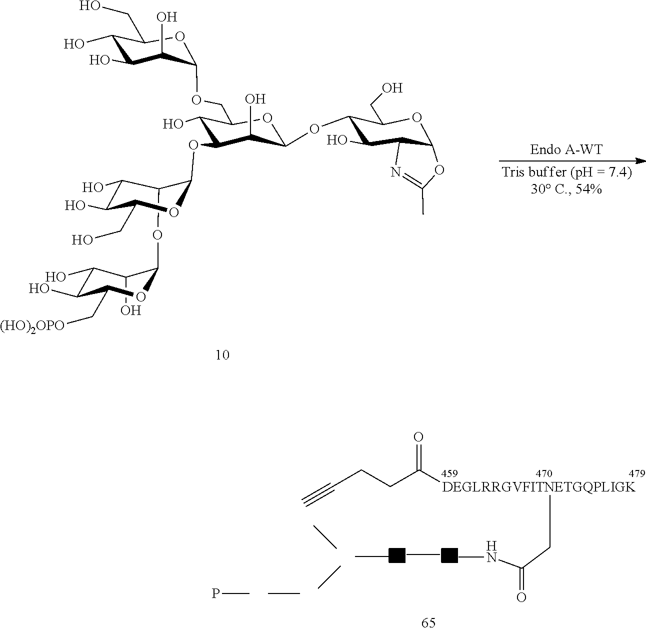

GlcNAc-peptide (3.0 mg, 1.16 μmol) was incubated at 30° C. together with oxazoline 10 (5.4 mg, 5 eq) and Endo A-WT (120 μg) in Tris buffer (100 mM, pH 7.4, 100 μL). The reaction was monitored by analytical RP-HPLC. Upon completion of the transglycosylation, the reaction was quenched using 0.1% aq. TFA and purified by RP-HPLC (gradient, 10-40% aq MeCN containing 0.1% FA for 30 min; flow rate, 4.0 mL/min) to give glycopeptides 65 (2.2 mg, 54%) as white solid. ESI-MS: Calcd., M=3515.62; found (m/z): 879.89 [M+4H]⁴⁺, 1172.78 [M+3H]³⁺, 1758.44 [M+2H]²⁺. Deconvolution of the ESI-MS: M=3515.6; RP-HPLC retention time, t_{R}=19.1 min (gradient, 5-40% aq MeCN containing 0.1% FA for 30 min; flow rate, 1.0 mL/min).

### Glycopeptide 66

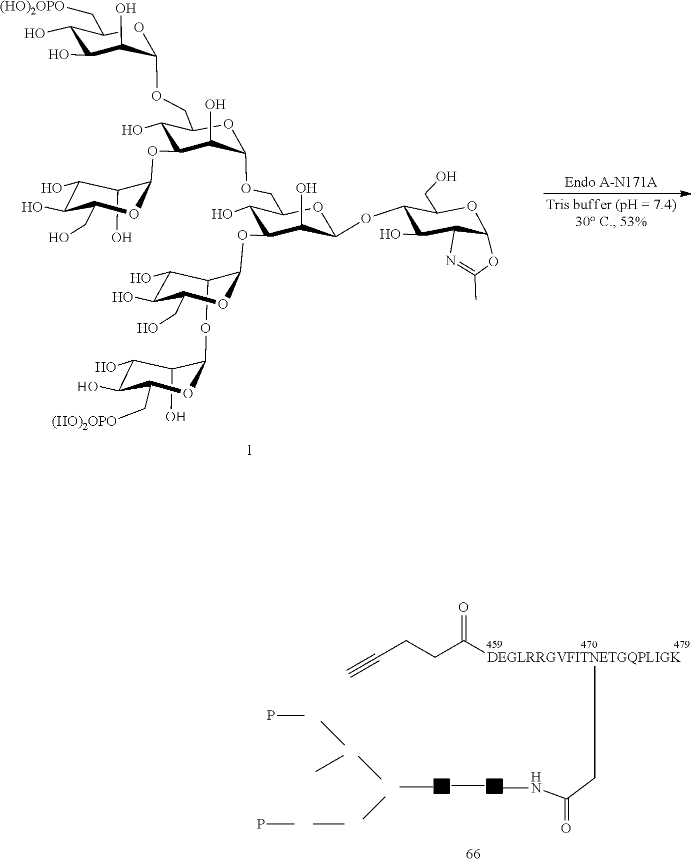

GlcNAc-peptide (2.0 mg, 0.77 μmol) was incubated at 30° C. together with phosphorylated oxazoline 1^{[7]} (5.2 mg, 5 eq) and Endo A-N171A (180 μg) in Tris buffer (100 mM, pH 7.4, 100 μl). The reaction was monitored by analytical RP-HPLC. Upon completion of the transglycosylation, the reaction was quenched using 0.1% aq. TFA and purified by RP-HPLC to give glycopeptides 66 (1.54 mg, 53%). ESI-MS: Calcd., M=3919.88; found (m/z): 980.88 [M+4H]⁴⁺, 1307.60 [M+3H]³⁺, 1960.92 [M+2H]²⁺. Deconvolution of the ESI-MS: M=3919.5; RP-HPLC retention time, t_{R}=19.9 min (gradient, 5-40% aq MeCN containing 0.1% FA for 30 min; flow rate, 1.0 mL/min).

### Example 11: Glycan Remodeling

### Glycoprotein 68

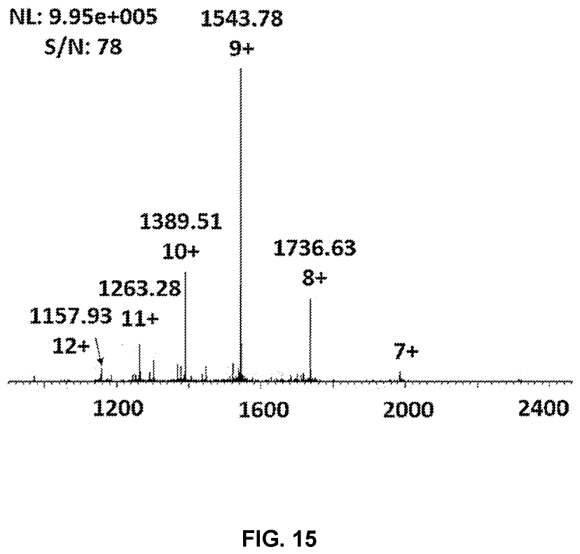

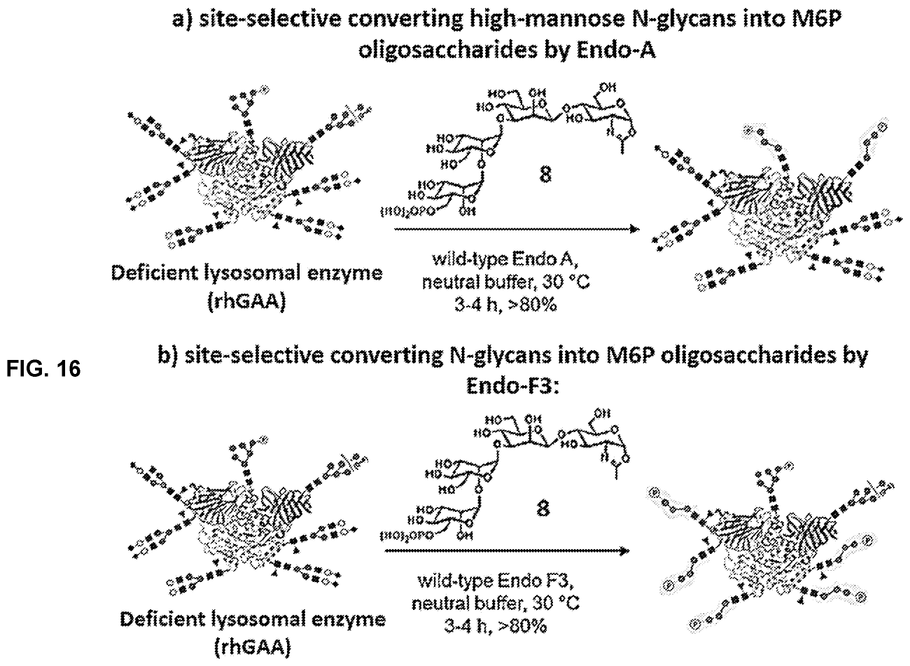

Stepwise strategy. RNase B (5.8 mg) was treated with wild-type Endo A (66 μg) in PBS buffer (pH=7.2, 580 μL) at 37° C. for 1 h. Upon the completion of the reaction as monitored by analytical RP-HPLC, the reaction was purified by preparative HPLC to give the homogeneous GlcNAc-RNase B 67 (4.6 mg, 86%). ESI-MS: Calcd., M=13886; found (m/z): 1157.93 [M+12H]¹²⁺, 1263.28 [M+11H]¹¹⁺, 1389.51 [M+10H]¹⁰⁺, 1543.78 [M+9H]⁹⁺, 1736.63 [M+8H]⁸⁺. Deconvolution of the ESI-MS: M=13886 (FIG. 15).

Then to a solution of oxazoline 8 (500 μg, 0.65 μmol, 9 eq) and GlcNAc-RNase B 67 (1.0 mg, 0.072 μmol) in PBS buffer (150 mM, pH 7.2, 20 μL) was added Endo A-WT (200 μg) at 30° C. The reaction was monitored with analytical RP-HPLC. After 3 h, another portion of oxazoline (280 μg, 5 eq) was added and this procedure was repeated 2 to 3 times until the GlcNAc-RNase B was consumed. Upon completion of the transglycosylation, the reaction was purified by RP-HPLC to give glycoprotein 68 as white solid (0.74 mg, 71%). ESI-MS: Calcd., M=14655; found (m/z): 1047.87 [M+14H]¹⁴⁺, 1128.39 [M+13H]¹³⁺, 1222.13 [M+12H]¹²⁺, 1333.33 [M+11H]¹¹⁺, 1466.57 [M+10H]¹⁰⁺, 1629.37 [M+9H]⁹⁺, 1832.80 [M+8H]⁸⁺. Deconvolution of the ESI-MS: M=14656. RP-HPLC retention time, t_{R}=17.4 min (gradient, 5-40% aq MeCN containing 0.1% FA for 30 min; flow rate, 1.0 mL/min). In this step, 0.93 mg of solid was obtained after HPLC purification, and ca.˜ 80% was the desired glycoprotein 68 according to the MS spectrum, which was not separable from the starting material due to the small size of the tetrasaccharide, so the yield was calculated as follows: 930 μg*80%=744 μg (0.051 μmol), 0.051/0.072=70.5%. The same method was used in the “one-pot” strategy.

“One-pot” strategy. RNase B (1.0 mg) was incubated with Endo A-WT (200 μg) in PBS buffer (pH=7.2, 20 μL) at 30° C. for 30 min before the addition of oxazoline 8 (308 μg, 6 eq). The reaction was monitored with analytical RP-HPLC, and additional oxazoline was added to push the reaction to completion as described in the stepwise strategy. Preparative RP-HPLC afforded the desired glycoprotein 68 as white solid (0.62 mg, 64%).

“One-Pot” Glycan Remodeling of rhGAA with Wild-Type Endo A

The commercial Lumizyme (Genzyme, Sanofi) was purified by buffer exchange with PBS (150 mM, pH=7.2) to remove extra additions before use. The resulting rhGAA (0.95 mg) was incubated with Endo A-WT (100 μg) in PBS buffer (pH=7.2, 25 μL) at 30° C. for 2 h before the addition of oxazoline 8 (125 μg, 15 eq). After 30 min, another batch of oxazoline (125 μg, 15 eq) was added and this procedure was repeated 6 to 7 times to consume most of the starting material. Upon the completion of the reaction, the resulting mixture was treated with Glutathione Agarose (Thermo Fisher, resin suspended in 200 μL solution) to remove the GST-tagged Endo A-WT, and the cleaved glycans and extra salts were removed by buffer exchange (PBS×5) to get the remodeled rhGAA (820 μg, 86%).

Transglycosylation of α1,6FucGlcNAc-CD52 with Wild-Type Endo F3

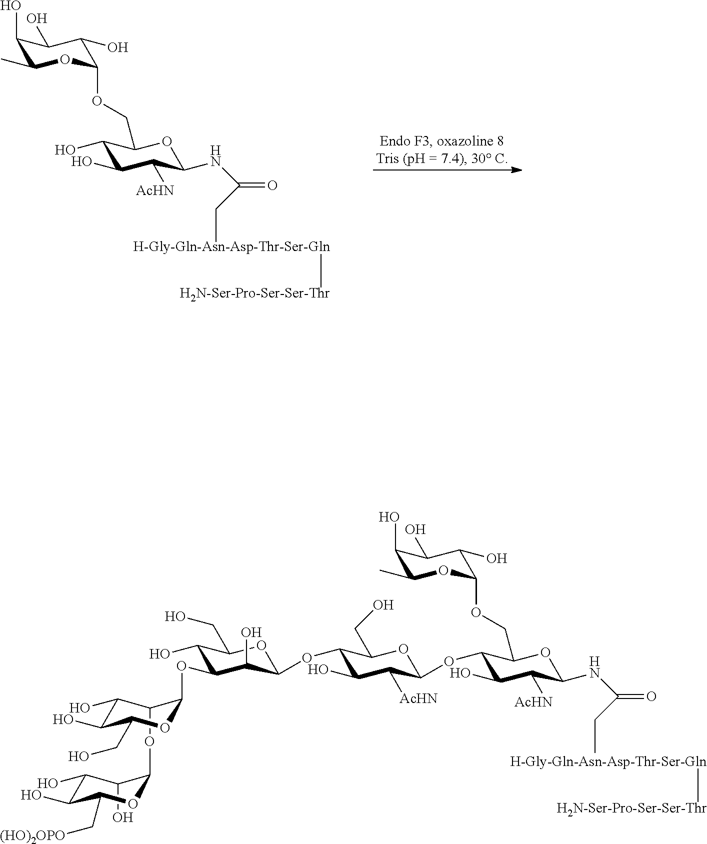

To a solution of oxazoline 8 (200 μg, 0.26 μmol, 4 eq) and Fuca1,6GlcNAc-CD52 (100 μg, 0.072 μmol) in Tris buffer (100 mM, pH 7.4, 5 μL) was added Endo F3-WT (3.0 μg) at 30° C. The reaction was complete within 30 min. MALDI-TOF: [M+H]⁺ calcd for C₈₅H₁₄₂N₁₈O₅₅P⁺, 2327.12; found, 2327.65; [M+Na]⁺ calcd for C₈₅H₁₄₁N₁₈NaO₅₅P⁺, 2349.10; found, 2349.51; [M−H+2Na]⁺ calcd for C₈₅H₁₄₀N₁₈Na₂O₅₅P⁺, 2371.08; found, 2371.50; [M−2H+3Na]⁺ calcd for C₈₅H₁₃₉N₁₈Na₃₀₅₅PT, 2393.06; found, 2393.49; [M−H]⁻ calcd for C₈₅H₁₄₀N₁₈O₅₅P⁺, 2325.10; found, 2325.84; [M−2H+Na]⁻ calcd for C₈₅H₁₃₉N₁₈NaO₅₅P⁺, 2347.08; found, 2348.04.

“One-Pot” Glycan Remodeling of rhGAA with Wild-Type Endo F3

The commercial Lumizyme (Genzyme, Sanofi) was directly used without pretreatment (the additions such as mannitol were necessary to keep the protein soluble because Endo F3 would cleave most of the complex-type N-glycans). To the commercial mixture (2.4 mg powder, containing ˜400 μg rhGAA) in PBS (pH=7.2, 10 μL) was added Endo F3-WT (40 μg) and oxazoline 8 (100 μg, 30 eq). After 2 h, another batch of oxazoline (100 μg, 30 eq) was added and this procedure was repeated twice to consume most of the starting material. Upon the completion of the reaction, the resulting mixture was treated with Histrap column (GE Healthcare, 1 mL) to remove the His-tagged Endo F3-WT, and the cleaved glycans and extra salts were removed by buffer exchange (PBS×5) to get the remodeled rhGAA (282 μg, 71%).

### Example 12: Biological Studies

Surface Plasmon Resonance (SPR) Measurements. SPR measurements were performed on a Biacore T200 instrument (GE Healthcare). Recombinant human IGF-II R (CI-MPR) was purchased from R&D Systems. Approximately 7000 resonance units (RU) of CI-MPR was immobilized on a CM5 sensor chip in a sodium acetate buffer (25 μg/mL, pH 4.0) at 25° C., using the amine coupling kit provided by the manufacturer. Mannose 6-phosphates containing glycopeptides or glycoproteins were determined at 25° C. under a flow rate of 10 μL/min. HBS-P+ buffer (10 mM HEPES, 150 mM NaCl, 0.05% surfactant P20, pH 7.4) was used as sample buffer and running buffer. Association was measured for 3 min and dissociation for 10 min at the same flow rate (10 μL/min). The surface regeneration was performed by 2 M MgCl₂ at a flow rate of 10 μL/min for 60 s. Synthetic glycopeptide and glycoprotein analytes flowed over an immobilized chip with 2-fold serial dilution of the highest concentration of 4 μM (for glycopeptides) or 1 μM (for RNase B) or 250 nM (for rhGAA). Kinetic analyses were performed by global fitting of the binding data to a 1:1 Langmuir binding model using BIAcore T200 evaluation software.

rhGAA enzyme activity assay. The enzyme activity was assayed by using the substrate 4-methylumbelliferyl-α-D-glucopyranoside (4-MUG) (Sigma-Aldrich) which generates fluorescence on digestion.^{[9, 10]} To a solution of 4-MUG (3.0 mM) in acetate buffer (200 μL, containing 0.2 M sodium acetate, 0.4 M potassium chloride, pH 4.3) was added 1.0 μg of rhGAA or remodeled rhGAA (˜ 50 nM), and the reaction mixture was incubated at 37° C. The reaction was monitored at 0 min, 1 min, 2 min, 5 min, 10 min and 15 min by taking 20 μL of aliquot and adding 50 μL of stop buffer (100 mM glycine/NaOH, pH 11). Fluorescence was measured by a spectrophotometer with 355 nm excitation and 460 nm emission, and the error bar was based on three independent assays.

Muscle cell culture, treatment, processing, and analysis. The biological effect of the Endo-A (69) and Endo-F3 (70) remodeled rhGAA was investigated in an in vitro model of Pompe disease.^{[11, 12]} The myoblasts are grown on Matrigel (Corning; 354234)-coated 6-well plates at 33° C. in an atmosphere of 5% CO₂ in proliferation medium [20% fetal bovine serum, 10% horse serum, 1% chick embryo extract, recombinant IFN-γ (100 U/mL; Life Technologies), 1× penicillin/streptomycin/L-glutamine in high-glucose (4.5 g/L) DMEM]. When the cells reach 70-80% confluency (3-4 days), the medium is switched to differentiation medium [DMEM containing 2% horse serum, 0.5% chick embryo extract, recombinant human insulin (10 μg/mL, Life Technologies, 12585-014), 1× penicillin/streptomycin/L-glutamine], and the cells are moved to 37° C. in an atmosphere of 5% CO₂. Myotubes begin to form within 3-4 days; they can survive for ˜ 8-10 days in culture until they start twitching and detach from the surface.

The commercial rhGAA, Endo-A- or Endo-F3 remodeled rhGAA were added to the myotubes (on day 8 in differentiation medium) at a concentration of 5 μM for 24 hours; n=5 independent experiments for each condition. Wild type (WT) immortalized myotubes and untreated KO myotubes were used for comparison. The cells were homogenized on ice in deionized H₂O, sonicated, and centrifuged at 18,000×g at 4° ° C. for 15 min. The supernatants were used for measuring GAA activity and glycogen content.

Measurement of cell-associated GAA activity. The GAA activity in the cells was measured by using 4-methylumbelliferyl-α-D-glucopyranoside (4-MU-α-glucopyranoside; Sigma-Aldrich #M9766) as the fluorogenic GAA substrate as described.^{[13]} Briefly, myotubes grown on Matrigel-coated 6-well plates were rinsed 3 times with PBS, homogenized in distilled water (using a syringe-based homogenization), sonicated, and centrifuged at 18,000×g at 4° C. for 15 min. The supernatants were incubated with the substrate in 0.2 M sodium acetate buffer (pH 4.3) in 96-well plates for 1 h at 37° C.; the reaction was stopped by adding 0.5 M carbonate buffer (pH 10.5). 4-Methylumbelliferone (4-MU; Sigma-Aldrich #M1381) was used as a standard. Fluorescence was measured on a multi-label plate reader (TECAN, SPARK 10M) at 360 nm excitation/465 nm emission.

Measurement of the glycogen content. The glycogen content was measured as the amount of glucose released after glycogen digestion with Aspergillus Niger amyloglucosidase (Sigma-Aldrich). Samples were denatured at 100° C. for 3 min to inactivate endogenous enzymes, centrifuged at 9,000 RPM at room temperature for 3 min, and the supernatants were incubated with/or without 0.175 U/mL amyloglucosidase for 90 min at 37° C. in 0.1 M potassium acetate buffer (pH 5.5) and boiled again to stop the reaction. The released glucose was measured using Glucose (Hexokinase) Liquid Reagents (Fisher) as recommended by the manufacturer; the absorbance at 340 nm was read on the Agilent Technologies Cary 60 UV-VIS Spectrophotometer. Protein concentration (BCA assay) was measured and used to normalize the data.

Western blotting. The myotubes were extensively washed, homogenized in RIPA buffer (PBS containing 1% NP-40, 0.5% sodium deoxycholate, 0.1% SDS, and a protease/phosphatase inhibitor cocktail), and centrifuged for 10 min at 18,000×g at 4° C. Protein concentrations of the supernatants were measured using the Bio-Rad Protein Assay (Bio-Rad Laboratories, Inc.), and equal amounts of protein were run on SDS-PAGE gels (Invitrogen, Carlsbad, CA). Separated proteins were electro-transferred onto nitrocellulose membranes (Invitrogen, Carlsbad, CA, USA). Membranes were then treated with blocking buffer (5% nonfat milk), incubated with primary antibodies [rat monoclonal anti-mouse LAMP-1 (Lysosomal-Associated Membrane Protein 1; CD107a #553792) and mouse monoclonal anti-Rab5 (#610724) from BD Pharmingen; rabbit monoclonal anti-human GAA(EPR4716(2) from Abcam] overnight at 4ºC, washed, incubated with the appropriate Alexa Fluor-conjugated secondary antibodies and washed again. Horseradish peroxidase (HRP)-chemiluminescence was developed using Azure Radiance plus kit and scanned on imager (Azure Biosystems). Mouse monoclonal anti-GAPDH antibody (Abcam, ab9484) served as loading controls.

For immunofluorescence, cultured myotubes were fixed with 2% paraformaldehyde (PFA) for 30 min at room temperature, followed by several washes with PBS and incubation with blocking reagent (MOM kit; Vector Laboratories, Burlingame, CA) for 1 h at room temperature. Myotubes were then incubated with primary antibodies overnight at 4° C., washed with PBS, incubated with secondary antibodies for 2 h, and washed again before examination by confocal microscopy (Zeiss LSM 880).

Statistical significance was determined by one way ANOVA testing using Prism software. Error bars represent SD. * P<0.05 was considered statistically significant. ** indicate P-values<0.01; *** indicate P-values<0.001.

Each publication, patent, and patent publication cited in this disclosure is incorporated in reference herein in its entirety. The present disclosure is not intended to be limited only to the foregoing examples, but encompasses all such modifications and variations as come within the scope of the appended claims.

1. A compound of Formula (I), or a salt thereof, 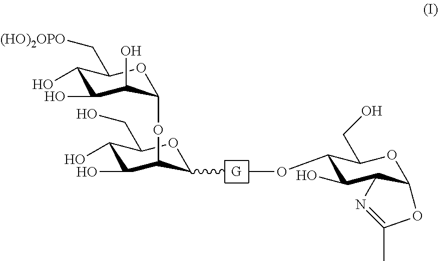
wherein G is sugar moiety or linker.

2. (canceled)

3. The compound of claim 1, having a structure of Formula (I-a), or a salt thereof

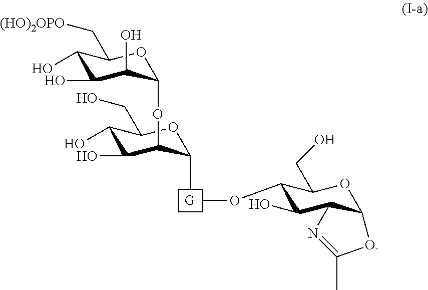

4. The compound of claim 1, or a salt thereof, wherein G is a sugar moiety, optionally wherein G is a monosaccharide moiety, a disaccharide moiety, a trisaccharide moiety, or a tetrasaccharide moiety.

5. (canceled)

6. (canceled)

7. (canceled)

8. A method for remodeling a glycoprotein, comprising:
(a) contacting the glycoprotein with an endoglycosidase selected from the group consisting of wild type Endo A, wild type Endo F3, wild type Endo-CC, and a combination of, thereby producing a deglycosylated intermediate comprising a N-acetylglucosamine (GlcNAc) or core-fucosylated N-acetylglucosamine (Fuca1,6GlcNAc) acceptor from the glycoprotein by a deglycosylation activity of the endoglycosidase to produce a deglycosylated intermediate; and
(b) contacting a glycan oxazoline comprising a mannose-6-phosphate (M6P) moiety with the deglycosylated intermediate in the presence of the endoglycosidase, thereby attaching the glycan oxazoline to the N-acetylglucosamine (GlcNAc) or core-fucosylated N-acetylglucosamine (Fuca1,6GlcNAc) acceptor by a transglycosylation activity of the endoglycosidase, thereby producing a remodeled glycoprotein,
wherein (a) and (b) are carried out in a one-pot reaction.

9. The method of claim 8, wherein the glycoprotein is a lysosomal enzyme.

10. The method of claim 8, wherein the endoglycosidase is wild type Endo A, optionally wherein the wild type Endo A removes high-mannose and hybrid type glycans from the lysosomal enzyme without affecting complex-type glycans.

11. The method of claim 8, wherein the endoglycosidase is wild type Endo F3, optionally wherein the wild type Endo F3 removes core-fucosylated complex-type glycans from the lysosomal enzyme without affecting high-mannose or hybrid type glycans.

12. The method of claim 8, wherein the endoglycosidase is wild type Endo-CC, optionally wherein the wild type Endo-CC removes high-mannose type and biantennary complex type glycans from the lysosomal enzyme without affecting core-fucosylated complex-type glycans or higher branched complex type glycans.

13. The method of claim 8, wherein the endoglycosidase is a combination of the wild type Endo A and the wild type Endo F3.

14. The method of claim 8, wherein the lysosomal enzyme is selected from the group consisting of α-galactosidase A, acid ceramidase, acid α-L-fucosidase, acid β-glucosidase, acid β-galactosidase, iduronate-2-sulfatase, α-L-iduronidase, galactocerebrosidase, acid α-mannosidase, acid β-mannosidase, arylsulfatase B, arylsulfatase A, N-acetylgalactosamine-6-sulfate sulfatase (N-acetylgalactosamine-6-sulfatase, or galactose-6-sulfatase), acid β-galactosidase, acid sphingomyelinase, acid α-glucosidase (α-glucosidase), β-hexosaminidase B, heparan N-sulfatase, α-N-acetylglucosaminidase, acetyl-CoA: α-glucosaminide N-acetyltransferase, N-acetylglucosaminide-6-sulfate sulfatase, α-N-acetylgalactosaminidase, sialidase, β-glucuronidase, β-hexosaminidase A, and a combination thereof, optionally wherein the lysosomal enzyme comprises at least one asparagine (N)-linked glycan.

15. The method of claim 8, wherein the glycan oxazoline comprises at least one Man6Pa1,2Man moiety,

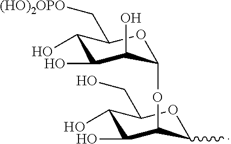

16. The method of claim 8, wherein the glycan oxazoline is a disaccharide oxazoline, trisaccharide oxazoline, tetrasaccharide oxazoline or pentasaccharide oxazoline.

17. The method of claim 8, wherein the glycan oxazoline has a structure of formula (I), or a salt thereof, 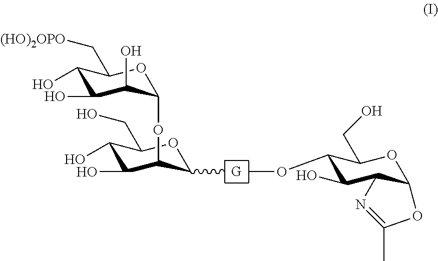
wherein G is bond or a linker, optionally wherein the linker is a sugar moiety.

18. The method of claim 8, wherein the glycan oxazoline is selected from the group consisting of 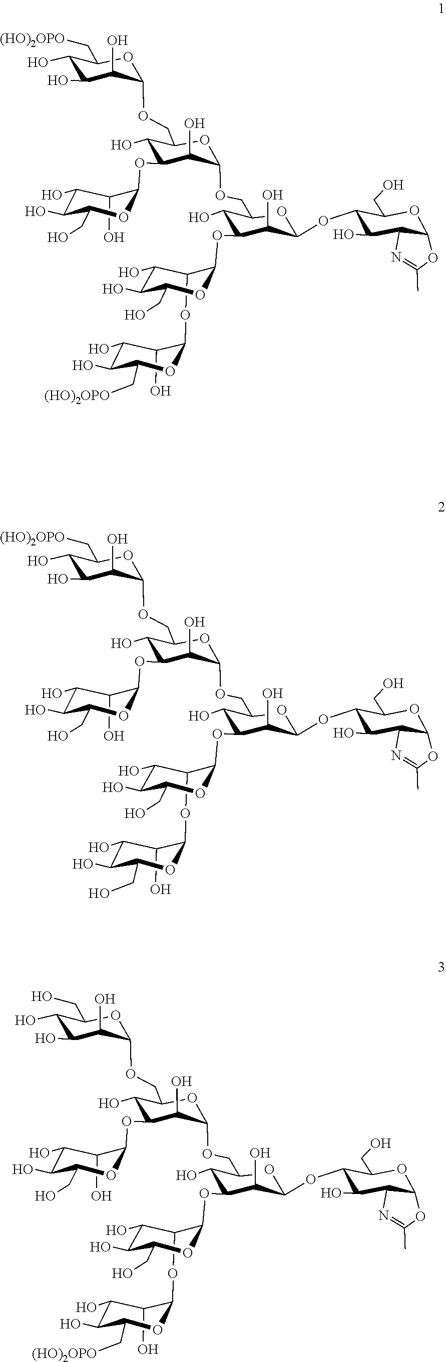 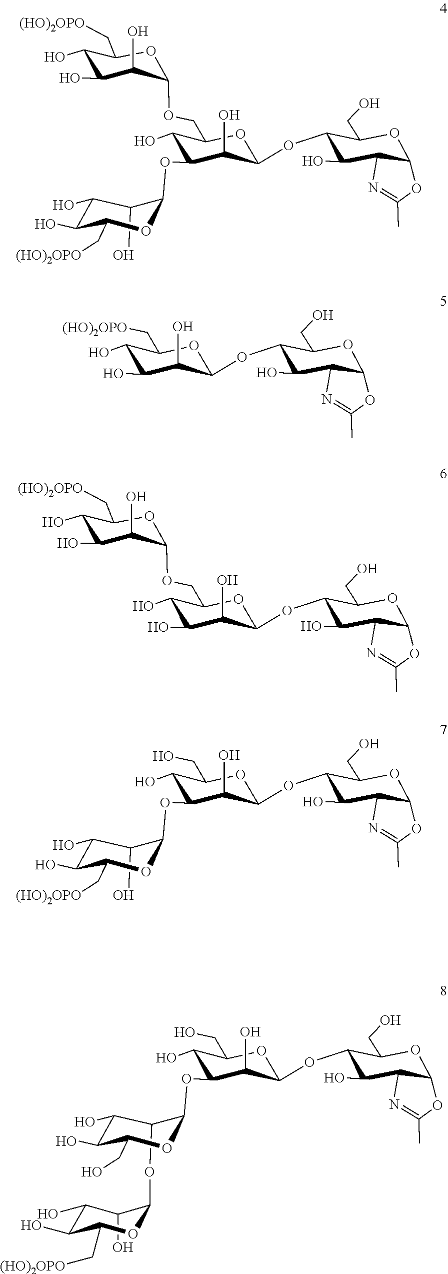 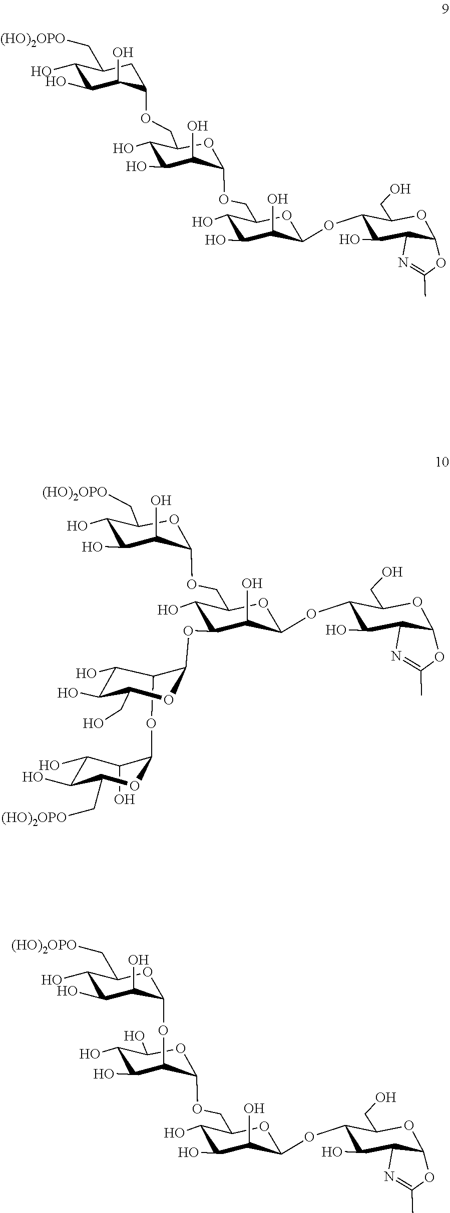 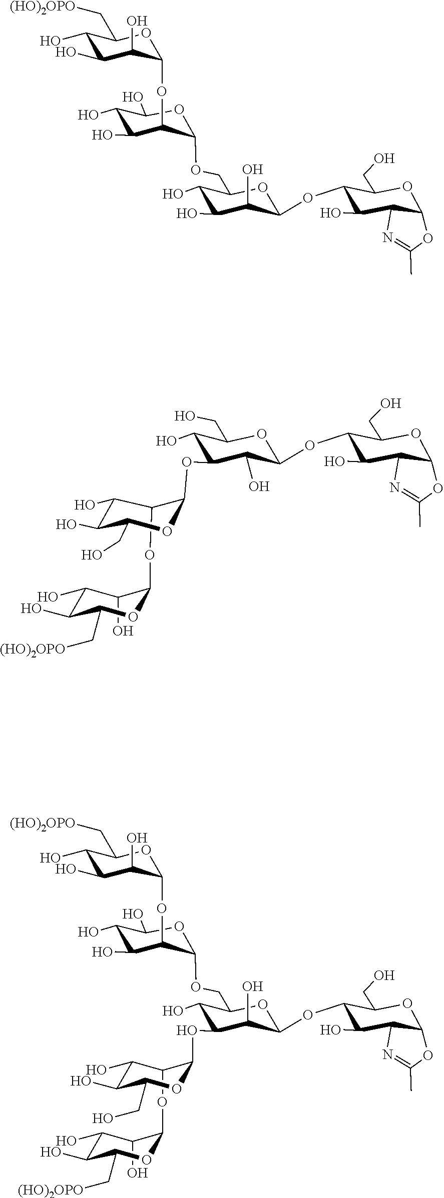 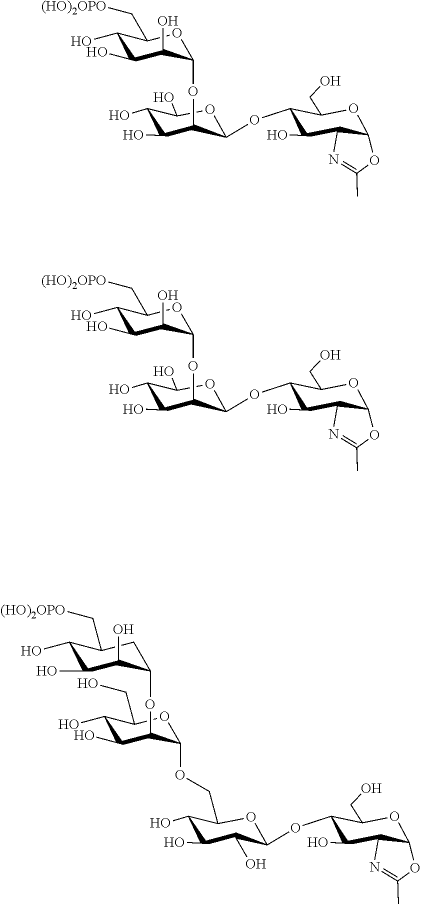
or a salt thereof.

19. The method of claim 8, wherein the glycan 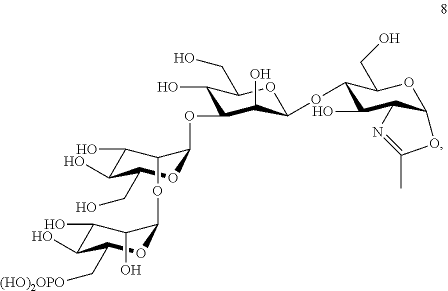
oxazoline is or a salt thereof.

20. A method of enhancing binding affinity of a glycoprotein to a cation-independent M6P receptor (CI-MPR), comprising remodeling the glycoprotein according to the method of claim 8 and contacting the glycoprotein with a cell comprising CI-MPR receptor, thereby enhancing binding affinity of the glycoprotein to the CI-MPR.

21. A method of enhancing or increasing uptake of a glycoprotein in a cell, comprising
(a) remodeling the glycoprotein according to the method of claim 8, and
(b) contacting the cell with the remodeled glycoprotein, thereby enhancing uptake of the glycoprotein in the cell.

22. (canceled)

23. (canceled)

24. The method of claim 21, wherein the glycoprotein is acid α-glucosidase (α-glucosidase), and the cell is a muscle cell.

25. A glycan-remodeled glycoprotein produced by the method of claim 8.

26. (canceled)

27. (canceled)

28. A method of treating Pompe disease in a subject in need thereof, comprising administering to the subject a pharmaceutically effective amount of the glycan-remodeled lysosomal enzyme of claim 24.

29. (canceled)

30. (canceled)


SEQUENCE LISTING (27 sequences)

SEQ ID NO: 1 - protein, 621 aa, Arthrobacter protophormiae
STYNGPLSSHWFPEELAQWEPDSDPDAPFNRSHVPLEPGRVANRVNANADKDAHLVSLSALNRHTSGVPSQGAPVFYENTFSYWHYTDLMVYWAGSAGEGIIVPPSADVIDASHRNGVPILGNVFFPPTVYGGQLEWLEQMLEQEEDGSFPLADKLLEVADYYGFDGWFINQETEGADEGTAEAMQAFLVYLQEQKPEGMHIMWYDSMIDTGAIAWQNHLTDRNKMYLQNGSTRVADSMFLNFWWRDQRQSNELAQALGRSPYDLYAGVDVEARGTSTPVQWEGLFPEGEKAHTSLGLYRPDWAFQSSETMEAFYEKELQFWVGSTGNPAETDGQSNWPGMAHWFPAKSTATSVPFVTHFNTGSGAQFSAEGKTVSEQEWNNRSLQDVLPTWRWIQHGGDLEATFSWEEAFEGGSSLQWHGSLAEGEHAQIELYQTELPISEGTSLTWTFKSEHGNDLNVGFRLDGEEDFRYVEGEQRESINGWTQWTLPLDAFAGQTITGLAFAAEGNETGLAEFYIGQLAVGADSEKPAAPNVNVRQYDPDPSGIQLVWEKQSNVHHYRVYKETKHGKELIGTSAGDRIYLEGLVEESKQNDVRLHIEALSETFVPSDARMIDIKSGSF

SEQ ID NO: 2 - protein, 290 aa, Elizabethkingia meningoseptica
TALAGSNGVCIAYYITDGRNAPTFKLKDIPDKVDMVILFGLKYWSLQDTTKLPGGTGMMGSFKSYKDLDTQIRSLQSRGIKVLQNIDDDVSWQSSKPGGFASAAAYGDAIKSIVIDKWKLDGISLDIEHSGAKPNPIPTFPGYAATGYNGWYSGSMAATPAFLNVISELTKYFGTTAPNNKQLQIASGIDVYAWNKIMENFRNNFNYIQLQSYGANVSRTQLMMNYATGTNKIPASKMVFGAYAEGGTNQANDVEVAKWTPTQGAKGGMMIYTYNSNVSYANAVRDAVKN

SEQ ID NO: 3 - protein, 787 aa, Coprinus cinereus
MPIAGKKFHPRALPEFWRTFREMDEWRATQTGPQARPAEGILKYVPRKIRPADIAGKGRLLVSHDYKGGYVEDPFSKSYSFNWWFSTDSFNYFAHHRITIPPPEWINAAHRQGVPILGTIIFEGGSDEDILRMVIGKTPGSTSNFHAERNAEYTVPVSSYYAELFADLAVERGFDGWLLNVEIGLQGGSEQARGLAAWVALLQQEVLKKVGPHGLVIWYDSVTVRGDLWWQDRLNAFNLPFFLNSSGIFTNYWWYNDAPQKQIDFLSRVDPNLTGQTAEPHQYNLQKTIQDIYIGVDVWGRGSHGGGGFGAYKAIEHADPKGLGFSVALFAQGWTWETEEEKPGWNWAQFWDYDSKLWVGPPGVVEAPDHTVKPGEYPCVHGPFQPISSFFLTYPPPDPLDLPFYTNFCPGIGDAWFVEGKEVFRSETGWTDMDKQTTVGDLVWPRPKIYDLPSQNASQATLNAAFNFNDAWNGGNSLQINLTVPGGATTYGAYWVPIQTFTFSSRRQYEASIVYKPGLSGKTRFDAKYEVGIRTITGEDQGKIISNTTTEVGNGWRKVHILFEIETPVEGGSIIVPSSIGLVIAVSNVSTTEQFEFPFLVGQITIHPHLPDRYKEFKPALLWLLFTPSAGTNSLDGTLTWDVVAAIERPPPVEINNPDDAQIPWNLQPTKQEWFPDFLYFNVYVLELLDGGGQGPPQWIGTTGYDGEKKRFFIYDESLPPTSGLRRFTFQIEGVLETGESTHWYDAPAAPSATAGGEQKRTRRTSLKSVLSPLRRKKSKGDISVAK

SEQ ID NO: 4 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 5 - protein, 395 aa, Homo sapiens
MPGRSCVALVLLAAAVSCAVAQHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 6 - protein, 466 aa, Homo sapiens
MRAPGMRSRPAGPALLLLLLFLGAAESVRRAQPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 7 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 8 - protein, 677 aa, Homo sapiens
MPGFLVRILPLLLVLLLLGPTRGLRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 9 - protein, 550 aa, Homo sapiens
MPPPRTGRGLLWLGLVLSSVCVALGSETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 10 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 11 - protein, 685 aa, Homo sapiens
MAEWLLSASWQRRAKAMTAAAGSAGRAAVPLLLCALLAPGGAYVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFTSGMLNDKSLWTDIPVNFPKNGWAAIGTHSFEFAQFDNFLVEATR

SEQ ID NO: 12 - protein, 1011 aa, Homo sapiens
MGAYARASGVCARGCLDSAGPWTMSRALRPPLPPLCFFLLLLAAAGARAGGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG

SEQ ID NO: 13 - protein, 879 aa, Homo sapiens
MRLHLLLLLALCGAGTTAAELSYSLRGNWSICNGNGSLELPGAVPGCVHSALFQQGLIQDSYYRFNDLNYRWVSLDNWTYSKEFKIPFEISKWQKVNLILEGVDTVSKILFNEVTIGETDNMFNRYSFDITNVVRDVNSIELRFQSAVLYAAQQSKAHTRYQVPPDCPPLVQKGECHVNFVRKEQCSFSWDWGPSFPTQGIWKDVRIEAYNICHLNYFTFSPIYDKSAQEWNLEIESTFDVVSSKPVGGQVIVAIPKLQTQQTYSIELQPGKRIVELFVNISKNITVETWWPHGHGNQTGYNMTVLFELDGGLNIEKSAKVYFRTVELIEEPIKGSPGLSFYFKINGFPIFLKGSNWIPADSFQDRVTSELLRLLLQSVVDANMNTLRVWGGGIYEQDEFYELCDELGIMVWQDFMFACALYPTDQGFLDSVTAEVAYQIKRLKSHPSIIIWSGNNENEEALMMNWYHISFTDRPIYIKDYVTLYVKNIRELVLAGDKSRPFITSSPTNGAETVAEAWVSQNPNSNYFGDVHFYDYISDCWNWKVFPKARFASEYGYQSWPSFSTLEKVSSTEDWSFNSKFSLHRQHHEGGNKQMLYQAGLHFKLPQSTDPLRTFKDTIYLTQVMQAQCVKTETEFYRRSRSEIVDQQGHTMGALYWQLNDIWQAPSWASLEYGGKWKMLHYFAQNFFAPLLPVGFENENTFYIYGVSDLHSDYSMTLSVRVHTWSSLEPVCSRVTERFVMKGGEAVCLYEEPVSELLRRCGNCTRESCVVSFYLSADHELLSPTNYHFLSSPKEAVGLCKAQITAIISQQGDIFVFDLETSAVAPFVWLDVGSIPGRFSDNGFLMTEKTRTILFYPWEPTSKNELEQSFHVTSLTDIY

SEQ ID NO: 14 - protein, 507 aa, Homo sapiens
MGAPRSLLLALAAGLAVARPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 15 - protein, 507 aa, Homo sapiens; identical to SEQ ID NO: 14

SEQ ID NO: 16 - protein, 522 aa, Homo sapiens
MAAVVAATRWWQLLLVLSAAGMGASGAPQPPNILLLLMDDMGWGDLGVYGEPSRETPNLDRMAAEGLLFPNFYSANPLCSPSRAALLTGRLPIRNGFYTTNAHARNAYTPQEIVGGIPDSEQLLPELLKKAGYVSKIVGKWHLGHRPQFHPLKHGFDEWFGSPNCHFGPYDNKARPNIPVYRDWEMVGRYYEEFPINLKTGEANLTQIYLQEALDFIKRQARHHPFFLYWAVDATHAPVYASKPFLGTSQRGRYGDAVREIDDSIGKILELLQDLHVADNTFVFFTSDNGAALISAPEQGGSNGPFLCGKQTTFEGGMREPALAWWPGHVTAGQVSHQLGSIMDLFTTSLALAGLTPPSDRAIDGLNLLPTLLQGRLMDRPIFYYRGDTLMAATLGQHKAHFWTWTNSWENFRQGIDFCPGQNVSGVTTHNLEDHTKLPLIFHLGRDPGERFPLSFASAEYQEALSRITSVVQQHQEALVPAQPQLNVCNWAVMNWAPPGCEKLGKCLTPPESIPKKCLWSH

SEQ ID NO: 17 - protein, 631 aa, Homo sapiens
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 18 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 19 - protein, 556 aa, Homo sapiens
MELCGLGLPRPPMLLALLLATLLAAMLALLTQVALVVQVAEAARAPSVSAKPGPALWPLPLLVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 20 - protein, 663 aa, Homo sapiens
MTGARASAAEQRRAGRSGQARAAERAAGMSGAGRALAALLLAASVLSAALLAPGGSSGRDAQAAPPRDLDKKRHAELKMDQALLLIHNELLWTNLTVYWKSECCYHCLFQVLVNVPQSPKAGKPSAAAASVSTQHGSILQLNDTLEEKEVCRLEYRFGEFGNYSLLVKNIHNGVSEIACDLAVNEDPVDSNLPVSIAFLIGLAVIIVISFLRLLLSLDDFNNWISKAISSRETDRLINSELGSPSRTDPLDGDVQPATWRLSALPPRLRSVDTFRGIALILMVFVNYGGGKYWYFKHASWNGLTVADLVFPWFVFIMGSSIFLSMTSILQRGCSKFRLLGKIAWRSFLLICIGIIIVNPNYCLGPLSWDKVRIPGVLQRLGVTYFVVAVLELLFAKPVPEHCASERSCLSLRDITSSWPQWLLILVLEGLWLGLTFLLPVPGCPTGYLGPGGIGDFGKYPNCTGGAAGYIDRLLLGDDHLYQHPSSAVLYHTEVAYDPEGILGTINSIVMAFLGVQAGKILLYYKARTKDILIRFTAWCCILGLISVALTKVSENEGFIPVNKNLWSLSYVTTLSSFAFFILLVLYPVVDVKGLWTGTPFFYPGMNSILVYVGHEVFENYFPFQWKLKDNQSHKEHLTQNIVATALWVLIAYILYRKKIFWKI

SEQ ID NO: 21 - protein, 743 aa, Homo sapiens
MEAVAVAAAVGVLLLAGAGGAAGDEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 22 - protein, 663 aa, Homo sapiens; identical to SEQ ID NO: 20

SEQ ID NO: 23 - protein, 552 aa, Homo sapiens
MRLLPLAPGRLRRGSPRHLPSCSPALLLLVLGGCLGVFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 24 - protein, 411 aa, Homo sapiens
MLLKTVLLLGHVAQVLMLDNGLLQTPPMGWLAWERFRCNINCDEDPKNCISEQLFMEMADRMAQDGWRDMGYTYLNIDDCWIGGRDASGRLMPDPKRFPHGIPFLADYVHSLGLKLGIYADMGNFTCMGYPGTTLDKVVQDAQTFAEWKVDMLKLDGCFSTPEERAQGYPKMAAALNATGRPIAFSCSWPAYEGGLPPRVNYSLLADICNLWRNYDDIQDSWWSVLSILNWFVEHQDILQPVAGPGHWNDPDMLLIGNFGLSLEQSRAQMALWTVLAAPLLMSTDLRTISAQNMDILQNPLMIKINQDPLGIQGRRIHKEKSLIEVYMRPLSNKASALVFFSCRTDMPYRYHSSLGQLNFTGSVIYEAQDVYSGDIISGLRDETNFTVIINPSGVVMWYLYPIKNLEMSQQ

SEQ ID NO: 25 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 26 - protein, 651 aa, Homo sapiens [sequence omitted]

SEQ ID NO: 27 - protein, 556 aa, Homo sapiens; identical to SEQ ID NO: 19